SEQUENCE LISTING (8 sequences)

SEQ ID NO: 1 - DNA, 25 bp, Artificial
  RT-PCR primer
aacaaaggccgcctgctgccttctc

SEQ ID NO: 2 - DNA, 25 bp, Artificial
  RT-PCR primer
gcagggcacaatgaccttgtcgaaa

SEQ ID NO: 3 - DNA, 25 bp, Artificial
  RT-PCR primer
aactgagcattagaaggtacatttg

SEQ ID NO: 4 - DNA, 25 bp, Artificial
  RT-PCR primer
tcaataggtccaatctgctctcaag

SEQ ID NO: 5 - DNA, 25 bp, Artificial
  RT-PCR primer
gctttatccctcttgtgatttacct

SEQ ID NO: 6 - DNA, 25 bp, Artificial
  RT-PCR primer
gtgaagagccttgtgccatccaact

SEQ ID NO: 7 - DNA, 25 bp, Artificial
  RT-PCR primer
acatggcctccaaggagtaagaccc

SEQ ID NO: 8 - DNA, 25 bp, Artificial
  RT-PCR primer
ggtactttattgatggtacatgaca

(19) United States
(12) Patent Application Publication
(10) Pub. No.: US 2009/0123925 A1
(43) Pub. Date: May 14, 2009

(54) CANCER THERAPY PROGNOSIS AND TARGET

(76) Inventors: Elaina S. R. Collie-Duguid, Aberdeen (GB); Keith Kerr, Aberdeen (GB); Russell Petty, Aberdeen (GB)

Correspondence Address: MYERS BIGEL SIBLEY & SAJOVEC, PO BOX 37428, RALEIGH, NC, 27627, US

(21) Appl. No.: 12/067,562

(22) PCT Filed: Sep. 25, 2006
(86) PCT No.: PCT/GB2006/003559

§ 371 Date: Oct. 14, 2008

(30) Foreign Application Priority Data
Sep. 23, 2005 (GB) 0519405.5

(51) Int. Cl. C12Q 1/68; C40B 40/08

(52) U.S. Cl. 435/6; 506/17

(57) ABSTRACT

The present invention relates to a gene and/or protein expression based method of predicting response to platinum based chemotherapy for lung cancer patients, as well as a method of predicting prognosis of survival based on protein expression and type of lung cancer. The invention also provides novel targets for screening candidate anti-cancer agents.

### FIELD OF THE INVENTION

The present invention relates to a gene and/or protein expression based method of predicting response to platinum based chemotherapy for lung cancer patients, as well as a method of predicting prognosis of survival based on protein expression and type of lung cancer. The invention also provides novel targets for screening candidate anti-cancer agents.

### INTRODUCTION

Non-small cell lung cancer (NSCLC) is a major global health care problem and is the most common cause of premature death from cancer¹. Improved understanding of the precise molecular mechanisms that underlie the biology of NSCLC and determine clinical outcomes will facilitate improved therapeutic approaches². The complexity and heterogeneity of these mechanisms has so far been a major obstacle to their full elucidation²⁻³. The diversity of individual clinical response to therapy is readily evident and the importance of unravelling the elaborate molecular networks behind this response is clearly apparent⁴⁻⁷. This would provide further insight into both the biological features of the disease that may be successfully exploited therapeutically and may allow prediction of response thereby facilitating a paradigm shift towards individualised therapy.

Global transcriptome profiling provides a means of addressing the complexity and heterogeneity of the molecular mechanisms governing tumorigenesis and underlying individual clinical behaviour. This technology has been used successfully in NSCLC and other malignancies to stratify each disease into new molecular sub-groups and in doing so provides novel insight into the mechanisms of disease aetiology and pathogenesis^{2,8-13}. Additionally these studies have provided gene expression profiles that have prognostic value for tumour recurrence following potentially curative treatment, with molecular signatures outperforming current clinicopathological methods for a variety of tumour types^{9,14-17}. Other, mostly preclinical studies have provided novel insight into the molecular mechanisms of response or resistance to cytotoxic agents¹⁸⁻²¹.

In certain embodiments, the present invention relates to the expression of Serpin B3 (SCCA1) and how its expression may be used as a predictive marker for response or resistance to platinum based chemotherapy, or as an aid to prognosis for NSCLC survival.

Serpin B3 (SCCA1) was initially described as a serum marker for squamous cell carcinoma of the cervix³⁸. Its role in cancer pathogenesis is not fully defined but over-expression of Serpin B3 has been demonstrated in squamous cell carcinomas of cervix, lung and head and neck, and hepatocellular carcinoma³⁸⁻⁴¹. In head and neck cancers, Serpin B3 expression in primary tumours is a poor prognostic factor⁴⁰. In addition to its role in negative regulation of cell death^{27,28}, in vitro studies have suggested a role in the inhibition of tumour cell invasion and metastasis, a process in which there is evidence of a role for several lysosomal cathepsin cysteine proteases, including the Serpin B3 substrate cathepsin L³⁵⁻³⁷. In NSCLC, there have been many contradictory reports of the diagnostic and prognostic value of SCCA (SerpinB3/SCCA1 and SCCA2)^{42-44.}

It is an object of the present invention to provide one or more methods suitable for use in predicting whether or not a NSCLC patient is likely to respond favourably or not to chemotherapy, particularly platinum based chemotherapy regimes.

Platinum based therapies are understood to include combination chemotherapy schedules using a platinum agent, e.g cisplatin, carboplatin, oxaliplatin or other new generation platinum agents, often administered in combination with other cytotoxics and are commonly used to treat lung cancers including NSCLC. However, not all patients respond to such treatment and the present invention in certain embodiments provides methods of predicting whether or not a cancer patient is likely to respond favourably to a platinum based chemotherapy (“responders”) i.e. show a reduction in tumour size, or not respond to platinum based chemotherapy (“non-responders”) i.e. not show a reduction in tumour size. As an example, reduction in tumour size may be determined by response evaluation criteria in solid tumors (RECIST) or by WHO response criteria (see Jpn J Clin Oncol 2003, 33(10) 533-537).

It is a further object of the invention to provide a method for providing a prognosis of survival of a NSCLC patient based on protein or gene expression and cancer typing.

It is a further object of the invention to provide a screen for identifying potential anti-cancer agents.

In a first aspect there is provided a method of predicting whether or not a cancer patient is suitable for chemotherapy, such as platinum based chemotherapy comprising the steps of:

a) providing a sample of non-small cell lung cancer (NSCLC) tumour tissue; and

b) detecting Serpin B3 expression in said tumour tissue, wherein if a proportion of tumour cells from the sample of tumour tissue are expressing Serpin B3, it is predicted that the patient is a poor candidate for response to chemotherapy and is not therefore suitable for chemotherapy.

The sample of NSCLC tumour tissue may have been obtained from a small biopsy taken from the tumour when in situ, i.e. before any surgical procedure to remove or resect the tumor, or may have been obtained from the tumour tissue once removed/resected from the patient, during surgery.

It is to be understood that a proportion of cells expressing Serpin B3 is typically understood to be greater than about 10% (i.e. between 10% and 100%) such as between 10-50%, preferably at least about 50%, in any sample of tumour tissue being tested. However, the proportion of cells expressing Serpin B3 may be difficult to quantify accurately and can be subjective and so a certain amount of variance is to be understood. Moreover, it is also understood that providing at least a clump or clumps of cells in the sample are seen as expressing Serpin B3, this may also be taken as a proportion of the cells from the sample of tumour tissue are expressing Serpin B3.

Detection may typically be carried out using labeling of Serpin B3 with an appropriate marker molecule. For example a labeled or unlabelled antibody or other binding agent specific for Serpin B3 may be used, to bind Serpin B3 and allow its detection. If an unlabelled antibody or other binding agent is used, it will be necessary to employ a labeling agent designed to bind to the antibody or binding agent, in order to allow detection. It may also be necessary to first permeabilise the cells in order to allow Serpin B3 to be detected and many techniques for achieving this are known to the skilled man and include microwaving the sample in 10 mM citrate (pH 6.0) for a period of time (e.g. 10, 20 or 30 mins).

Visualisation of the labeled Serpin B3 may be carried out manually using a microscope, with the user manually counting the labeled vs unlabelled cells in order to determine the proportion, which are expressing Serpin B3. Alternatively an automated system such as an optical or laser capture microscope (LCM) with associated software. An example is the Zeiss Axiovert 200M microscope with an Axiocan digital camera system for image analysis with Aphelion and Image Pro plus v software may be used to automatically count the cells and determine a percentage, which are expressing Serpin B3.

Values may be ascribed for proportions of cells in the sample expressing Serpin B3 and an indication of suitability or otherwise taken on a particular value. For example <10% cells expressing Serpin B3 may be given a value of 0; 10%-50%, a value of 1; and >50% a value of 2.

The inventors have observed that a high level of Serpin B3 expression is correlated with an unfavourable response to treatment of NSCLC with platinum based therapies, irrespective of tumour histological type. Thus, patients with a high level of Serpin B3 expression are unlikely to be good responders to platinum based therapies and the above method therefore may assist a physician to make an informed decision on how to treat his/her NSCLC patient.

Although the expression of Serpin B3 alone has been found to be a good predictor of response, it is envisaged that detecting other genes/proteins in combination with Serpin B3 expression may lead to an improved method.

Indeed, the inventors have also observed that protein expression levels of Serpin B3, cystatin C and Cathepsin B in pre-therapy tumour tissues are correlated with response to platinum based therapy and are independently predictive of response (independent of age, gender, smoking history, weight loss, performance status, stage, histological type and grade and chemotherapy regimen).

Performance status is determined according to WHO criteria and relates to the ability of a patient being able to conduct physical tasks.

Thus, in a second aspect there is provided a method of predicting whether or not a cancer patient is suitable for chemotherapy, such as platinum based chemotherapy, comprising the steps of:

a) providing a sample of non-small cell lung cancer (NSCLC) tumour tissue; and

b) detecting Serpin B3, cystatin C and cathepsin B expression in said tumour tissue, wherein if a proportion of tumour cells from the sample of tumour tissue are expressing Serpin B3, and cystatin C relative to cathepsin B, it is predicted that the patient is a poor candidate for response to chemotherapy and is not therefore suitable for chemotherapy.

Detection may be carried out similarly as described above and likewise values given to Serpin B3, cystatin C and cathepsin B expression, in order to provide a score value for Serpin B3 and cystatin C/cathepsin B expression.

The inventors have observed that a combined level of Serpin B3 and cystatin C over cathepsin B can be used as a predictor of response. A combined immunohistochemical score (Serpin B3+cystatin C/cathepsin B) can be used to predict response (Accuracy 72%, positive predictive value for non-response 90%, sensitivity response 94%, specificity 64% and sensitivity non-response 53%, specificity 91%). It is proposed that high sensitivity for response combined with high specificity for non-response would allow the majority of responding patients (94%) to be prospectively identified and treated with platinum based therapy whilst allowing 53% of non-responding patients to be prospectively identified, potentially allowing first-line treatment with a novel regimen, precluding toxic treatment of these patients with an ineffective drug—this would be using the protein expression levels of Serpin B3, cystatin C and cathepsin B in the tumour tissues (Serpin B3+cystatin C/cathepsin B; threshold cut-off=2). See the section on immunohistochemistry, hereinafter, for a discussion on scoring values. For the purpose of the present invention a value of greater that 2 e.g. 2.5, 3, 3.5, 4, etc. is understood to mean that the patient is likely to be a non-responder and may not therefore be suitable for platinum based therapy.

Other genes the expression of which in addition to Serpin B3, may be of importance in prediction of response, may include the substrates of Serpin B3. These substrates include cathepsins L, K and S and papain.

In addition to the above aspects, the present inventors have identified further genes, including a 17 gene set, which includes Serpin B3, that is even more strongly correlated with response to platinum based therapy in NSCLC patients and forms the basis of a more improved method of predicting response to chemotherapy.

Thus, in a third aspect there is provided a method of predicting whether or not a cancer patient is suitable for platinum based chemotherapy, comprising the steps of:

a) providing a sample of non-small cell lung cancer (NSCLC) tumour tissue;

b) detecting a level of expression of Serpin B3 and one or more of the following 16 genes:

Hypothetical Protein FLJ23049 (FLJ23049), Hydroxysteroid (17β) dehydrogenase 2 (HSD17B2), Cell Division Cycle homolog 20 (CDC20), Fibronectin type 3 domain containing 3A (FNDC3A), Hypothetical Protein FLJ11767 (FLJ11767—now identified as EF-hand calcium binding domain 1 [EFCAB1]), Semaphorin 3D (SEMA3D), Cystatin SN (CST1), Sperm Associated Antigen 6 (SPAG6), Potassium inwardly rectifying channel subfamily J member 16 (KCNJ16), Histone 1H2bg (H1ST1H₂BG), testicular Soluble Adenylyl Cyclase (SAC), Gelsolin (GSN), Cellular Retinoic Binding Protein 1 (CRABP1), Carbonic Anhydrase isoform 12 (CA12), Zinc Finger protein 444 (ZNF444), and v-myb myeloblastosis viral oncogene homolog (MYB); and

c) predicting whether or not the patient is likely to be a responder or non-responder to a chemotherapy based on a profile of expression of said genes and therefore suitable or otherwise for chemotherapy treatment.

Generally speaking, the more genes/proteins which are included may lead to an improved predictor of response and especially using genes/proteins which display a predictive strength of greater than 4, see hereinafter. Thus, the method of the third aspect may preferably include at least 5, 10, 11, 12, 13, 14, 15 or 16 of said genes and may optionally include further expressed genes which display a significant difference in expression in NSCLC tissues between the responding and non-responding patients described in the methods below, typically a 2, 3 or 4-fold difference in expression.

The inventors found that the profile of expression levels of the 17 genes identified above in NSCLC tumour tissues obtained at the surgical resection was strongly correlated with response. These tumour tissues included pre-therapy (i.e. from patients not previously subjected to chemotherapy prior to resection) and post-therapy (i.e. patients who had received chemotherapy prior to surgical resection of the tumour) tissues. Pre-therapy tumour tissues were obtained between 3 and 37 months prior to therapy suggesting that the levels of these genes at time of surgery remain predictive of response over a long period. Prediction of whether or not a patient is likely to be a responder or non-responder, based on a profile of expression is carried out using appropriate computer software. Image acquisition with initial normalisation and/or filtering of data may be performed using specialised software provided by the manufacturer, for example MAS, and microDB or GCOS with DMT software respectively, available from Affymetrix, Santa Clara, Calif. Further threshold and probabilistic filtering, supervised analysis using gene ontologies, hierarchical cluster analysis and leave-one-out cross-validation using Fishers exact t test hypergeometric probability and KNV may be performed using commercial software packages designed for analysis of gene expression data, for example GeneSpring (now Agilent, Silicon Genetics, Redwood City, Calif.).

In addition to SerpinB3, a further 10 cell death genes were identified as being highly correlated with response to platinum based chemotherapy in NSCLC patients.

Thus in a fourth aspect there is provided a method of predicting whether or not a cancer patient is suitable for platinum based chemotherapy, comprising the steps of:

a) providing a sample of non-small cell lung cancer (NSCLC) tumour tissue;

b) detecting a level of expression of Serpin B3 and one or more of the following 10 genes:

Collagen Type IV alpha 3 chain (COL4A3), Angiotensin II receptor type 2 (AGRT2), Cystatin C (CST3), Survivin (BIRC5), Dual specificity phosphatase 6 (DUSP6), TNF superfamily receptor member 21 (TNFRS21), STAT1 (STAT1), Epithelial Membrane Protein 3 (EMP3), Tissue Inhibitor of Metalloprotease-3 (TIMP3) and Nucleophosmin (NPM1); and

c) predicting whether or not the patient is likely to be a responder or non-responder to a chemotherapy based on a profile of expression of said genes and therefore suitable or otherwise for platinum based chemotherapy treatment.

Generally speaking at least 4, 5, 6, 7, 8, 9 or 10 of said genes are employed in the method and optionally other cell death genes identified as showing a significant difference in expression between normal and NSCLC tissue.

To further improve the above methods Serpin B3 and all 26 genes (i.e. 16+10 identified above) may be used in order to predict response to platinum based chemotherapy.

Thus, in a fifth aspect there is provided a method of predicting whether or not a cancer patient is suitable for platinum based chemotherapy, comprising the steps of:

a) providing a sample of non-small cell lung cancer (NSCLC) tumour tissue;

b) detecting a level of expression of the following genes: Serpin B3 (SERPINB3), Hypothetical Protein FLJ23049 (FLJ23049), Hydroxysteroid (17β) dehydrogenase 2 (HSD17B2), Cell Division Cycle homolog 20 (CDC20), Fibronectin type 3 domain containing 3A (FNDC3A), Hypothetical Protein FLJ11767 (FLJ11767, now identified as EF-hand calcium binding domain 1 [EFCAB1]), Semaphorin 3D (SEMA3D), Cystatin SN (CST1), Sperm Associated Antigen 6 (SPAG6), Potassium inwardly rectifying channel subfamily J member 16 (KCNJ16), Histone 1H2bg (H1 ST1H2BG), Testicular Soluble Adenylyl Cyclase (SAC), Gelsolin (GSN), Cellular Retinoic Binding Protein 1 (CRABP1), Carbonic Anhydrase isoform 12 (CA12), Zinc Finger protein 444 (ZNF444), and v-myb myeloblastosis viral oncogene homolog (MYB), Collagen Type IV alpha 3 chain (COL4A3), Angiotensin II receptor type 2 (AGRT2), Cystatin C (CST3), Survivin (BIRC5), Dual specificity phosphatase 6 (DUSP6), TNF superfamily receptor member 21 (TNFRS21), STAT1 (STAT1), Epithelial Membrane Protein 3 (EMP3), Tissue Inhibitor of Metalloprotease-3 (TIMP3); and Nucleophosmin (NPM1); and

c) predicting whether or not the patient is likely to be a responder or non-responder to a platinum based chemotherapy based on a profile of expression of said genes and therefore suitable or otherwise for platinum based chemotherapy treatment.

According to the above, expression can be assayed by analysing and/or quantifying the nucleic acid (including mRNA, product of gene transcription) or protein (including short peptide and other protein translation products) products of gene expression. Methods for measuring gene expression are known in the art, and examples are discussed herein. However, one ordinary skill in the art will understand that methods of the invention relate to all assays of gene expression in normal or diseased lung samples.

The present invention also provides arrays of gene expression detection agents for use in the methods of the present invention.

Thus, in a further aspect, there is provided a DNA array for use in a method according to the third to fifth aspects, the array comprising or consisting essentially of Serpin B3 and one or more of the following genes or sequence specific fragments thereof: Hypothetical Protein FLJ23049 (FLJ23049), Hydroxysteroid (17β) dehydrogenase 2 (HSD17B2), Cell Division Cycle homolog 20 (CDC20), Fibronectin type 3 domain containing 3A (FNDC3A), Hypothetical Protein FLJ11767 (FLJ11767—now identified as EF-hand calcium binding domain 1 [EFACB1]), Semaphorin 3D (SEMA3D), Cystatin SN (CST1), Sperm Associated Antigen 6 (SPAG6), Potassium inwardly rectifying channel subfamily J member 16 (KCNJ16), Histone 1H2bg (H1ST1H2BG), Testicular Soluble Adenylyl Cyclase (SAC), Gelsolin (GSN), Cellular Retinoic Binding Protein 1 (CRABP1), Carbonic Anhydrase isoform 12 (CA12), Zinc Finger protein 444 (ZNF444), and v-myb myeloblastosis viral oncogene homolog (MYB), and/or Collagen Type IV alpha 3 chain (COL4A3), Angiotensin II receptor type 2 (AGRT2), Cystatin C (CST3), Survivin (BIRC5), Dual specificity phosphatase 6 (DUSP6), TNF superfamily receptor member 21 (TNFRS21), STAT1 (STAT1), Epithelial Membrane Protein 3 (EMP3), Tissue Inhibitor of Metalloprotease-3 (TIMP3); and Nucleophosmin (NPM1), the array being immobilised on a support.

Preferably the array is a DNA microarray. Advantageously the array or microarray is prepared on any suitable, preferably non-porous substrate. Typically the suitable substrate may include glass or a plastic material. Information regarding suitable substrates and the protocols used to generate arrays or DNA microarrays may be obtained from the National Human Genome Research Institute, Bethesda USA.

Generally the surface of the suitable microarray substrate is treated in someway so that nucleic acid specific to said genes, that is nucleic acid corresponding to said gene or specific fragment thereof may be coupled to it. For example the surface of the suitable substrate may be made hydrophobic so as to prevent spread of individual nucleic acid samples applied to the microarray substrate and positively charged so as to facilitate the coupling of the nucleic acid to the microarray substrates. Such a hydrophobic/positively charged surface may be obtained by use of a substance such as poly-L-lysine.

After such preparation of the microarray substrate, the nucleic acid fragments may be spotted on to the surface as an array. Preferably automated printing procedures known in the art may be utilised to apply the nucleic acid fragments as an array. Alternatively, custom-made DNA microarrays synthesised by in situ synthesis of oligonucleotide or other nucleic acid probes on the surface by, for example, photolithographic technology performed using a proprietary technology by Affymetrix, Santa Clara, Calif.

Preferred gene expression detection agents hybridise specifically to the genes identified herein whose expression is correlated with response to platinum based chemotherapy. Such agents may be RNA, DNA or PNA molecules. Preferred agents are fragments of the above identified genes, e.g. oligonucleotides specific therefore. Alternative agents may bind specifically to the protein expression products of the marker genes disclosed herein. Preferred agents include antibodies and aptamers.

Agents, such as oligonucleotides, are preferably attached to a solid support in the form of an array. Oligonucleotide arrays in the form of DNA microarrays and useful hybridisation assays are known in the art and disclosed for example in U.S. Pat. Nos. 5,631,734; 5,874,219; 5,861,242; 5,585,659; 5,856,174; 5,843,655; 5,837,832; 5,834,758; 5,770,722; 5,770,456; 5,733,729; 5,556,752; 6,045,996 and 6,261,776. In a preferred embodiment, an array includes oligonucleotides for measuring the expression level of the genes identified herein. However, it is also possible to use cDNAs or PCR products for probes.

The present invention further provides a database of said identified genes and information about the said genes, including the expression levels that are characteristic of NSCLC platinum based chemotherapy responders and non-responders. According to the invention, said gene information is preferably stored in a memory in a computer system. Alternatively, the information is stored in a removable data medium such as a magnetic disk, a CDROM, a tape, or an optical disk. In a further embodiment, the input/output of the computer system can be attached to a network and the information about the identified genes can be transmitted across the network.

Preferred information includes the identity of the genes identified herein, the expression of which correlates with an expected response to a platinum based chemotherapy regime. In addition, threshold expression levels of said genes may be stored in a memory or on a removable data medium. According to the invention, a threshold expression level is a level of expression of the marker gene that is indicative of response or non-response to platinum based chemotherapy.

In a highly preferred embodiment, a computer system or removable data medium includes the identity and expression information. In addition, information about expression levels of said genes for normal lung tissue may be included.

The present inventors have also observed that Serpin B3 expression in tumours can be used as a prognostic marker of patient survival time, but that this is dependent on histological type and nodal stage of the tumour as described hereinafter.

Thus, in a sixth aspect there is provided a method of predicting a prognosis of a patient's survival following surgical resection of a non-small cell lung cancer tumour (NSCLC) tumour, comprising the steps of:

a) providing a sample of said surgically resected tumour;

b) detecting Serpin B3 expression in said tumour tissue sample; and

c) determining whether or not said tumour was of the squamous cell carcinoma (SCC) or adenocarcinoma (AC) type and detecting lymph node status if said tumour was of the SCC type, wherein if a significant proportion of tumour cells in said sample are expressing Serpin B3 and the type of tumour is a SCC tumour of the N0 or N1 lymph node status, a good prognosis for the patient is predicted and wherein if a significant proportion of the tumour cells in said sample are expressing Serpin B3 and the type of tumour is an AC tumour or SCC tumour of the N2 or N3 lymph node status, a poor prognosis for the patient is predicted.

Scoring may be carried out in a manner similar to that described above. For example, score 0 or 1 (no staining or a few individual cells staining (e.g. about <10%)) is a low score and score of 2 (e.g. about 10-50%) or 3 (about >50%) is a high score.

Prognosis relates to cumulative survival and is generally understood to provide a likelihood of survival at 5 years. As an example, the inventors have observed that a NSCLC patient with AC and a high level of Serpin B3 expression is 2.09 times more likely to be dead at 5 years than a patient with the same tumour type, but low Serpin B3—10% are alive vs 37%. In summary, in the scenario outlined above, high/significant expression of Serpin B3 gives a poor prognosis for survival at 5 years. However, the relationship between SerpinB3 and survival is dependent on histology and lymph node status as outlined below.

Without wishing to be bound by theory, the inventors believe that where SerpinB3 mediated inhibition of invasion is involved, the prognosis difference comes into play ˜3 years (SCC N0/N1), but where it may be a putative cell death role for SerpinB3, the prognosis difference is virtually at outset; 1-2 yrs in NSCLC patients who have adenocarcinomas, irrespective of lymph node status —N0, N1, N2 or N3, see below), or SCC with N2 or N3 status. High/significant Serpin B3 expression gives a good prognosis for survival for NSCLC patients with squamous cell carcinomas with N0 or N1 disease, but a poor prognosis for patients with either AC or with squamous cell carcinoma with N2 or N3 disease.

Although the “N” stages of NSCLC are well understood, for the avoidance of doubt, these are defined as follows: N0: No spread to lymph nodes; N1: Spread to lymph nodes within the lung and/or located around the area where the bronchus enters the lung (hilar lymph nodes). Metastases affect lymph nodes only on the same side as the cancerous lung; N2: Spread to lymph nodes around the point where the windpipe branches into the left and right bronchi or in the space behind the chest bone and in front of the heart (mediastinum). Affected lymph nodes are on the same side of the cancerous lung; and N3: Spread to lymph nodes near the collarbone on either side or to hilar or mediastinal lymph nodes on the side opposite the cancerous lung.

Finally, the present invention provides methods for identifying, evaluating, and/or monitoring drug candidates for the treatment of NSCLC. According to the invention, a candidate drug may be assayed for its ability to modulate the expression of Serpin B3 and/or any of the other genes/proteins identified herein in a NSCLC tumour. In one embodiment, a specific drug may reduce the expression of Serpin B3 and/or any of the other genes/proteins identified herein for a specific type and/or subclass of NSCLC described herein. Alternatively, a preferred drug may have a general effect on lung cancer and decrease the expression of Serpin B3 and/or any of the other genes/proteins identified herein. In order to avoid repetition, mention hereinafter will only be made to Serpin B3 as a target drug candidate, but this should not be construed as limiting.

In one embodiment, a candidate drug may be added to cells or sample tissue prior to analysis. Preferred cells are cell lines grown from different types of NSCLC (e.g. different classes or subclasses and/or stages of NSCLC). Alternatively, cells isolated directly from tumour tissue can be assayed.

In another embodiment, the invention provides screens for a candidate drug which modulates lung cancer, modulates lung cancer Serpin B3 gene expression and/or protein expression, modulates lung cancer Serpin B3 gene or protein activity, binds to Serpin B3 in a lung cancer tissue, or interferes with the binding of Serpin B3 in lung cancer tissue to its substrates.

The term “candidate drug” or equivalent as used herein describes any molecule, e.g. an antibody or antibody fragment, protein, oligopeptide, fatty acid, steroid, small organic molecule, polysaccharide, polynucleotide, RNAi molecule, antisense molecule, ligand, bioactive partner and structural analogues or combinations or conjugates thereof, to be tested for candidate drugs that are capable of directly or indirectly altering the lung cancer resistant phenotype, or the expression of Serpin B3. Such candidate drugs may also be administered in combination with an agent designed to facilitate entry into a cell and many such agents are known in the art.

The amount of gene expression can be monitored at either the gene level or the protein level, i.e. the amount of gene expression may be monitored using nucleic acid probes and methods known in the art to quantify gene or protein expression levels and as described herein. Alternatively, the Serpin B3 protein can be monitored, for example through the use of antibodies to Serpin B3 in standard immunoassays.

The present invention will now be further described by way of example and with reference to the tables and figures, which show:

Table 1 shows clinicopathological details of patients comprising training set of 8 consecutive NSCLC patients treated with neoadjuvant platinum-based chemotherapy prior to surgical resection of their tumour, which was profiled in gene expression analysis. Chemotherapy schedules are described in methods;

Table 2 shows genes whose expression is highly correlated with clinical sensitivity or resistance to platinum combination chemotherapy in NSCLC patients. These 17 genes constitute the “predictive gene set”. Discussed in detail in the text. ¹Prediction strengths were evaluated for all genes and provide a measure of the degree of association of the expression of the gene with clinical response. To calculate predictive strength, all genes were evaluated independently by their ability to discriminate each response group (non-response versus response) with Fishers exact t test hypergeometric probability, using expression data from that gene alone. The predictive strength is the negative natural log of the p-value. ²Fold change represents the mean normalized gene expression in non-responding patients/mean normalized gene expression in responding patients. ³The training set is a series of 8 consecutive patients with resectable NSCLC, used in the generation of the molecular classifier. ⁴The test set is an independent series of NSCLC primary tumour tissues not utilised in the generation of the molecular signature and includes early and advanced stage patients. ⁵This set contains only data from pre-chemotherapy tumour tissues within the independent test set. ⁶This is the combined training and test set data. *Indicates the probe set for which expression data is shown where more than one probe set from the same gene was obtained in the analysis;

Table 3 shows clinicopathological details of patients comprising the independent test set. LT9 and 10 are patients treated with neoadjuvant chemotherapy (post-chemotherapy tissues used) and patients LT11-16 are patients treated with chemotherapy on metastatic relapse after prior surgical resection (tissues used are pre-chemotherapy resection specimens 3-37 months prior to relapse and chemotherapy administration). Chemotherapy schedules are: MVP is Mitomycin C (8 mg/m²-cycles 1 and 2 only), Vinblastine (6 mg/m²) and Cisplatin (50 mg/m²) administered every 21 days; NP is Vinorelbine (30 mg/m²) and Cisplatin (80 mg/m²) administered every 21 days; Gemcitabine (1250 mg/m²) and Cisplatin (60 mg/m²) administered every 21 days; Carboplatin (AUC6) and Paclitaxel (225 mg/m²) administered every 21 days; Cisplatin (60 mg/m²) and Paclitaxel (225 mg/m²) administered every 21 days; Carboplatin (AUC 6) and docetaxel (75 mg/m²) administered every 21 days.

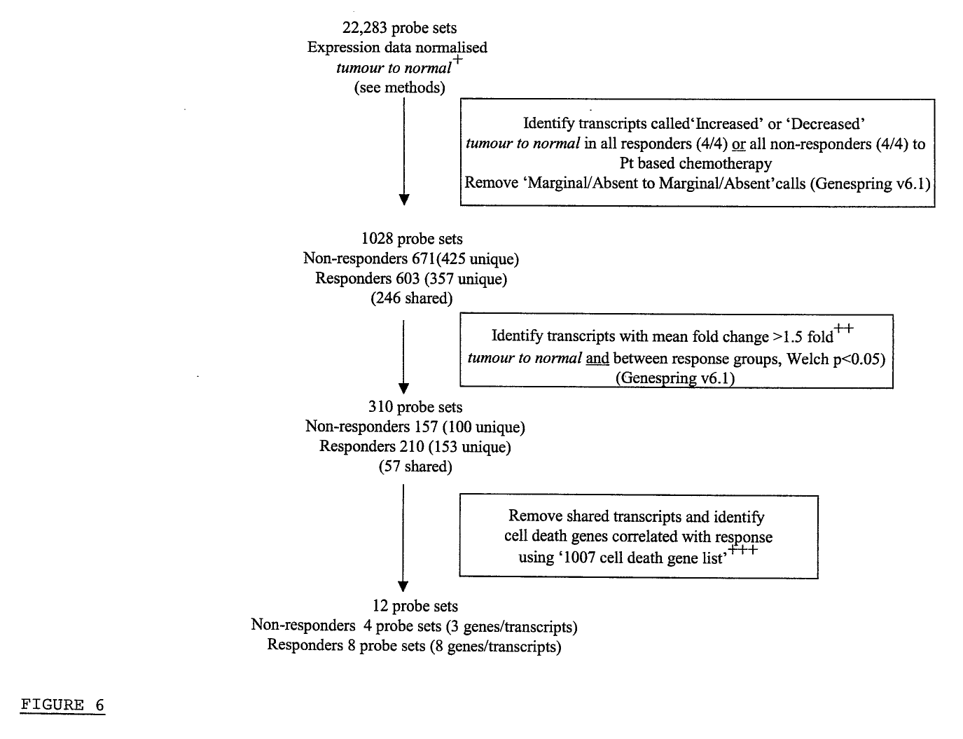

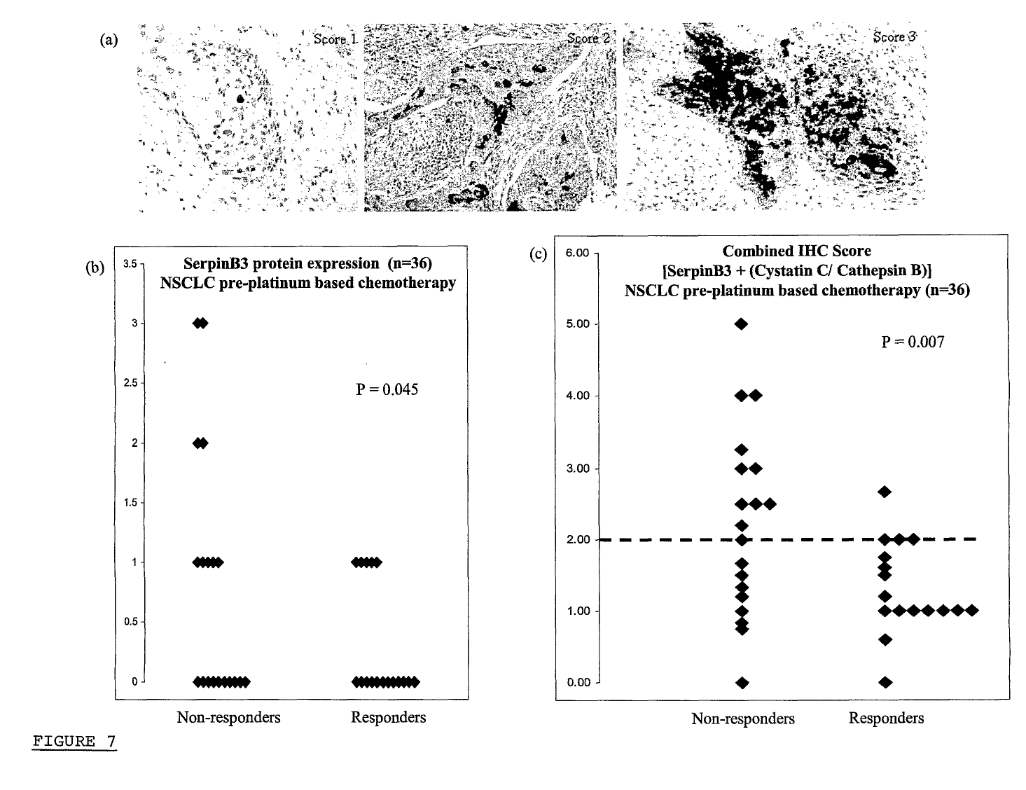

Table 4 shows cell death genes, whose expression is consistently, significantly and specifically correlated with clinical non-response (a) or response (b) to platinum-based combination chemotherapy in NSCLC patients. Based upon analysis of 1007 cell death genes represented on the HGU133A microarray, as discussed in detail in FIG. 6 and the text. See also supplementary Table 1. ¹This is the mean of the normalised expression in each tumour relative to the normalised expression in matched uninvolved adjacent “normal” lung. ²This is the fold change between mean normalised expression in each response group [NR:R (a) or R:NR (b)];

Table 5 shows clinicopathological details of patients treated with Pt based combination chemotherapy used for immunohistochemical analysis of lysosomal proteins as discussed in text. A) Pre-treatment (n=36) or B) post-treatment biopsies (n=13);

Table 6 Summary of immunohistochemical analysis of SerpinB3, Cystatin C and Cathepsin B protein expression. Correlation between expression of lysosomal cysteine protease inhibitor, Serpin B3, identified from gene expression profiling, and clinical response in an independent set of NSCLC patients treated with platinum based combination chemotherapy (36 pre-treatment biopsies and 13 post-treatment biopsies, details in table 5a and 5b, respectively). A combined IHC score, representing activity of the 2 lysosomal proteases identified, SerpinB3 and Cystatin C relative to its main identified physiological target cysteine protease, Cathepsin B, was highly correlated with response. Protein expression of either Cystatin C or Cathepsin B alone was not significantly correlated with response (p>0.05; not shown);

Table 7 shows clinicopathological details of NSCLC patients, who had not received any chemotherapy. This chemotherapy-naïve population was used for investigation of the prognostic value of Serpin B3 protein expression using immunohistochemical analysis of a lung squamous cell carcinoma tissue microarray (n=176) and stage, grade and age matched adenocarcinomas (n=75); and

Table 8 shows Serpin B3 protein expression (Negative (IHC score 0) vs. positive (IHC 1-3)), measured by immunohistochemistry in chemotherapy-naive lung squamous cell carcinomas and stage, age and grade matched chemotherapy-naïve adenocarcinomas. See text for further details and discussion.

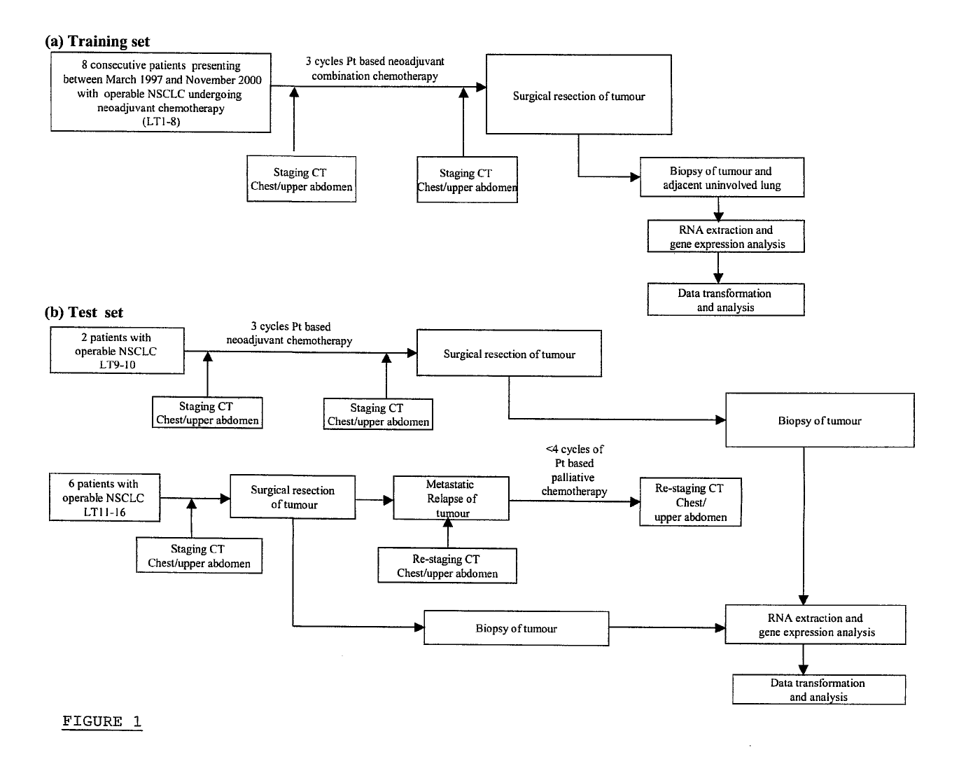

FIG. 1 shows a schematic representation of sample accrual for RNA extraction and subsequent oligonucleotide microarray (22,283 probe sets) analysis (more details in text). A) Training set used for generation of the molecular classifier B) Independent test set not used in identification of response markers.

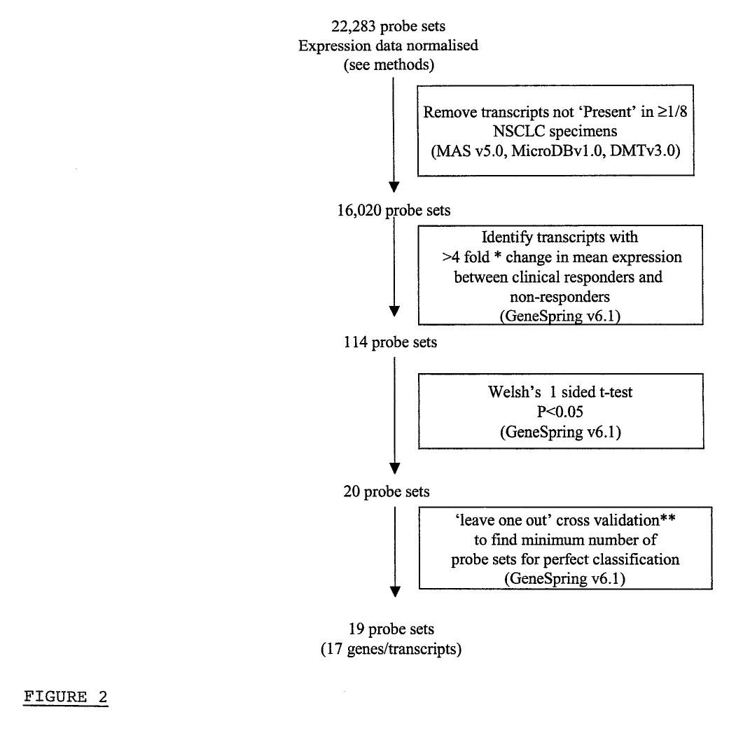

FIG. 2 shows a schematic illustrating analysis of gene expression data to obtain a “predictive gene set” whose expression is highly correlated with clinical response. *Reproducibility experiments (not shown) of adjacent biopsies of normal lung tissue from the same specimen resulted in 0.016% false positives using a threshold cut-off of 4-fold; **“leave one out cross-validation” based on Fishers exact t test hypergeometric probability and k nearest neighbours (k=3), correctly classified all tumours in the training set according to response.

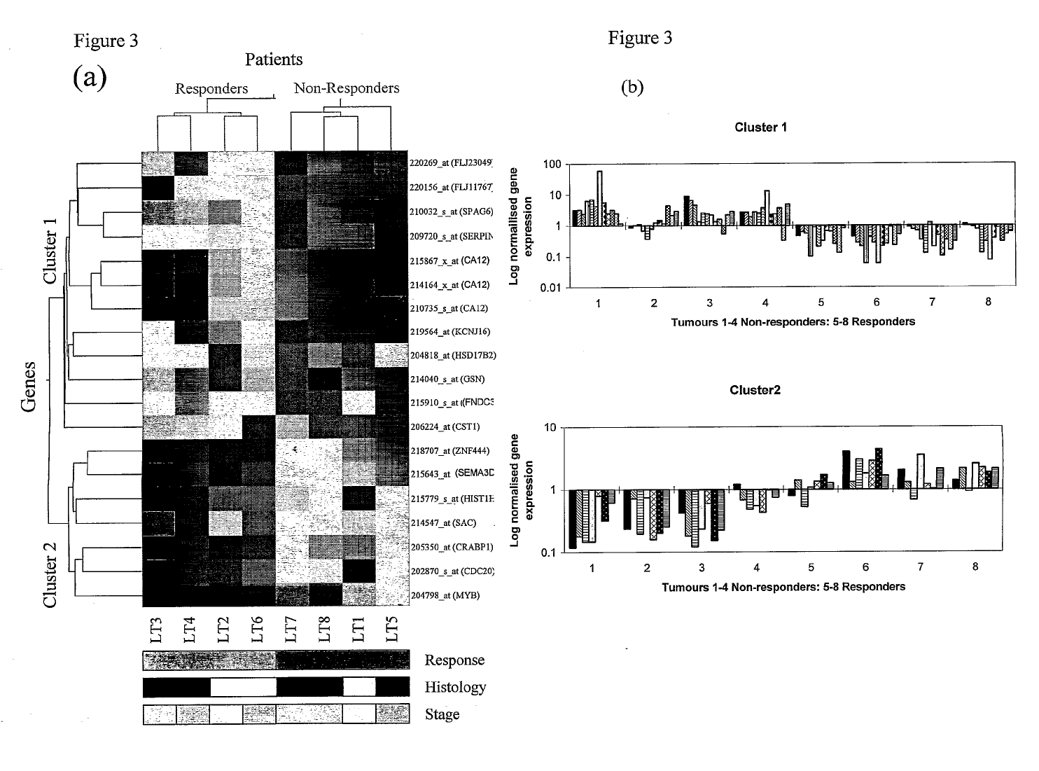

FIG. 3

(a) Dendrogram and colour plot illustrating clustering of patients in training set (n=8). Hierarchical cluster analysis using standard correlation of log transformed normalised gene expression data from the predictive gene set (n=17). Columns represent tumours from individual patients and rows represent genes with up-regulated (black) or down-regulated (grey) expression. The genes are grouped into 2 dominant gene clusters according to expression patterns. Probe set ID and gene symbols are shown to the right. Tumours are separated into 2 primary clusters representing non-responding (grey) or responding (black) tumours. Clustering of tumours based on gene expression data was not correlated with the histological type (AC (black) or SCC (white)) or stage (IB (grey); IIB (black); IIIA (spotted)).
(b) Log transformed normalised gene expression levels of genes in the 2 dominant gene clusters in predictive gene set. Cluster 1 contains 10 genes (12 probe sets) primarily over-expressed in resistant tumours, whereas cluster 2 contains 7 genes primarily over-expressed in responding tumours.

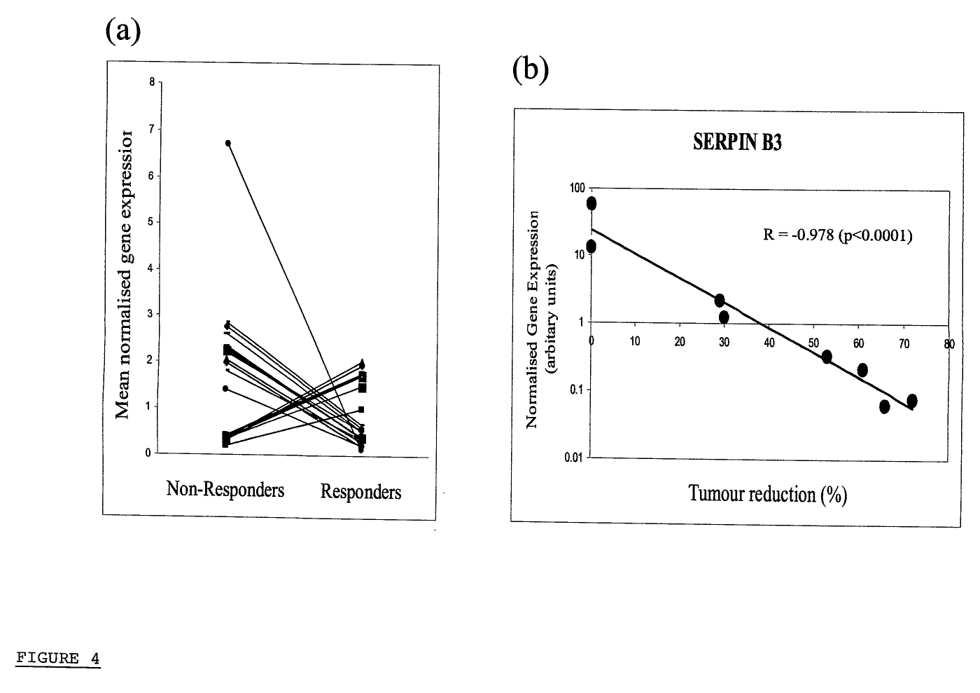

FIG. 4 shows A) Expression of predictive gene set (table 2) in different response groups. Mean normalised expression in tumours from non-responding patients vs. responding patients, demonstrates Serpin B3 is an outlier in this series.

B) Serpin B3 mRNA expression shows a highly significant correlation with the degree of tumour response on CT scan. R is Spearman's Rank correlation coefficient, % response defined as (Product of maximal perpendicular diameters after chemotherapy/Product of maximal perpendicular diameters before chemotherapy* 100). No correlation seen between Serpin B3 expression and tumour cellularity (not shown).

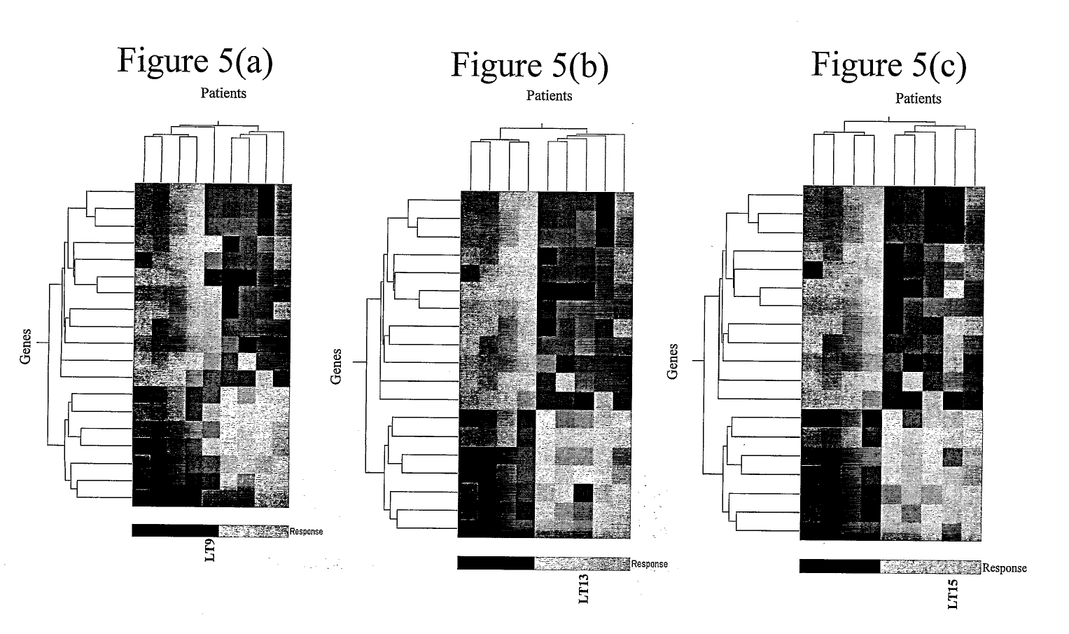

FIG. 5

Dendrogram and colour plot of hierarchical clustering, showing correct grouping according to response for independent test samples. 8/8 of the test samples cluster correctly for response, using standard correlation of log transformed normalised gene expression data. An early disease patient (IB (a)) and 2 advanced stage patients (IIIA (b) and IIB (c)) are shown. Columns represent tumours from individual patients and rows represent genes with up-regulated (black) or down-regulated (grey) expression. The genes group into 2 dominant clusters according to expression patterns. Tumours separate into 2 primary clusters representing non-responding (grey) or responding (black) tumours.

FIG. 6 shows a schematic illustrating supervised analysis of cell death pathways to identify genes whose expression is consistently, significantly and specifically altered according to clinical response. ⁺Normalised gene expression data for individual tumours was expressed relative to the normalised gene expression in each tumour's matched uninvolved normal lung control. ⁺⁺Consistent 1.5 fold threshold cut-off for gene expression measured on microarrays was validated at the protein level in previous work²⁴. ⁺⁺⁺Using gene ontologies (RefSeq, UniGene and LocusLink, GeneSpring v6.1 and literature searches, PubMed, ISI), we identified 1007 genes (see supplementary Table 1) involved in the execution and control of cell death pathways (both apoptotic and non-apoptotic, caspase dependent and independent).

FIG. 7 shows (a) Photomicrographs showing Serpin B3 protein expression in NSCL tumour cells, using immunohistochemistry. Cytoplasmic staining is seen. Representative examples of scoring categories 1-3 are shown (200×).

(b) Serpin B3 protein levels measured by IHC are highly correlated with clinical response in 36 tumours (pre-chemotherapy tissues, p=0.045. Figure illustrates, high scores >1 are invariably found in non-responding tumours.
(c) Combined IHC score of the 3 proteins evaluated, designed to reflect protease inhibition (SerpinB3+Cystatin C/Cathepsin B) is highly correlated with clinical response in 36 tumours (pre-chemotherapy tissues, p=0.007), where high scores (>2) are almost invariably associated with resistance (Positive predictive value for non-response is 90%).

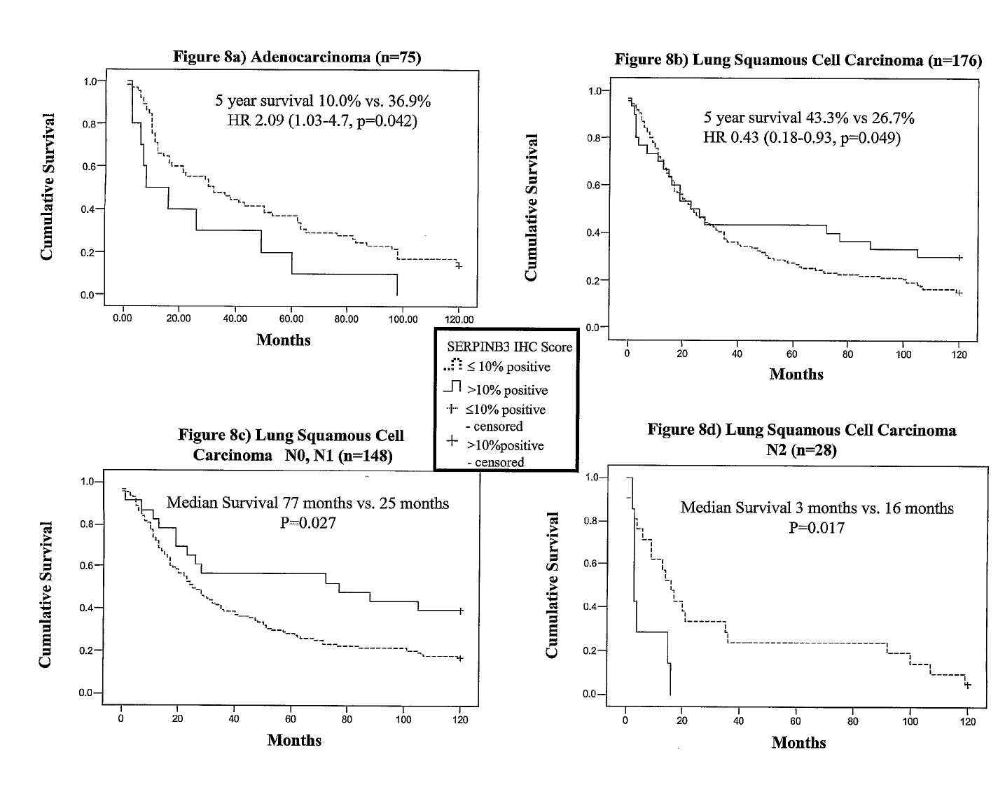

FIG. 8 shows Kaplan Meier survival analysis reveals contrasting prognostic impact of SerpinB3 protein expression measured by immunohistochemistry in pulmonary adenocarcinomas (a) and squamous cell carcinomas (b) and according to nodal status in squamous cell carcinomas (c and d), but not in adenocarcinomas (not shown). Discussed in detail in text.

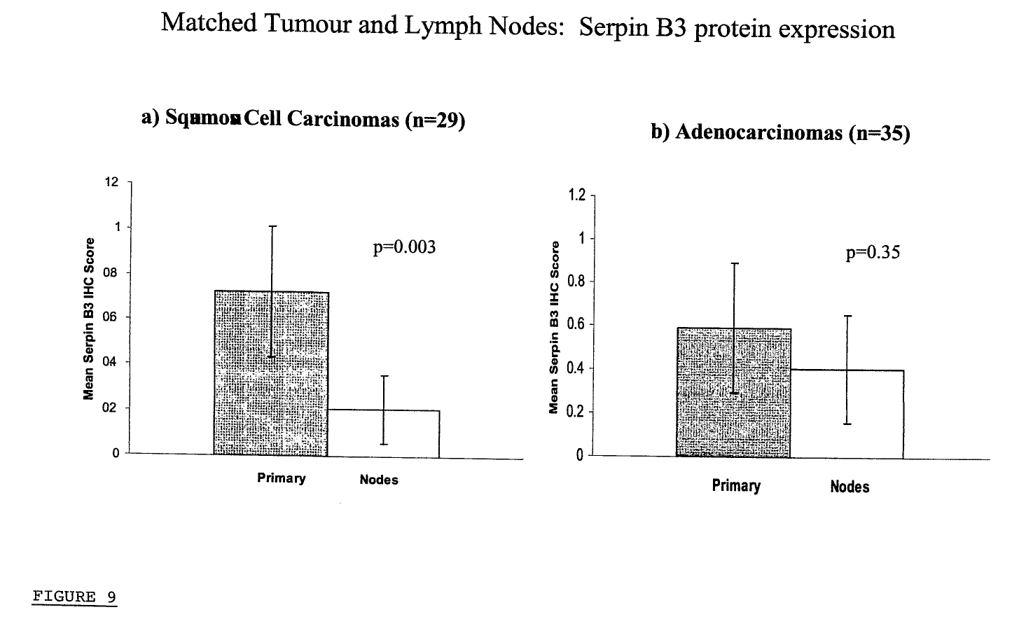

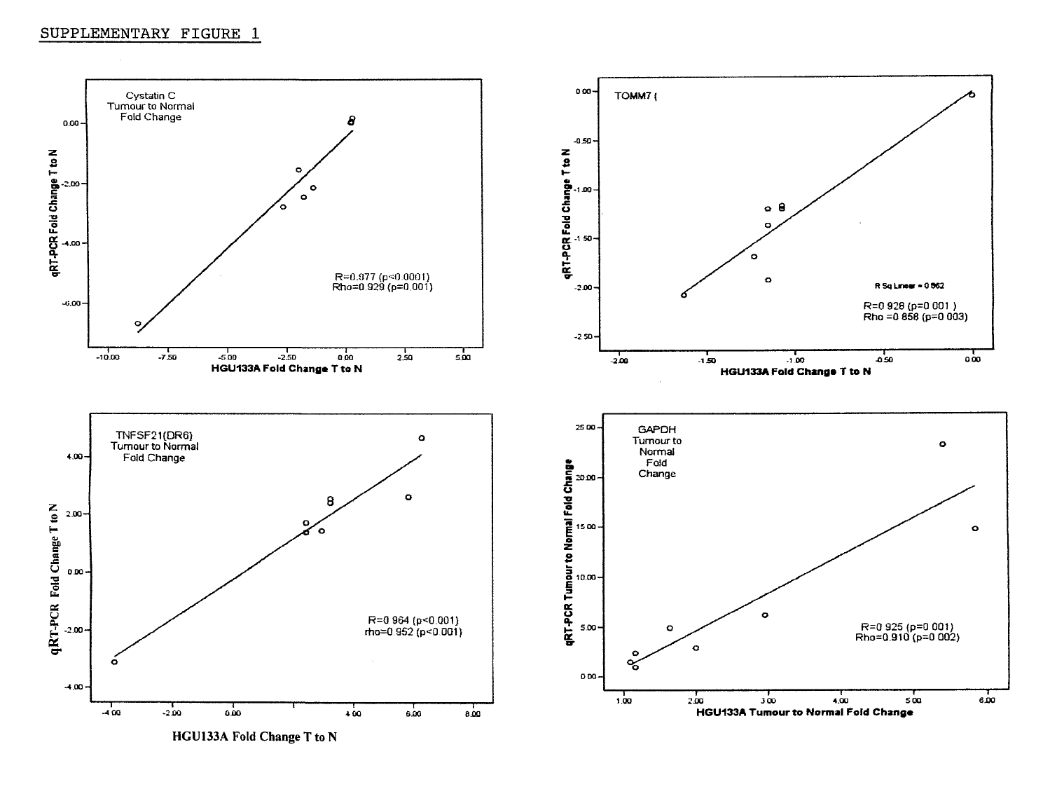

FIG. 9 shows contrasting patterns of Serpin B3 protein expression assessed by immunohistochemistry in matched primary tumour and regional lymph node metastasis in lung squamous cell carcinomas and adenocarcinomas, consistent with a role for Serpin B3 in inhibition of invasion and metastases in squamous cell carcinomas but not adenocarcinomas.

Supplementary FIG. 1 shows results of real time RT-PCR reactions.

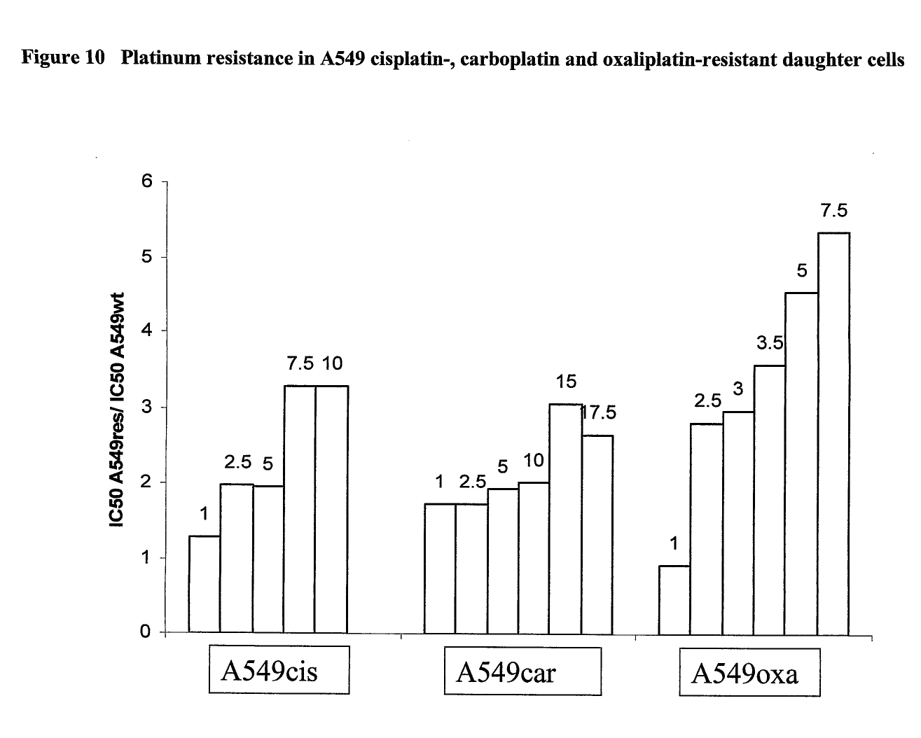

FIG. 10 shows illustration of the level of resistance of cisplatin-, carboplatin- and oxaliplatin-resistant A549 and oxaliplatin-resistant H630 cells compared to the sensitive parental line. Fold change is IC50 of cisplatin (A549cis), carboplatin (A549car) or oxaliplatin (A549ox and H630ox) in the respective resistant line vs. IC50 of the same drug in the wild-type parental cell line. The different resistant sublines are indicated above the bars: A549cis1, A549cis2.5, A549cis5, A549cis7.5 and A549cis10; A549car1, A549car2.5, A549car5, A549car10, A549car15 and A549car17.5; A549ox1, A549ox2.5, A549ox3, A549ox3.5, A549ox5 and A549ox7.5; and H630ox1 and H630ox10.

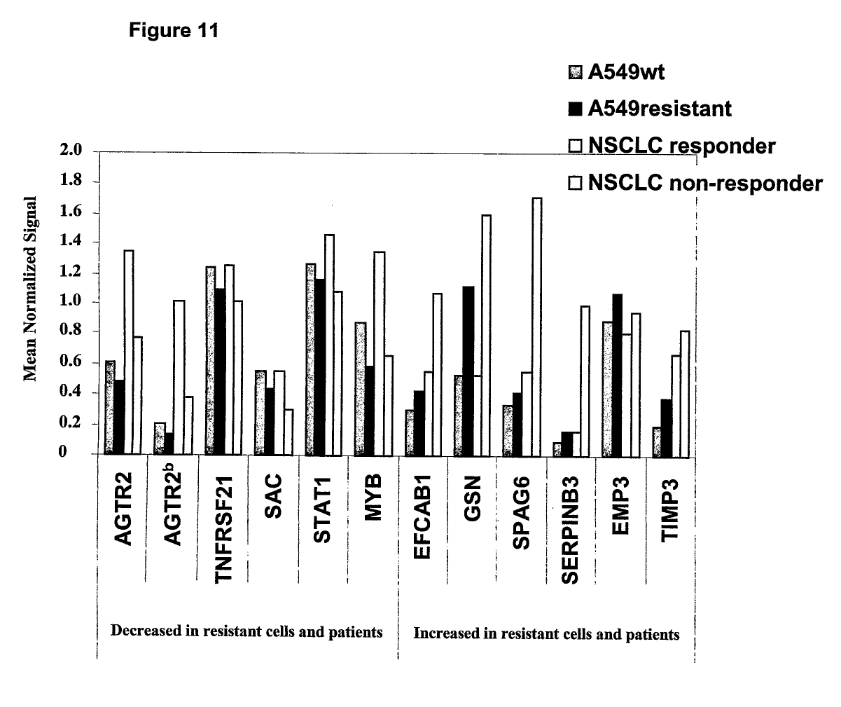

FIG. 11 shows gene expression of 11 genes consistently up- or down-regulated in platinum resistant NSCLC cells and platinum-based therapy refractory NSCLC patients. Data is mean signal measured on HGU133A microarrays and normalised in GCOSv1.4 and GeneSpringv7 as described in methods. Mean normalised signal in A549 wt (n=3) vs. A549 resistant cells (n=9: A549car2.5, car5 and car15; A549cis2.5, cis5 and cis7.5; A549ox2.5, ox3 and ox7.5) and responding (n=8) vs. non-responding platinum-treated NSCLC patients (n=8)^{ref} is shown for each gene. Probe set IDs are 222321_at (AGRT2b), 207294_at (AGTR2), 218856_at (TNFRSF21), 214547_at (SAC), 209969_s_at (STAT1), 204798_at (MYB), 220156_at (EFCAB1), 214040_s_at (GSN), 210032_s_at (SPAG6), 209720_s_at (SERPINB3), 203729_at (EMP3) and 201150_s at (TIMP3).

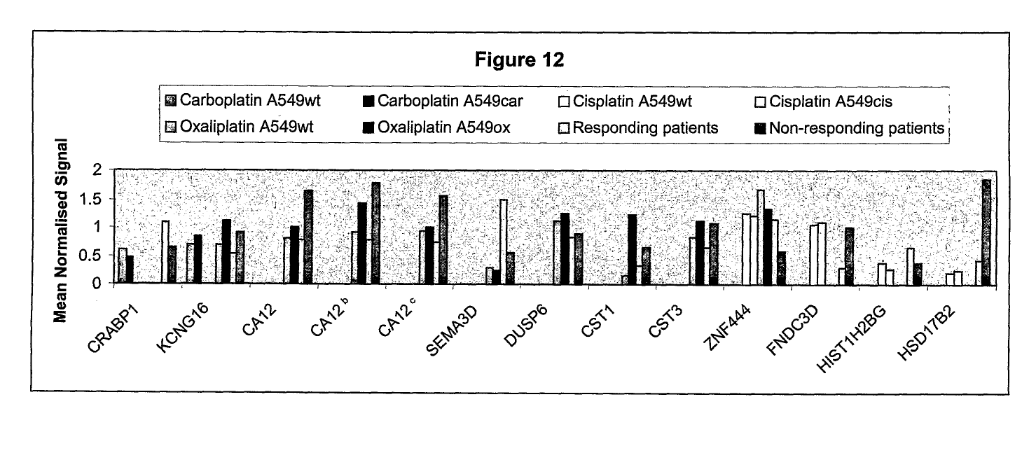

FIG. 12 shows gene expression of 11 genes consistently up- or down-regulated in cisplatin-, carboplatin- and/or oxaliplatin-resistant NSCLC cells and platinum-based therapy refractory NSCLC patients. Data is mean signal measured on HGU133A microarrays and normalised in GCOSv1.4 and GeneSpringv7 as described in methods. Mean normalised signal in A549 wt (n=3) vs. A549 carboplatin-resistant (n=3: A549car2.5, car5 and car15) cisplatin-resistant (n=3: A549cis2.5, cis5 and c is 7.5) or oxaliplatin-resistant (n=3: A549ox2.5, ox3 and ox7.5) cells and responding (n=8) vs. non-responding platinum-treated NSCLC patients (n=8)^{ref} is shown for each gene. Only the resistant subtype demonstrating an altered expression pattern that parallels that seen in non-responding NSCLC patients is shown. Probe set IDs are 205350_at (CRABP1), 219564_at (KCNG16), 215867_x_at (CA12), 214164_x_at (CA12^{b}), 210735_s_at (CA12^{c}), 215643_at (SEMA3D), 208892_s_at (DUSP6), 206224_at (CST1), 201360_at (CST3), 218707_at (ZNF444), 215910_s_at (FNDC3D, 215779_s_at (HIST1H₂BG), 204818_at (HSD17B2).

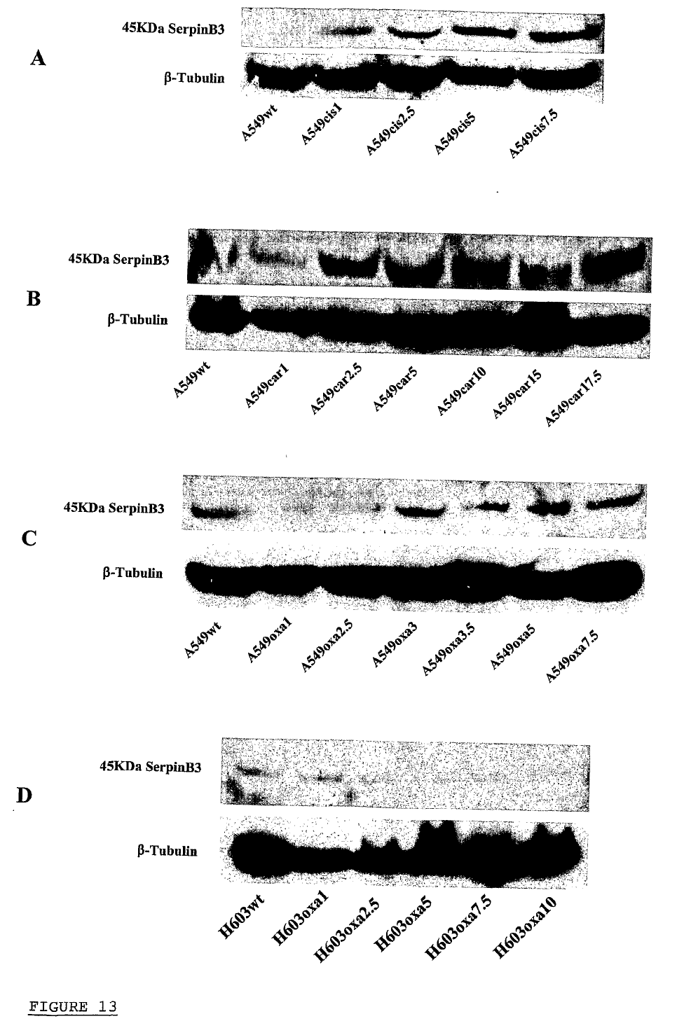

FIG. 13 shows Western analysis of serpinB3 protein expression in A549 NSCLC and H630 CRC cells. SerpinB3 expression is increased in carboplatin- (A), cisplatin- (B), and oxaliplatin- (C) resistant A549 cells compared to the parental A549 wt line. SerpinB3 protein is expressed at very low levels in H630 wt and H630 oxaliplatin-resistant cells (D). β-Tubulin is the loading control.

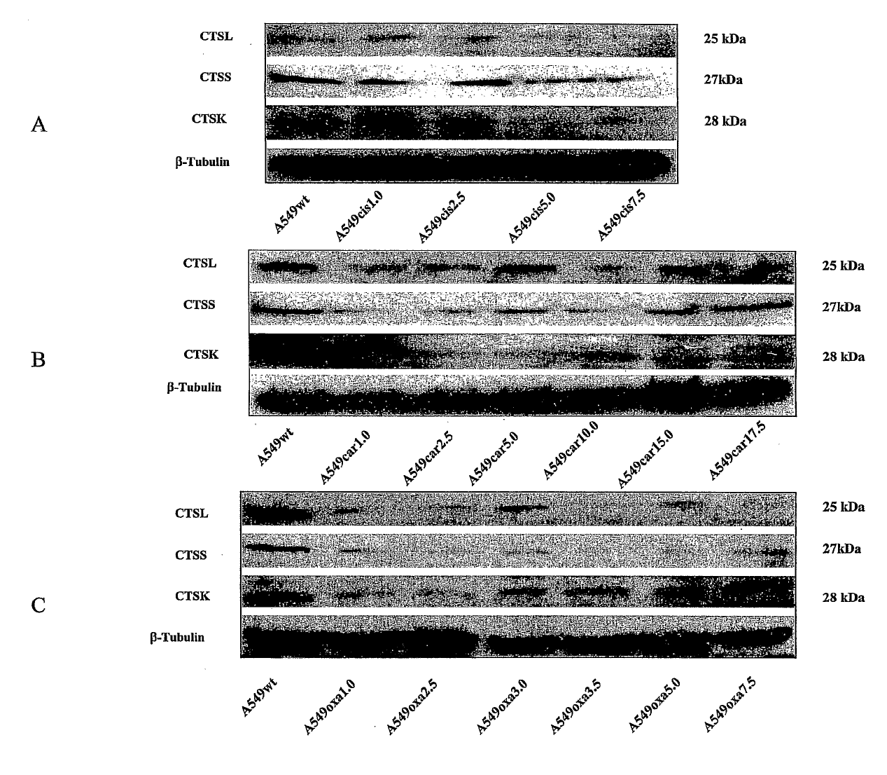

FIG. 14 shows Western analysis of serpinB3 protease targets, cathepsins L, K and S in A549 NSCLC cells. CTSL, K and S protein expression are decreased in carboplatin- (A), cisplatin- (B), and oxaliplatin- (C) resistant A549 cells compared to the parental A549 wt line. β-Tubulin is the loading control.

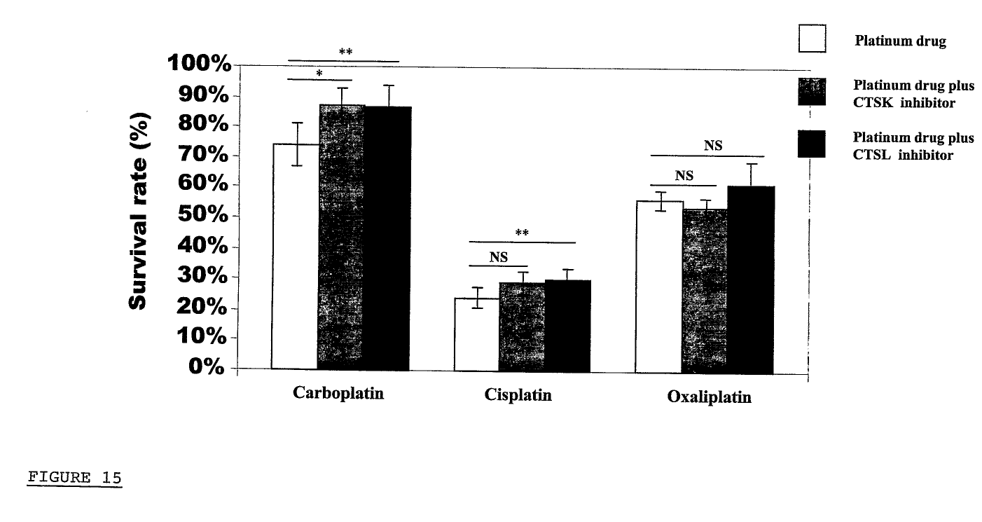

FIG. 15 shows Cytotoxicity of carboplatin (120 μM), cisplatin (13 μM) or oxaliplatin (30 μM) in A549 wt cells in the presence or absence of 1 μM CTSK inhibitor or 100 nM CTSL inhibitor. Data are expressed as percentage survival relative to untreated A549 wt cells, which was classed as 100% in MTT analysis. NS: p>0.05; *: p<0.05; **: p<0.01.

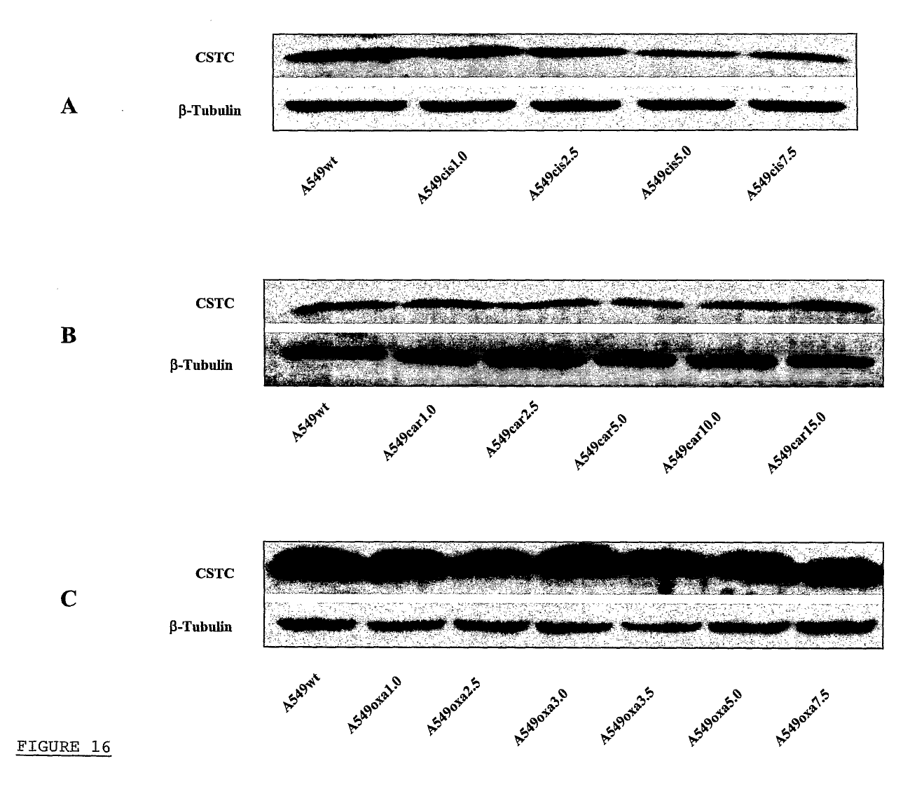

FIG. 16 shows Western analysis of cystatin C protein (13 KDa) in A549 NSCLC cells, demonstrating expression is not altered in carboplatin- (A), cisplatin- (B) or oxaliplatin- (C) resistant A549 cells compared to the parental A549 wt line. β-Tubulin is the loading control.

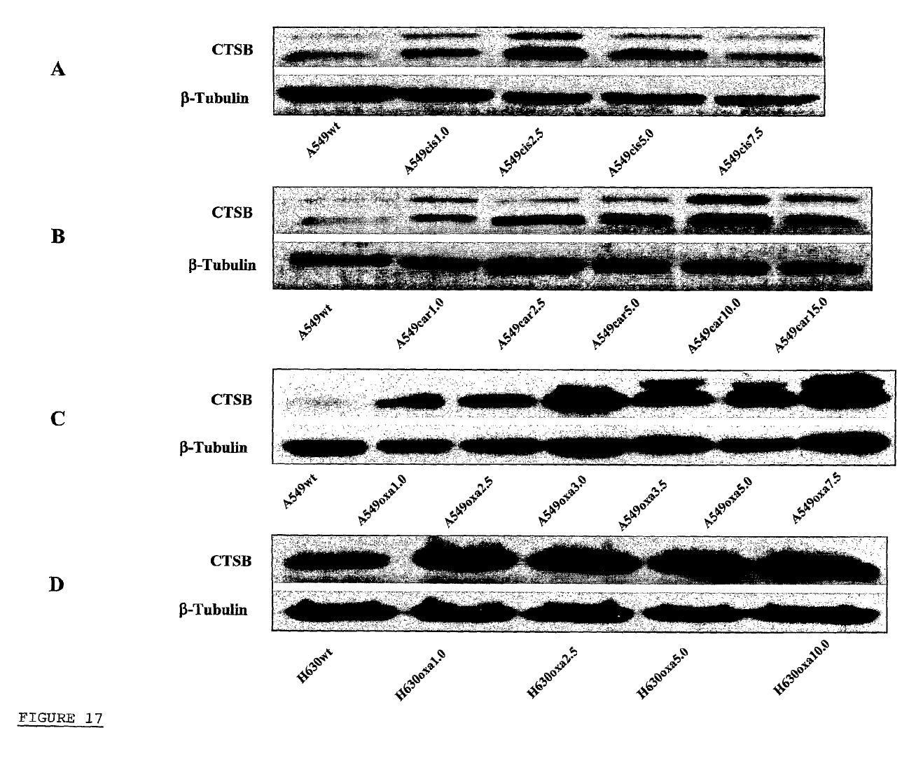

FIG. 17 shows Western analysis of cathepsin B protein in platinum-resistant NSCLC and CRC cells, demonstrating expression is strongly increased in carboplatin-(A), cisplatin- (B) or oxaliplatin- (C) resistant A549 cells and oxaliplatin-resistant H630 cells (D) compared to the parental A549 wt or H630 wt cells, respectively. β-Tubulin is the loading control. The 33 KDa single chain and 27-29 KDa double heavy chain isoforms were detected in A549 cells.

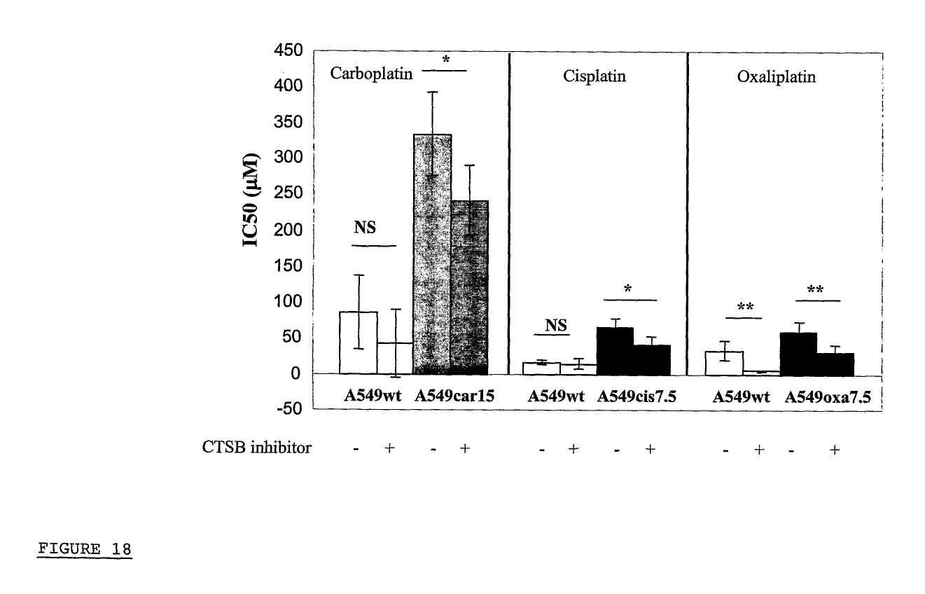

FIG. 18 shows Cytotoxicity of carboplatin, cisplatin or oxaliplatin in A549 wt or A549 platinum-resistant (A549car15, A549cis7.5 and A549ox7.5, respectively) cells, in the presence or absence of 100 μM CTSB inhibitor, CA074 methyl ester. Data are expressed as IC50 (μM) determined by MTT using a range of carboplatin (0-2 mM), cisplatin (0-500 μM) or oxaliplatin (0-500 μM) concentrations. NS: p>0.05; *: p<0.05; **: p<0.01.

### PATIENTS AND METHODS

Patient demographics and clinicopathological data. The study was performed within the guidelines and with the approval of the regional research ethics committee. All patients presented and were treated within the Departments of Oncology or Cardiothoracic Surgery at Aberdeen Royal Infirmary and full clinicopathological details are provided in the text. Pre-operative staging was with CT scan of the chest and upper abdomen and mediastinoscopy and in advanced disease with CT scan chest and upper abdomen (other investigations including CT head and isotope bone scan directed by clinical features). All stage information is clinical stage (unless otherwise indicated) according to International Union Against Cancer TMN classification of Malignant tumours, sixth edition²². Response to chemotherapy was assessed according to RECIST criteria²³. Clinicopathological information was collected prospectively. The follow-up of resected patients was by treating surgeons at regular intervals (3-12 months) for 5 years with standard radiographs and/or CT scans and minimum follow up for all patients was 10 years from surgery (median for those still alive squamous cell carcinomas=18.3 years, adenocarcinomas=15.7 years). All patients were enrolled in regional tumour registry and overall survival times from date of surgery provided by review of hospital records and public records. Chemotherapy regimens: MVP is Mitomycin C (8 mg/m²-cycles 1 and 2 only), Vinblastine (6 mg/m²) and Cisplatin (50 mg/m²) administered every 21 days; NP is Vinorelbine (30 mg/m²) and Cisplatin (80 mg/m²) administered every 21 days; Gemcitabine (1250 mg/m²) and Cisplatin (60 mg/m²) administered every 21 days; Carboplatin (AUC6) and Paclitaxel (225 mg/m²) administered every 21 days; Cisplatin (60 mg/m²) and Paclitaxel (225 mg/m²) administered every 21 days; Carboplatin (AUC 6) and docetaxel (75 mg/m²) administered every 21 days.

Specimen collection, storage and preparation for gene expression profiling. The processes and procedures for fresh tissue accrual for RNA extraction and analysis are illustrated in FIG. 1. Resection specimens were transported immediately to the laboratory in 0.9% (w/v) saline and a senior consultant pathologist (KMK) provided representative biopsies of tumour and adjacent uninvolved lung tissue, which were immediately snap frozen in liquid nitrogen and stored at −80° C. Frozen sections were cut and stained with haematoxylin and eosin to confirm histological diagnosis and determine tumour cellularity. There was no correlation between tumour cellularity and expression of any of the predictive genes subsequently identified in microarray analyses (data not shown). Extraction and purification of total RNA was performed using TRIZOL reagent (Invitrogen, Carlsbad, Calif.) and RNeasy Minikits (Qiagen, Venlo, Netherlands), respectively, according to the manufacturer's instructions. Reverse transcription of cDNA from 8 μg of total RNA (Superscript II kit, Invitrogen, Carlsbad, Calif.) and synthesis and amplification of biotin-labelled cRNA by in vitro transcription (RNA Transcript labelling kit, ENZO Diagnostics, Farmingdale, N.Y.) was performed according to the manufacturer's instructions and standard protocols (Affymetix, Santa Clara, Calif.). Quantification of total RNA and biotin-cRNA was performed by spectrophometry and the 260/280 ratio was between 1.9 and 2.2 for all samples. Quality of total RNA and cRNA was assessed using a BioAnalyser 2100 (Agilent technologies, Palo Alto, Calif.).

Gene expression profiling and data analysis. Biotin-labelled cRNA (20 μg) was fragmented at 94° C. for 35 minutes and a hybridisation cocktail was prepared from 15 μg of fragmented cRNA according to standard protocols (Affymetrix, Santa Clara, Calif.). Fragmented cRNA (5 μg) was first hybridised to Test 3 GeneChips™ to assess sample quality and then to HGU133A GeneChips™ (10 μg) for gene expression analysis. Procedures for hybridisation, washing, staining and scanning of chips were carried out according to standard protocols (Affymetrix, Santa Clara, Calif.). Initial quality control analysis and normalisation of data was performed using MASv5.0 software (Affymetrix, Santa Clara, Calif.). Subsequent filtering of data was performed using MicroDBv5.0 and DMTv3.0 (Affymetrix, Santa Clara, Calif.) and additional threshold and probabilistic filtering, supervised analysis using gene ontologies, hierarchical cluster analysis and leave-one-out cross-validation using Fishers exact t test hypergeometric probability and KNA was performed using GeneSpring v6.1 (Silicon Genetics, Redwood City, Calif.). NetAffx Analysis Center (Affymetrix, Santa Clara, Calif.) was also used in supervised analysis of the data. Gene expression signals were normalised using scaling of all probe sets to an arbitrary target signal of 100 (MASv5.0). This data was utilised for QC and generation of “detection call” and “change call” gene lists (MicroDBv3.0 and DMTv3.0, Affymetrix). Further details of these algorithms have been described previously 24 and are available from the software provider (Affymetrix, Santa Clara, Calif.). Additionally, the signal was transformed and per chip and per gene normalisation steps were performed in GeneSpring v6.1 prior to detailed analysis of the data: 1) signals <0.01 transformed to 0.01 to allow more efficient analysis of log transformed data; 2) per chip, each measurement on the array was normalised to the 50^{th} percentile of all measurements on the array and 3) per gene, each gene was normalised to its median value across all arrays in the experiment, to compare the relative gene expression changes of each gene in different samples. In the case of tumour to normal (T:N) comparisons, normalised gene expression data for individual tumours was expressed relative to the normalised gene expression in each tumours' matched uninvolved normal lung control.

Semi-quantitative RT-PCR validation of gene expression profiling data. Semi-quantitative real-time RT-PCR was performed for measurement of gene expression levels of 4 genes (TNFSF21, Cystatin C, TOMM7, GAPDH) with a broad range of both absolute expression levels and fold change between tumour and normal, in the 8 tumours in the training set and their matched normal lung controls (supplementary FIG. 1).

Real time RT-PCR was performed using the Opticon system and software and SYBR green fluorecent label, according to manufacturer's recommendations (MJ Research, Watertown, Mass.). cDNA from clinical specimens was prepared from the total RNA prepared for microarray analysis, using the Superscript-II kit according to manufacturer's instructions (Invtrogen, Carlsbad, Calif.). cDNA prepared from total RNA extracted from MCF-7 cells was used as a standard and a single batch of cDNA was used in all experiments for this purpose. 20 ng of cDNA was utilised in each PCR reaction. Each tumour and matched normal lung sample from all 8 patients in the training set utilised in the microarray analyses was included on each 96 well plate and each sample was analysed in quadruplicate. On each 96 well plate, the MCF-7 cDNA was used to generate a standard curve from the mean of triplicate wells of each concentration. The optimal Tₘ for specific and efficient amplification was identified using the gradient block on the Opticon system. The specificity of each set of primers was confirmed by running an aliquot of PCR amplified MCF7 cDNA on a 1% agrose gel, prior to real time PCR analysis. The software was utilised to calculate C_{T} values and determine the appropriate melting/read temperature, to enable accurate measurement of specific product not influenced by primer-dimers or non-specific products.

The primers used were as follows;

Cystatin C
5′-AACAAAGGCCGCCTGCTGCCTTCTC-3′
and
5′-GCAGGGCACAATGACCTTGTCGAAA-3′;
TNFRSF21
5′-AACTGAGCATTAGAAGGTACATTTG-3′
and
5′0TCAATAGGTCCAATCTGCTCTCAAG-3′
TOMM7
5′-GCTTTATCCCTCTTGTGATTTACCT-3′
and
5′-GTGAAGAGCCTTGTGCCATCCAACT-3′
GAPDH
5′-ACATGGCCTCCAAGGAGTAAGACCC-3′
and
5′GGTACTTTATTGATGGTACATGACA-3′.

A strong, highly significant correlation in gene expression measured using either technology, was observed. (Supplementary FIG. 1)

Statistical Analysis. Continuity corrected χ² or Fisher's exact test were used for binary categorical variables, Pearson χ² was used for non-binary categorical variables and Student's t-test for numerical variables. A logistic regression model was used for multivariate analysis. Kaplan-Meier and the log rank test were used for analysis of survival. Two-sided p values of <0.05 were considered significant. Levene's test was used to determine equality of variances. All analyses were performed using SPSS for Windows, version 13.0 (SPSS Inc, Chicago, Ill.).

Immunohistochemistry. All sections were obtained from archived formalin-fixed paraffin-embedded tissues obtained for routine diagnostic purposes from NSCLC patients. All cases were reviewed by a consultant pulmonary pathologist (KMK). Cases for the squamous cell cancer tissue microarray were resected between 1980 and 1990, to identify a large untreated cohort of resected NSCLC. For quality control of the SCC tissue microarray, 103 of the 193 tumours were each represented by 2 independent 1.6 nm cores and 6 normal lung cores were included.

In all cases, antigen retrieval was performed by microwaving in 10 mM citrate (pH 6.0) for 20 minutes. An autostainer (Dakocytomation, Glostrup, Denmark) was used for detection of proteins using specific primary antibodies and either the CSAII detection system (fluoroscein labelled tyrainine amplification for SerpinB3) or Chemate-Envision detection system (Cystatin C and Cathepsin B) (Dakocytomation, Glostrup, Denmark) according to the manufacturer's instructions. All sections were double scored by 2 independent investigators (KMK, RDP or PHR), who were blinded to the clinical data. Overall, >80% agreement in scoring was observed for each molecule. Scoring discrepancies were resolved by examination of sections at a double-headed microscope. Serpin B3 mouse monoclonal antibody (Santa Cruz Biotechnology, Santa Cruz, Calif.) was used at a dilution of 1:400. The sections were scored: no staining (0); >0-10% positive tumour cells (1); >10-50% positive tumour cells (2); or >50% positive tumour cells (3). All SerpinB3 positive cells demonstrated strong staining and 4 clear and distinct patterns of staining were observed, which formed the basis of the scoring system (FIG. 7). Cystatin C rabbit polyclonal antibody (Dakocytomation, Glostrup, Denmark) and mouse monoclonal cathepsin B antibody (Abcam, Cambridge, United Kingdom) were used at dilutions of 1:125 or 1:200, respectively. Due to observed variation in both the number and intensity of positive tumour cells for each protein, sections were scored for percent of positive staining tumour cells (<10% (0), 10-30% positive (1), 30-70% positive (2) and >70% positive (3)) and intensity of staining (none (0), weak (1), moderate (2) or strong (3)). The final score for cystatin C or cathepsin B was obtained by addition of the percent and intensity scores.

### Results

### EXAMPLE 1

Identification of genes highly correlated with clinical response to platinum based combination chemotherapy in NSCLC. Gene expression levels of over 22000 transcripts, in a training set of 8 consecutive patients with resectable NSCLC, who underwent 3 cycles of platinum based cytotoxic chemotherapy (4 responders and 4 non-responders) prior to surgical resection of their primary tumours, were profiled using Affymetrix HGU133A GeneChip™ oligonucleotide microarrays (table 1 and FIG. 1a). Tumour and adjacent uninvolved lung tissue samples were profiled for each patient (FIG. 1a). The flow diagram in FIG. 2 illustrates the bioinformatics analysis performed in order to identify a set of genes (n=17) whose expression was highly correlated with clinical response in the training set. Gene expression levels of the 17 genes in the predictive gene set (FIG. 2 and table 2) classified all tumours in the training set correctly according to response in hierarchical clustering (FIG. 3a) and using leave one out cross-validation.

Examination of the normalised expression of the 17 genes revealed a set of genes whose expression is highly discriminatory with regard to clinical response (table 2 and FIGS. 3a and 3b). Generally, the genes show a 4-10 fold change in their mean expression between responders and non-responders (table 2 and FIG. 4a). However there is one distinct outlier, encoding the cross class lysosomal protease inhibitor Serpin B3^{25;26} showing a 50 fold change (table 2 and FIG. 4a). Serpin B3 gene expression shows a highly significant correlation with the degree of response seen clinically on CT scan (FIG. 4b). Serpin B3 has been implicated in cancer cell line studies as a negative regulator of programmed cell death (PCD) in response to both cytotoxic drugs and radiation^{27;28}.

In order to further confirm the importance of these genes in determination of clinical response and to achieve a proof of principle that the derived gene set predicts clinical response, gene expression levels in an independent test set of 8 NSCLC primary tumour tissues, were profiled using Affymetrix HGU133A oligonucleotide microarrays (table 3 and FIG. 1b). The independent test set of NSCLC patients was not utilised in the generation of the predictive molecular signature, included patients with early stage operable disease, who received 3 cycles of platinum based chemotherapy prior to surgical resection of their tumours, and patients with metastatic disease who received up to 4 cycles of platinum based chemotherapy upon relapse (table 3). The fresh tissue used for profiling was obtained at the time of surgical resection of the primary tumour (FIG. 1b).

All of the tumours in the independent test set (8/8) clustered appropriately according to response in hierarchical clustering using standard correlation of the expression levels of the predictor genes (n=17) in the 8 training samples and each of the independent test samples (examples shown in FIGS. 5a-c).

Both clinical and pre-clinical work has suggested the importance of programmed cell death in the mechanism of action or resistance to cytotoxic drugs²⁹⁻³¹. This directed us to further investigate the role of all cell death pathways in the determination of clinical response and pathogenesis of NSCLC (FIG. 6). A global supervised analysis of PCD pathways (n=1007 probe sets; Supplementary Table 1 and FIG. 6) identified key cell death genes and pathways associated with sensitivity or resistance to PBC in NSCLC (table 4). These response-associated cell death genes included Serpin B3 (up-regulated in non-responders), which has a previously documented role as a negative regulator of cell death^{27;28} and another cross-class lysosomal protease inhibitor cystatin C (down-regulated in responders), an inhibitor of the cysteine protease, cathepsin B, which has a documented role in bid cleavage and cell death³²⁻³⁴. Gene expression levels of key genes previously reported to play a role in platinum resistance were evaluated and expression in non-responding patients was consistent with a platinum resistant phenotype (supplementary FIG. 2).

Semi-quantitative real-time PCR was performed to validate gene expression over a wide and representative range of raw signals and fold changes measured on the microarrays. Gene expression levels measured using either microarray or real-time PCR analysis were highly correlated (Supplementary FIG. 1).

### EXAMPLE 2

Immunohistochemical Analysis of Lysosomal Cysteine Protease Inhibitors. To confirm the importance of the lysosomal protease inhibitors identified from the gene expression profiling studies, the protein expression of three proteins was investigated using IHC: Serpin B3 and Cystatin C, both correlated with response in the gene expression profiling studies and Cathepsin B, the major lysosomal protease target of Cystatin C, which has a documented role in PCD³²⁻³⁴. These proteins were evaluated in patients treated with PBC. This set included pre-chemotherapy tumour tissues from 36 patients (Table 5a) and post-chemotherapy tumour tissues from 13 patients (Table 5b), and includes patients with different stages of disease (I-IV), histological subtypes (adenocarcinomas and squamous cell carcinomas), and platinum based combination chemotherapy regimens. No clinicopathological variable was significantly different between response groups (FIGS. 5a and b).

Scoring systems were derived for each protein (Serpin B3, Cystatin C and Cathepsin B), representative of the range of staining patterns seen within the full set of patients (see methods). The scoring system was designed to reflect protein levels within the tumour cells. Staining for Serpin B3 was seen exclusively within the cytoplasm of tumour cells (FIG. 7a). Staining for Cystatin C and Cathepsin B was seen within the cytoplasm and on the membrane of tumour cells, but staining within the tumour stroma was also observed (not shown). Scoring was representative of staining only within the tumour cells.

In pre-chemotherapy tumour biopsies (n=36), a significant association between Serpin B3 protein expression and response was demonstrated (p=0.045; FIG. 7b and table 6), and SerpinB3 expression in >10% of tumour cells (IHC score >1) was invariably associated with chemoresistance (FIG. 7b). In post-chemotherapy tumour biopsies (n=13), a significant association between Serpin B3 protein expression and response was also demonstrated (p=0.01; table 6). A combined IHC score designed to reflect protease inhibitory activity (Serpin B3+(Cystatin C/Cathepsin B)) revealed a highly significant relationship between clinical response to platinum based chemotherapy in both pre-chemotherapy (n=36) and post-chemotherapy (n=13) specimens (p=0.007 and p=0.021, respectively, FIG. 7c and table 6). A statistically significant increase in cathepsin B protein expression was observed following PBC (p=0.021), although paired pre-chemotherapy and post-chemotherapy tumour tissues were only available for a small subset of the NSCLC patients (n=8). No significant difference was observed in Serpin B3 or cystatin C protein expression before and after PBC in these patients.

In multivariate analysis of the pre-chemotherapy specimens (n=36), including the clinical variables sex, age (>or <70 years), smoking history, weight loss (<or >than 10%), performance status (WHO 0 vs 1), stage (early vs late), histological type (SCC vs AC), histological grade (poor vs moderate/well differentiated) and chemotherapy regimen, a combined IHC score (Serpin B3+(Cystatin C/Cathepsin B)) using a threshold cut-off of 2.0 was an independent predictor of response (OR 17.8, 95% Confidence limits 2.0-162.4, p=0.01). A combined IHC score of >2 was almost invariably associated with resistance to PBC (specificity for non-response 91%, FIG. 7c). In pre-chemotherapy biopsies, this test provides a sensitivity for response of 94% and a specificity for non-response of 91%, with an overall accuracy of 72% for prediction of outcome using combined IHC score with a threshold cut-off of 2 [≦2 (response) or >2 (non-response)] and thus provides a positive predictive value for non-response of 90%.

### EXAMPLE 3

Immunohistochemical investigation of SerpinB3 in chemotherapy-naive NSCLC patients. To investigate the role of Serpin B3 in NSCLC pathogenesis and prognosis we examined its expression by immunohistochemistry in 176 lung squamous cell carcinomas (tissue microarray with 193 tumours, 17 cores lost during staining procedure (8.8%), leaving 176 tumours for evaluation, see methods) and 75 stage, age and grade matched adenocarcinomas (clinicopathological details provided in table 7). Matched primary tumour and tumour containing regional lymph nodes were available for 64 patients (SCC n=29 and AC n=35). No patients received treatment with chemotherapy at any stage of their management thereby allowing an assessment of the purely prognostic impact of Serpin B3, independent of any effects of therapy.

Overall Serpin B3 staining was more commonly positive in SCC than AC (p<0.0001, table 8). There was no significant association between Serpin B3 protein expression and any clinicopathological variable (histological type, UTCC stage, grade, gender, smoking history, age).

As described earlier, a Serpin B3 IHC score of >1 (>10% of tumour cells positive; FIG. 7b) was invariably associated with chemoresistance and applying this as a cut-off in the chemotherapy-naïve NSCLC patients, reveals contrasting prognostic impact of Serpin B3 in SCC and AC (FIGS. 8a and 8b).

In adenocarcinomas, high Serpin B3 protein expression (IHC score >1) is a poor prognostic factor (FIG. 8a). In multivariate analysis with the clinical variables stage, grade, gender, smoking history and age (>or <70 years), high Serpin B3 protein expression (IHC score >1) is an independent prognostic marker of 5 year survival in NSCLC patients with AC (n=75; HR for death at 5 years=2.09 (95% CI 1.03-4.72), p=0.042; FIG. 8a).

In SCC, a contrasting prognostic impact is seen. High Serpin B3 protein expression in SCC (IHC score >1) is a good prognostic factor. In multivariate analysis of SCC with the clinical variables stage, grade, gender, smoking history, age (>or <70 years), Serpin B3 protein expression is an independent good prognostic factor for 5 year survival (HR for death at 5 years=0.43 (95% CI 0.18-0.93); p=0.049, FIG. 8b). However, the prognostic impact varies with nodal status, so that in N0 and N1 tumours high Serpin B3 protein expression remains associated with good prognosis (median survival 77 months versus 25 months, p=0.027, FIG. 8c), while in N2 tumours it is a poor prognostic factor (median survival 3 versus 16 months, p=0.017, FIG. 8d).

The distinct prognostic impacts of Serpin B3 expression in AC and SCC may be explained by different putative roles for Serpin B3, including negative regulation of both invasion and metastasis³⁵⁻³⁷ and cell death^{27;28}. Consistent with this hypothesis, in the matched primary AC and metastatic regional lymph nodes there was no significant change in Serpin B3 expression in tumour cells (FIG. 9b), suggesting that Serpin B3 does not inhibit invasion and metastases in adenocarcinomas of the lung. This is in contrast to down-regulation of Serpin B3 in metastatic lymph nodes of SCC (p=0.003; FIG. 9a) that suggests a role in invasion and metastases in this histological sub-type.

### Discussion

To provide new insight for the treatment of NSCLC we have performed a global molecular characterisation of clinical response to platinum based chemotherapy in NSCLC patients. In the development of a predictive test for clinical response and supervised analysis of cell death pathways using gene expression profiling, we have identified genes that are strongly associated with clinical response. The importance of key biomarkers has been confirmed in an independent set of patients using gene expression profiling and immunohistochemical analysis of protein expression in PBC treated NSCLC patients. The correlation of these markers with response to cytotoxic therapy in NSCLC patients, persists independent of diverse clinical and pathological parameters, including pre- and post-chemotherapy tissues, different platinum based regimens, different histological types of NSCLC and different clinical stages of disease. Together the data provide an important “proof of principle” that global gene expression profiling can be used to derive a molecular signature capable of predicting individual patient response to systemic treatment in NSCLC. Additionally, our approach has identified novel molecules and pathways that may be important mechanistic determinants of clinical response or resistance, disease pathogenesis and therapy independent prognosis, thereby providing targets for further investigation as novel therapeutics.

The cross class lysosomal protease inhibitor Serpin B3 has been identified in our studies as a biomarker that has both predictive value for response to platinum based combination chemotherapy and also independent prognostic value in untreated patients with resected NSCLC. mRNA and protein expression levels for Serpin B3 each demonstrated a strong correlation with clinical response in PBC treated NSCLC patients. Immunohistochemical measurement of protein expression levels of Serpin B3 with another lysosomal protease inhibitor identified in this study, cystatin C and its main physiological target, cathepsin B, which has a documented role in PCD³²⁻³⁴, provides an independent predictor of response to platinum based chemotherapy (HR 17.8, 95% CI 2.0-162.4, p=0.01). The sensitivity for response prediction (combined IHC score <2, FIG. 7c) is good (94%), but specificity is limited (64%), although the specificity for non-responding patients (combined IHC score >2, FIG. 7c) is high (91%). This test therefore provides an accuracy of 72% and suggests that over 50% of patients who are unlikely to benefit from PBC may be prospectively identified using the protein expression levels of these 3 proteins alone (Serpin B3+Cystatin C/Cathepsin B), potentially allowing first line treatment with platinum-independent or novel therapies. The highly accurate performance of the 17 gene predictive set (table 2) with the independent test set of NSCLC patients (examples shown in FIG. 5) illustrates that sensitivity for non-responding patients may be improved by incorporation of further markers, but additionally demonstrates the undoubtedly multifaceted nature of clinical chemoresistance in NSCLC. Nevertheless, the importance of these two lysosomal cysteine cathepsin protease inhibitors is suggested and represents the first report of a putative role of lysosomal proteases and their inhibitors in response or resistance to cytotoxic therapy in NSCLC patients, and suggests a role of for the recently described pathway of lysosomal cathepsin protease mediated cell death. The likely physiological role of SerpinB3 is to protect against “leaked” lysosomal proteases and it has also been shown to be a negative regulator of PCD in tumour cell lines in response to cytotoxic drugs and ionising radiation^{27,28}.

Cathepsin B has been reported to have prognostic value in NSCLC patients⁴⁵, which may be related to its role in invasion and metastases. Although cathepsin B protein expression alone was not correlated with response to PBC treated NSCLC patients in our study, analysis of this protease with both its inhibitor cystatin C and Serpin B3 was strongly correlated with response to PBC in NSCLC patients, suggesting it may also have a role in cytotoxicity of PBC in NSCLC patients. Cathepsin B has been reported to mediate caspase-independent cell death in response to cytotoxics in NSCLC cell lines³³.

The IHC data presented here shows that expression of Serpin B3 protein is detected in both adenocarcinomas and squamous cell carcinomas of the lung. Although Serpin B3 expression is associated with SCC at various sites including lung, this is the first detailed report of Serpin B3 expression in adenocarcinomas of the lung. The contrasting prognostic impact of Serpin B3 expression in AC and SCC, may relate to a dual pathogenic role of Serpin B3 in NSCLC of different histological types, which is consistent with the suggested molecular and cellular functions of Serpin B3 and one of its protease substrates cathepsin L^{27;28;35-37}. In patients with resected tumours, particularly in the absence of any chemotherapy, the major determinant of long term cancer-specific survival is expected to be micro-metastatic disease which would account for the association of high Serpin B3 expression as a good prognostic factor in N0/N1 SCC through a putative role in negative regulation of invasion and metastases. Similarly, its association with poor prognosis in N2 disease may relate to a predominant role of Serpin B3 as a negative regulator of cell death in these advanced stage NSCLC patients. The data from matched primary SCC and metastatic regional lymph nodes supports a role for Serpin B3 in the inhibition of invasion and metastasis, as Serpin B3 expression is reduced in the metastatic nodes compared to the primary SCC (p=0.003).

In AC, the data from matched primary and lymph nodes suggest that Serpin B3 does not have a role in invasion and metastasis as expression levels in the nodal tumour cells are not significantly different from that in the paired primary tumour. High Serpin B3 expression is a poor prognostic factor in AC and potentially Serpin B3 may primarily function as a negative regulator of cell death in this histological type.

The association of strong Serpin B3 protein expression, which is invariably associated with resistance to Pt based chemotherapy, with good prognosis in untreated early stage disease SCC may provide a useful biomarker, for example, in the selection of patients who would or would not benefit from adjuvant chemotherapy. Strongly Serpin B3 positive stage 1B and IIA SCCs are likely to be chemoresistant, but our data suggests a good prognosis in these patients following resection in the absence of any therapy. This is therefore not likely to represent a group that would benefit from adjuvant chemotherapy, but under current practice many oncologists would treat all such patients with adjuvant chemotherapy. The association of high Serpin B3 expression with chemoresistance in all PBC treated NSCLC patients and poor prognosis in specific histological and clinical subsets of NSCLC patients, including all AC and N2 SCC may also provide a useful biomarker. Strongly Serpin B3 positive AC and N2 SCC are unlikely to benefit from Pt based chemotherapy and our data suggests this population has a poor prognosis if untreated. Therefore alternative therapeutic approaches would be indicated in these patients who could accordingly avoid the unnecessary toxicity of Pt based chemotherapy from which they are unlikely to benefit.

Importantly, the data presented here has implications for novel therapeutic approaches in NSCLC treatment. The mechanisms by which these lysosomal proteases and their inhibitors may mediate chemoresistance remain unresolved but there are several possibilities. Without wishing to be bound by theory Serpin B3 is a cytosolic protein, which can be secreted, Cystatin C is a secreted protein which is functional extracellularly, but that can be re-internalised into the endo-lysosomal compartment and may have as yet uncharacterised intracellular roles⁴⁶. Localisation of both cathespin B and cystatin C has been reported on the surface of tumour cells and in juxtanuclear vessels⁴⁷. Alternatively, cystatin C may play an important role in preventing necrotic tumour cell death following the release of lysosomal proteases, especially cathepsin B, in chemotherapy treated NSCLC patients. Necrosis is certainly observed in chemotherapy treated lung cancers⁴⁸ Serpin B3 may prevent programmed cell death occurring by the recently described lysosomal pathway where a triggering event is the release of lysosomal cathepsin cysteine proteases into the cytosol, an event which may be induced by cytotoxic chemotherapy^{29;32;49-50}.

Overall our study has identified predictive and prognostic biomarkers and has suggested the importance to clinical response of a previously unsuspected pathway and group of proteins. The potential involvement of lysosomal proteases and their inhibitors in clinical response to cytotoxic therapy, cell death, invasion and metastasis mean that they are intriguing targets for novel therapeutics, particularly Serpin B3. We suggest that these molecules and this pathway warrant further mechanistic and clinical investigation and may hold promise as a source of novel therapeutic targets for this devastating disease.

### Further Materials and Methods

### Cell Lines

The human NSCLC cell line A549 [from ATCC; CCL-185] was maintained in F12-K and RPMI 1640 (supplemented with 10% foetal bovine serum (FBS), 100 U/ml penicillin, and 100 μg/ml streptomycin at 37° C., 5% CO₂) in an incubator.

The A549 cisplatin-, carboplatin- and oxaliplatin-cells were derived from the A549_{WT} cell lines, by maintaining the cells in increasing concentrations of the relevant drug. The lowest concentration was 1 nM, this was slowly increased to a final concentration of 10 μM, 17.5 μM and 7.5 μM respectively. This generated cisplatin-(A549cis1, A549cis2.5, A549cis5, A549cis7.5 and A549cis10), carboplatin-(A549car1, A549car2.5, A549car5, A549car10, A549car15 and A549car17.5) and oxaliplatin-(A549oxa2.5, A549oxa3, A549ox3.5, A549ox5 and A549oxa7.5) resistant A549 cell lines. The cells were exposed to each drug concentration for at least a 4-week period and allowed to recover in drug-free medium for a minimum of 2 weeks, before culture in a higher concentration of drug.

### Cytotoxicity Assay

Cytotoxicity of each platinum drug in each cell line was determined by MTT (3-(4,5-dimethylthiazole-2-yl)-2,5-diphenyl tetrazolium bromide, Sigma) assay. Cells were seeded onto 96-well plates at a density of 2-3×10³ cells per well and cultured for 24 hours. Then cells were incubated with relevant concentrations of carboplatin, cisplatin or oxaliplatin for 72 hours. After treatment, 30 μl of MTT (5 mg/ml) in PBS was incubated with cells in a 96-well plate for 4 h at 37° C. Subsequently, the medium containing MTT was removed, and 200 μl of DMSO was added to each well and shaken for 15 minutes. Spectrophotometric absorbance of each well was measured at 560 nm and 630 nm on a microplate reader (Bio-Rad, model 3550). GraphPad Prism 2.01 was used to calculate the IC50 of each platinum drug in different cell lines.

### Inhibitors

CA-074 Methyl ester (Sigma) was used to inhibit cathepsin B and caspase inhibitor I: (Z-VAD (OMe)-FMK, Calbiochem, UK) to inhibit caspases. Cathepsin L and K selective inhibitors (Cathepsin L inhibitor IV: Z-FF-FMK and Cathepsin K inhibitor I: 1,3-Bis (N-carbobenzoyloxy-L-leucyl) amino acetone, respectively (Calbiochem, UK)) and non-selective broad range cathepsin inhibitor (E-64d; (2S, 3S)-trans-epoxysuccinyl-L-leucylamido-3-methylbutane ethyl ester (Sigma, UK) were used to inhibit cysteine proteases.

### Western Blotting

Cells were harvested from 75 cm² flask after growth in the absence of platinum drug for 2 weeks. Cells were washed 3 times with PBS and lysed using RIPA buffer [150 mM NaCl, 10 mM Tris (pH 7.5), 5 mM EDTA, 1.0% Triton X-100, 0.1% SDS, 1% deoxycholate, protease inhibitors (Complete; Roche Diagnostics Corp) and 100 μM sodium orthovanadate]. The concentration of protein was determined by Bradford method (Bio-Rad Detection Reagent) and 50-150 μg of protein/well were loaded onto a denaturing polyacrylamide gel (10% or 15%) with a 4% stacking gel, which was electrophoresed for 2 hours at 100V and transferred to polyvinylidene difluoride membrane (New England Nuclear Life Sciences, Boston, Mass.). The membrane was stained with Ponceau S to check transfer efficiency, and was blocked with 5% skimmed milk (Marvel) in PBS-T [0.1% (w/v) Tween 20 in PBS (pH 7.4)] for 2 hour at room temperature (RT). Membranes were incubated with relevant primary antibodies in 5% milk PBS-T overnight at 4° C. or 2 hours at RT (SCCA1, Santa Cruz Biotechnology, CA; CTSB, CTSS, CTSL, Abcam, UK; CTSK, Calbiochem; CSTC, Dakocytomation, Denmark). Horseradish peroxidise-linked secondary antibodies (1:5000, Amersham Pharmacia Biotech, Amersham, UK) were incubated for 30 minutes at RT. The blots were developed using the ECL-plus detection kit (Amersham, UK). Anti-tubulin monoclonal antibody (Sigma-Aldrich, UK) was used to confirm equal loading.

### Gene Expression Profiling

Total RNA was extracted from A549 parental and resistant daughter lines with TRIzol (Invitrogen, Carlsbad, Calif.) and was purified using RNeasy minikits (Qiagen, Venlo, the Netherlands), following the manufacturer's instructions. For cDNA synthesis, 8 μg of total RNA was converted by reverse transcription to cDNA and to biotin-labelled cRNA by IVT following the manufactuer's instructions (One cycle transcript labelling kit, Affymetrix, Santa Clara, Calif.). Labelled amplified cRNA was purified using RNeasy minikits (Qiagen) and fragmented cRNA was hybridised to HGU133A GeneChips™ (Affymetrix, Santa Clara, Calif.) for gene expression analysis. Staining, washing and scanning of microarrays were performed on a FS400 fluidics station and GCS3000 scanner according to standard protocols. For quality control, total RNA and labelled-cRNA were analysed by spectrophometry and the 260:280 ratio was between 1.9 and 2.2 for all samples. Bioanalyser 2100 (Agilent Technologies, Palo Alto, Calif.) and Test 3 GeneChip™ (Affymetrix, Santa Clara, Calif.) analyses were used to assess the quality of total RNA and cRNA. All Actin and GAPDH 3′:5′ ratios were <3.

### Data Analysis

Initial quality control analysis and normalization of data were performed using GCOS v1.2. Threshold filtering of data was performed using MicroDB v5.0, DMTv3.0 (Affymetrix, Santa Clara, Calif.), and additional threshold and probabilistic filtering, supervised analyses using gene ontologies and hierarchical clustering were performed using GeneSpring 7.2 (Agilent Technologies, CA). NetAffx Analysis Center (http://www.affymetrix.com/analysis/index.affx; Affymetrix) was used in supervised analysis of the data. Gene expression signals were normalized using scaling of all probe sets to an arbitrary target signal of 100 (GCOS v1.2). These data were utilised for QC and generation of ‘change call’ gene lists. Additionally, data were imported into GeneSpring v7.2 (Agilent Technologies, **) and values of less than 0.01 were transformed to 0.01 to optimise analysis of log transformed data, each measurement on the array was normalised to the 50^{th} percentile of all measurements on the array to allow a per chip normalization and each gene was normalised to its median value across all arrays in the study. The resulting expression levels were thus centred around 1 and thus allows comparison of the relative expression changes of each gene in different samples.

### Results

### Platinum Chemoresistance in A549 Cell Lines

Oxaliplatin-, cisplatin- and carboplatin-resistant A549 NSCLC cells were generated as described (see materials and methods). Increasing levels of resistance to the relevant platinum drug were observed in cells, following exposure to increasing concentrations of drug (FIG. 10). Comparison of IC50 values demonstrated that cisplatin- and carboplatin-exposed A549 cells were approximately 3-fold more resistant than the parental A549 line, after exposure to a maximum concentration of 7.5 μM cisplatin or 15 μM carboplatin, respectively and this level of resistance remained unchanged at higher drug concentrations (10 μM and 17.5 μM, respectively). In contrast, resistance to oxaliplatin was more difficult to develop in A549 cells but once achieved, higher levels of resistance were obtained at lower concentrations of drug (3-fold at 3 μM and 5.3-fold at 7.5 μM) compared to the resistance levels observed in cisplatin- or carboplatin-resistant A549 cells.

Cisplatin-resistant (A549cis7.5; IC50 3.3- vs. 3.4-fold) and carboplatin-resistant (A549car15; IC50 3.02- vs. 3.05-fold) A549 cells were equally resistant to cisplatin or carboplatin, respectively, compared to the A549 parental line. In contrast, interestingly, cisplatin- and carboplatin-resistant cells had increased sensitivity to oxaliplatin (IC50 0.54 and 0.47-fold, respectively) compared to the A549 parental line. Oxaliplatin-resistant cells (A549ox7.5) had a moderate level of resistance to cisplatin or carboplatin (IC50 1.8- or 1.5-fold, respectively) compared to A549 wt cells.

These data are consistent with the clinical observation of oxaliplatin response in cisplatin/carboplatin-refractory NSCLC and colorectal cancer patients.

### Functional Relevance of Platinum-Chemoresistance Biomarkers

Studies of the NSCL tumour transcriptome as described above, identified a set of 27 genes correlated with resistance to platinum-based therapy in NSCLC patients (Tables 2 and 4). To evaluate the potential functional relevance of each of these platinum-resistance biomarkers, we evaluated their gene expression in sensitive A549 wt and cisplatin-, carboplatin- and oxaliplatin-resistant A549 daughter cells (FIG. 11).

Of the 27 genes we previously reported to be correlated with platinum response or resistance in NSCLC patients, 11 of these [AGTR2, EFCAB1, TNFRSF21, SAC, GSN, SPAG6, STAT1, SerpinB3, MYB, EMP3 and TIMP3] demonstrated the same pattern of altered gene expression as was observed in studies of NSCLC patients. Thus EFCAB1, gelsolin, SPAG6, SerpinB3, EMP3 and TIMP3 were up-regulated in non-responding patients and platinum-resistant cell lines compared to responding patients or platinum-sensitive A549 wt cells, respectively (FIG. 11). In contrast, AGRT2, TNFRSF21, SAC, STAT1 and MYB were reduced in non-responding patients and platinum-resistant cell lines compared to responding patients or platinum sensitive A549 wt cells, respectively (FIG. 11). These data suggest that these biomarkers may be involved in mediating the chemoresistance phenotype rather than simply being biomarkers of resistance without biological effect. Additionally, 11 of the 27 genes demonstrated the same pattern of altered gene expression in one or two of the platinum resistant lines as was observed in non-responding NSCLC patients: CRABP1 (carboplatin-resistant); H1ST1H2BG, FNDC3D, HSD17B2 (cisplatin-resistant); CA12, SEMA3D, DUSP6, CST1, CST3 (oxaliplatin-resistant); ZNF444 (cisplatin- and oxaliplatin-resistant); and KCNG16 (carboplatin- and oxaliplatin-resistant) (FIG. 12).

Thus of the 27 genes identified to be correlated with response or resistance to platinum-based therapy in NSCLC patients, 22 of these demonstrated a similar altered pattern of expression in non-responding patients and carboplatin-, cisplatin- and/or oxaliplatin-resistant NSCLC cell lines. These data suggest that the majority of these novel biomarkers may contribute to the resistance or responsive phenotypes rather than simply acting as diagnostic markers in these patients.

SerpinB3 mRNA and Protein Expression in Platinum-Resistant Non-Small Cell Lung Cancer Cells.

In the studies of NSCLC patients, serpinB3 was identified as an outlier with a mean fold increase in mRNA levels of approximately 8-fold in non-responding compared to responding platinum-based chemotherapy-treated NSCLC patients (Table 2 and FIG. 4a). Additionally, mRNA levels were strongly correlated with degree of tumour reduction measured by CT scan of NSCL tumours (R=−0.978, p<0.0001; FIG. 4b) and high expression of serpinB3 protein was invariably associated with resistance to platinum-based therapy in NSCLC patients. Thus we evaluated serpinB3 expression in our cisplatin-, carboplatin- and oxaliplatin-resistant NSCLC cells.

Analysis of gene expression on DNA microarrays demonstrated that serpinB3 mRNA has a mean fold increase of 8.0- and 1.7-fold in early (A549cis2.5, A549car2.5 and A549ox2.5) and intermediate (A549cis5, A549car5 and A549ox7.5) platinum-resistant A549 cells, respectively, compared to sensitive A549 wt cells (n=3). In contrast, serpinB3 mRNA expression is down-regulated (Mean −2.6-fold) in late platinum-resistant A549 cells (A549cis7.5, A549car15 and A549ox7.5) compared to the parental line (not shown).

These gene expression changes were confirmed at the protein level by western analysis, where SerpinB3 protein was increased in cisplatin-, carboplatin and oxaliplatin-resistant A549 cells (FIG. 13). The attenuation observed in mRNA levels in late resistance was not observed at the protein level in platinum-resistant A549 cells. Thus, in contrast to mRNA levels, serpinB3 protein over-expressed at all stages of cisplatin-, carboplatin- and oxaliplatin-resistance in A549 NSCLC cells (FIG. 13).

These data suggest that increased expression of serpinB3 mRNA and protein, which as identified herein to be a marker of resistance to platinum-based therapies in NSCLC patients, may be a functional event with biological consequences.

### SerpinB3 Targets, Cathepsins L, K and S in Platinum-Resistant NSCLC Cells.

Protein expression levels of the serpinB3 cysteine protease targets, cathepsins L, S and K, were evaluated by western analysis in sensitive and cisplatin-, carboplatin- and oxaliplatin-resistant A549 cells (FIG. 14). Expression of CTS L, K and S proteins was reduced in cisplatin-, carboplatin- and oxaliplatin-resistant cells compared to the parental A549 wt cells. The antibodies against cathepsins L and S recognise both the unprocessed inactive zymogen and the mature active forms of the proteins. Only the 37 KDa CTS S prepropeptide zymogen was detected in A549 wt cells and this was reduced in platinum-resistant cells (FIG. 14). In contrast, only the mature 27 KDa CTS L protein was detected in A549 wt cells and expression levels were reduced in platinum-resistant cells (FIG. 14). The antibody against CTS K only recognises the 28 KDa mature form of the CTS K protein and this was detected at low levels in A549 wt cells and levels were reduced in A549 platinum-resistant cells, although this reduced expression was attenuated in late-oxaliplatin resistant cells. Cathepsins L, K and S cysteine proteases were down-regulated as a late resistance mechanism in the cisplatin-resistant A549 cells but this was an early event in the carboplatin and oxaliplatin-resistant cells.

Gene expression of cathepsins L, S and K was not altered in drug resistant cells compared to the parental line (data not shown). This suggests that reduced expression of these proteases in platinum-resistant NSCLC cells is a post-transcriptional event.

Our data demonstrate that protein expression of the lysosomal cysteine proteases, cathepsins L, K and S is reduced, while expression of serpinB3 protein, the cross-class inhibitor of these cysteine proteases, is induced in platinum-resistant NSCLC cells. These data suggest that blockage of this lysosomal protease pathway is a critical mechanism of resistance to cisplatin, carboplatin and oxaliplatin in NSCLC cells.

### Inhibition of Cathepsins L or K Reduces Sensitivity to Platinums in NSCLC Cells.

To further evaluate the relationship between the cathepsins L or K expression and platinum drug resistance in NSCLC cells, we inhibited their activity using specific cysteine protease inhibitors. CTS K or CTSL inhibition significantly reduced the cytotoxic effect of carboplatin in A549 wt cells (<0.05 and p<0.01, respectively; FIG. 15). In addition, inhibition of CTSL, but not CTSK, significantly decreased the cytotoxicity of cisplatin in A549 wt cells (p<0.01) (FIG. 15). In contrast, inhibition of either CTSL or CTSL did not alter the cytotoxicity of oxaliplatin in A549 wt cells (FIG. 15.).

### Cystatin C and Cathepsin B Expression in Platinum-Resistant Cells.

As discussed above, reduced expression of cystatin C (CST3), which like serpinB3 is a lysosomal cysteine protease inhibitor, was correlated with response to platinum-based therapy in NSCLC (FIG. 7, Tables 4 and 6). CST3 is an inhibitor of cathepsin B, which has been shown to mediate caspase-dependent and caspase-independent cell death. It was demonstrated that high expression of serpinB3 and cystatin C, relative to its target protease cathepsin B, was almost invariably associated with lack of response to platinum-based chemotherapy in NSCLC patients (FIG. 7c). An immunohistochemical test of serpinB3, cystatin C and cathepsin B protein levels, allowed classification of response vs. non-response to platinum-based therapy with 72% accuracy and provided a positive predictive value of 90%, for identification of refractory patients.

CST3 mRNA was increased in A549 oxaliplatin resistant cells compared to the parental lines (FIG. 12) but was reduced in A549 carboplatin and cisplatin resistant cells (not shown). Additionally, in contrast to our NSCLC patient data, in our non-small cell lung cancer cell line models, cystatin C protein expression was not altered between cisplatin-, carboplatin- or oxaliplatin-resistant and sensitive parental A549 cells (FIG. 16).

Surprisingly, expression of cathepsin B protein was strongly increased in platinum-resistant NSCLC cells compared to the parental line (FIG. 17). This induced expression was an early event in acquired resistance to each of the 3 platinum drugs, but was attenuated in late cisplatin and carboplatin resistance. However, levels of CTS B protein continued to increase in late oxaliplatin resistance. Both the 33 kDa active single chain pre-peptide generated by proteolytic removal of the pro-fragment and the active double-chain form (27-29 kDa), which is generated from the pre-peptide and is the most active form of the protease, were detected in our platinum-resistant NSCLC cells.

These data suggest a putative anti-apoptotic role for CTS B in the platinum-resistant NSCLC cell lines and are in contrast with the earlier hypothesised role for CTS B in response to platinum-based therapy in NSCLC patients.

### Inhibition of Cathepsin B Increases Sensitivity of NSCLC Cells to Platinum Drugs

Inhibition of cathepsin B significantly increased the sensitivity of A549 wt to oxaliplatin (p<0.01), but the effects of cisplatin or carboplatin on these NSCLC cells were not changed in response to CTS B inhibition (FIG. 18). In contrast, the cytotoxicity of carboplatin, cisplatin or oxaliplatin in carboplatin (p=0.03), cisplatin (p=0.02) or oxaliplatin-resistant (p=0.01) A549 daughter cells, was significantly increased by 35.1%, 44.5% and 32.4%, respectively when CTSB activity was inhibited (FIG. 18).

Broad range inhibition of cysteine proteases using E64d (12.5 μM) also enhanced the cytotoxicity of carboplatin, cisplatin and oxaliplatin in A549 wt cells (not shown). In contrast, inhibition of caspases did not alter the sensitivity of wild-type or resistant A549 NSCLC to these platinum drugs (data not shown).

Thus our platinum-resistant cell lines, while providing a good model for analysis of serpinB3 and its targets in NSCLC cells, appear not to reflect the in vivo cystatin C/cathepsin B pathway in refractory NSCL tumours.

### REFERENCES

1. IARC. GLOBOCAN 2000: Cancer Incidence, Mortality and Prevelance Worldwide, Version 1.0, IARC Cancerbase No5. Ved. IARC Press, Lyon, France; 2001.
2. Petty R D, Nicolson M C, Kerr K M, et al. Gene expression profiling in non-small cell lung cancer: from molecular mechanisms to clinical application. Clin Cancer Res 10: 3237-3248, 2004.
3. Balmain A, Gray J, Ponder B. The genetics and genomics of cancer. Nat Genet. 33 Suppl: 238-244, 2003.
4. Cree I A, Neale M H, Myatt N E, et al. Heterogeneity of chemosensitivity of metastatic cutaneous melanoma. Anticancer Drugs 10: 437-444, 1999.
5. Mercer S J, Somers S S, Knight L A, et al. Heterogeneity of chemosensitivity of esophageal and gastric carcinoma. Anticancer Drugs 14: 397-403, 2003.
6. Whitehouse P A, Knight L A, Di Nicolantonio F, et al. Heterogeneity of chemosensitivity of colorectal adenocarcinoma determined by a modified ex vivo ATP-tumor chemosensitivity assay (ATP-TCA). Anticancer Drugs 14: 369-375, 2003.
7. Paik S. Incorporating genomics into the cancer clinical trial process. Semin Oncol 28: 305-309, 2001.
8. Beer D G, Kardia S L, Huang C C, et al. Gene-expression profiles predict survival of patients with lung adenocarcinoma. Nat Med 8: 816-824, 2002.
9. van de Vijver M J, He Y D, Vant Veer U, et al. A gene-expression signature as a predictor of survival in breast cancer. N EngI J Med 347: 1999-2009, 2002.
10. Bhattachaijee A, Richards W G, Staunton j, et al. Classification of human lung carcinomas by mRNA expression profiling reveals distinct adenocarcinoma subclasses. Proc Natl Acad Sci US A 98: 13790-13795, 2001.
11. Nielsen T O, West R B, Linn S C, et al. Molecular characterisation of soft tissue tumours: a gene expression study. Lancet 359: 1301-1307, 2002.
12. Sorlie T, Perou C M, Tibshirani R, et al. Gene expression patterns of breast carcinomas distinguish tumor subclasses with clinical implications. Proc Natl Acad Sci USA 98: 10869-10874, 2001.
13. Meyerson M, Carbone D. Genomic and Proteomic Profiling of Lung Cancers: Lung Cancer Classification in the Age of Targeted Therapy. Journal of Clinical Oncology 23: 3219-3226, 2005.
14. Ligon K L, Alberta J A, Kho A T, et al. The oligodendroglial lineage marker OLIG2 is universally expressed in diffuse gliomas. J Neuropathol Exp Neurol 63: 499-509, 2004.
15. Ntzani E E, loannidis J P. Predictive ability of DNA microarrays for cancer outcomes and correlates: an empirical assessment. Lancet 362: 1439-1444, 2003.
16. Nun C L, Mani D R, Betensky R A, et al. Gene expression-based classification of malignant gliomas correlates better with survival than histological classification. Cancer Res 63: 1602-1607, 2003.
17. Lee Y F, John M, Falconer A, et al. A gene expression signature associated with metastatic outcome in human leiomyosarcomas. Cancer Res 64: 7201-7204, 2004.
18. Staunton J E, Slonim D K, Coller H A, et al. Chemosensitivity prediction by transcriptional profiling. Proc Natl Acad Sci USA 98: 10787-10792, 2001.
19. Dan S, Tsunoda T, Kitahara O, et al. An integrated database of chemosensitivity to 55 anticancer drugs and gene expression profiles of 39 human cancer cell lines. Cancer Res 62: 1139-1147, 2002.
20. Zembutsu H, Ohnishi Y, Tsunoda T, et al. Genome-wide cDNA microarray screening to correlate gene expression profiles with sensitivity of 85 human cancer xenografts to anticancer drugs. Cancer Res 62: 518-527, 2002
21. Chang B D, Swift M E, Shen M, et al. Molecular determinants of terminal growth arrest induced in tumor cells by a chemotherapeutic agent. Proc Natl Acad Sci USA 99: 389-394, 2002.
22. L. H. Sobin (Editor), Ch. W. E. TNM Classification of Malignant Tumours, 6th Edition. 2002. 2005. Indianapolis, USA, John Wiley and Sons.
23. Therasse P, Arbuck S G, Eisenhauer E A, et al. New guidelines to evaluate the response to treatment in solid tumors. European Organization for Research and Treatment of Cancer, National Cancer Institute of the United States, National Cancer Institute of Canada. J Natl Cancer Inst 92: 205-216, 2000.
24. Wang W, Cassidy J, O'Brien V, et al. Mechanistic and predictive profiling of 5-Fluorouracil resistance in human cancer cells. Cancer Res 64: 8167-8176, 2004.
25. Schick C, Pemberton P A, Shi G P, et al. Cross-class inhibition of the cysteine proteinases cathepsins K, L, and S by the serpin squamous cell carcinoma antigen 1: a kinetic analysis. Biochemistry 37: 5258-5266, 1998.
26. Masumoto K, Sakata Y, Arima K, et al. Inhibitory mechanism of a cross-class serpin, the squamous cell carcinoma antigen 1. J Biol Chem 278: 45296-45304, 2003.
27. Suminami Y, Nagashima S, Vujanovic N L, et al. Inhibition of apoptosis in human tumour cells by the tumour-associated serpin, SCC antigen-1. Br J Cancer 82: 981-989, 2000.
28. Murakami A, Suminami Y, Hirakawa H, et al. Squamous cell carcinoma antigen suppresses radiation-induced cell death. Br J Cancer 84: 851-858, 2001.
29. Leist M, Jaattela M. Four deaths and a funeral: from caspases to alternative mechanisms. Nat Rev Mol Cell Biol 2: 589-598, 2001.
30. Blagosklonny MV. Prospective strategies to enforce selectively cell death in cancer cells. Oncogene 23: 2967-2975, 2004.
31. Shah M A, Schwartz G K. Cell cycle-mediated drug resistance: an emerging concept in cancer therapy. Clin Cancer Res 7: 2168-2181, 2001.
32. Leist M, Jaattela M. Triggering of apoptosis by cathepsins. Cell Death Differ 8: 324-326, 2001.
33. Broker L E, Huisman C, Span S W, et al. Cathepsin B Mediates Caspase-Independent Cell Death Induced by Microtubule Stabilizing Agents in Non-Small Cell Lung Cancer Cells. Cancer Research 64, 27-30, 2004.
34. Cirman, T.; Oresic, K.; Mazovec, G. D, et al. Selective disruption of lysosomes in HeLa cells triggers apoptosis mediated by cleavage of bid by multiple papain-like lysosomal cathepsins. Journal of Biological Chemistry 279: 3578-3587, 2004.
35. Iwasaki M, Nishikawa A, Akutagawa N, et al. E1AF/PEA3 reduces the invasiveness of SiHa cervical cancer cells by activating serine proteinase inhibitor squamous cell carcinoma antigen. Exp Cell Res 299: 525-532, 2004.
36. Lakka S S, Gondi C S, Yanamandra N, et al. Inhibition of cathepsin B and MMP-9 gene expression in glioblastoma cell line via RNA interference reduces tumor cell invasion, tumor growth and angiogenesis. Oncogene 23: 4681-4689, 2004.
37. Joyce J A, Baruch A, Chehade K, et al. Cathepsin cysteine proteases are effectors of invasive growth and angiogenesis during multistage tumorigenesis. Cancer Cell 5:443-453, 2004.
38. Kato H, Torigoe T. Radioimmunoassay for tumor antigen of human cervical squamous cell carcinoma. Cancer 40: 1621-1628, 1977.
39. Cataltepe S, Schick C, Luke C J, et al. Development of specific monoclonal antibodies and a sensitive discriminatory immunoassay for the circulating tumor markers SCCA1 and SCCA2. Clin Chim Acta 295: 107-127, 2000.
40. Stenman J, Hedstrom J, Grenman R, et al. Relative levels of SCCA2 and SCCA1 mRNA in primary tumors predicts recurrent disease in squamous cell cancer of the head and neck. Tnt J Cancer 95: 39-43, 2001.
41. Pontisso P, Calabrese F, Benvegnu L, et al. Overexpression of squamous cell carcinoma antigen variants in hepatocellular carcinoma. Br J Cancer 90: 833-837, 2004.
42. Vassilakopoulos T, Troupis T, Sotiropoulou C, et al. Diagnostic and prognostic significance of squamous cell carcinoma antigen in non-small cell lung cancer. Lung Cancer 32: 137-144, 2001.
43. Diez M, Torres A, Maestro M L, et al. Prediction of survival and recurrence by serum and cytosolic levels of CEA, CA12S and SCC antigens in resectable non-small-cell lung cancer. British Journal of Cancer 73: 1248-1254, 1996.
44. Pastor A.; Menendez R.; Cremades M J, et al. Diagnostic value of SCC, CEA and CYFRA 21.1 in lung cancer: A Bayesian analysis. European Respiratory Journal 10: 603-609, 1997.
45. Kayser K Richter N Hufuagl P et al. Expression, proliferation activity and clinical significance of cathepsin B and cathepsin L in operated lung cancer. Anticancer Res. 23: 2767-2772, 2003.
46. Merz G S, Benedikz E, Schwenk V, et al. Human cystatin C forms an inactive dimer during intracellular trafficking in transfected CHO cells. J Cell Physiol 173: 423-432, 1997.
47. Calkins C C, Sameni M, Koblinski J, et al. Differential Localization of Cysteine Protease Inhibitors and a Target Cysteine Protease, Cathepsin B, by Immuno-Confocal Microscopy. Journal of Histochemistry and Cytochemistry 46: 745-752, 1998.
48. Liu-Jarin X, Stoopler M B, Raftopoulos H, et al. Histologic assessment of non-small cell lung carcinoma after neoadjuvant therapy. Mod Pathol 16: 1102-1108, 2003.
49. Fehrenbacher N, Gyrd-Hansen M, Poulsen B, et al. Sensitization to the lysosomal cell death pathway upon immortalization and transformation. Cancer Res 64: 5301-5310, 2004.
50. Fehrenbacher N, Jaattela M. Lysosomes as targets for cancer therapy. Cancer Res 65: 2993-2995, 2005.
51. Raymond E, Chaney S G, Taamma A. Cvitkovic E. Oxaliplatin: a review of preclinical and clinical studies. Ann Oncol. 1998 October: 9(10): 1053-71.

SUPPLEMENTARY TABLE 1
List of 1007 cell death genes from
Affymetrix HGU133A genechip

| | | Chromosomal |
|---|---|---|
| Probe Set ID | Gene Symbol | Location |
| 1729_at | TRADD | 16q22 |
| 1861_at | BAD | 11q13.1 |
| 200004_at | EIF4G2 | 11p15 |
| 200046_at | DAD1 | 14q11-q12 |
| 200063_s_at | NPM1 | 5q35 |
| 200071_at | SMNDC1 | 10q23 |
| 200602_at | APP | 21q21.3 |
| 200607_s_at | RAD21 | 8q24 |
| 200608_s_at | RAD21 | 8q24 |
| 200661_at | PPGB | 20q13.1 |
| 200695_at | PPP2R1A | 19q13.41 |
| 200696_s_at | GSN | 9q33 |
| 200704_at | LITAF | 16p13.3-p12 |
| 200706_s_at | LITAF | 16p13.3-p12 |
| 200724_at | RPL10 | xq28 |
| 200725_x_at | RPL10 | xq28 |
| 200766_at | CTSD | 11p15.5 |
| 200787_s_at | PEA15 | 1q21.1 |
| 200788_s_at | PEA15 | 1q21.1 |
| 200796_s_at | MCL1 | 1q21 |
| 200797_s_at | MCL1 | 1q21 |
| 200798_x_at | MCL1 | 1q21 |
| 200803_s_at | TEGT | 12q12-q13 |
| 200804_at | TEGT | 12q12-q13 |
| 200830_at | PSMD2 | 3q27.1 |
| 200837_at | BCAP31 | xq28 |
| 200838_at | CTSB | 8p22 |
| 200839_s_at | CTSB | 8p22 |
| 200887_s_at | STAT1 | 2q32.2 |
| 200959_at | FUS | 16p11.2 |
| 200976_s_at | TAX1BP1 | 7p15 |
| 200977_s_at | TAX1BP1 | 7p15 |
| 201020_at | YWHAH | 22q12.3 |
| 201083_s_at | BCLAF1 | 6q22-q23 |
| 201084_s_at | BCLAF1 | 6q22-q23 |
| 201085_s_at | SON | 21q22.11 |
| 201086_x_at | SON | 21q22.11 |
| 201095_at | DAP | 5p15.2 |
| 201101_s_at | BCLAF1 | 6q22-q23 |
| 201105_at | LGALS1 | 22q13.1 |
| 201111_at | CSE1L | 20q13 |
| 201112_s_at | CSE1L | 20q13 |
| 201147_s_at | TIMP3 | 22q12.3 |
| 201148_s_at | TIMP3 | 22q12.3 |
| 201149_s_at | TIMP3 | 22q12.3 |
| 201150_s_at | TIMP3 | 22q12.3 |
| 201201_at | CSTB | 21q22.3 |
| 201207_at | TNFAIP1 | 17q22-q23 |
| 201208_s_at | TNFAIP1 | 17q22-q23 |
| 201209_at | HDAC1 | 1p34 |
| 201244_s_at | RAF1 | 3p25 |
| 201324_at | EMP1 | 12p12.3 |
| 201325_s_at | EMP1 | 12p12.3 |
| 201360_at | CST3 | 20p11.21 |
| 201370_s_at | CUL3 | 2q36.3 |
| 201371_s_at | CUL3 | 2q36.3 |
| 201372_s_at | CUL3 | 2q36.3 |
| 201391_at | TRAP1 | 16p13.3 |
| 201423_s_at | CUL4A | 13q34 |
| 201424_s_at | CUL4A | 13q34 |
| 201446_s_at | TIA1 | 2p13 |
| 201447_at | TIA1 | 2p13 |
| 201448_at | TIA1 | 2p13 |
| 201449_at | TIA1 | 2p13 |
| 201450_s_at | TIA1 | 2p13 |
| 201464_x_at | JUN | 1p32-p31 |
| 201465_s_at | JUN | 1p32-p31 |
| 201466_s_at | JUN | 1p32-p31 |
| 201467_s_at | NQO1 | 16q22.1 |
| 201487_at | CTSC | 11q14.1-q14.3 |
| 201502_s_at | NFKBIA | 14q13 |
| 201587_s_at | IRAK1 | xq28 |
| 201588_at | TXNL1 | 18q21.2 |
| 201628_s_at | RRAGA | 9p22.1 |
| 201631_s_at | IER3 | 6p21.3 |
| 201635_s_at | FXR1 | 3q28 |
| 201636_at | — | — |
| 201637_s_at | FXR1 | 3q28 |
| 201686_x_at | API5 | 11p12-q12 |
| 201687_s_at | API5 | 11p12-q12 |
| 201710_at | MYBL2 | 20q13.1 |
| 201715_s_at | ACIN1 | 14q11.2 |
| 201739_at | SGK | 6q23 |
| 201743_at | CD14 | 5q31.1 |
| 201746_at | TP53 | 17p13.1 |
| 201763_s_at | DAXX | 6p21.3 |
| 201783_s_at | — | — |
| 201819_at | SCARB1 | 12q24.31 |
| 201844_s_at | RYBP | 3p13 |
| 201845_s_at | — | — |
| 201846_s_at | RYBP | 3p13 |
| 201848_s_at | BNIP3 | 10q26.3 |
| 201849_at | BNIP3 | 10q26.3 |
| 201850_at | CAPG | 2cen-q24 |
| 201897_s_at | CKS1B | 1q21.2 |
| 201965_s_at | KIAA0625 | 9q34.13 |
| 202014_at | PPP1R15A | 19q13.2 |
| 202023_at | EFNA1 | 1q21-q22 |
| 202035_s_at | SFRP1 | 8p12-p11.1 |
| 202036_s_at | SFRP1 | 8p12-p11.1 |
| 202037_s_at | SFRP1 | 8p12-p11.1 |
| 202039_at | MYO18A; TIAF1 | 17q11.2 |
| 202049_s_at | ZNF262 | 1p32-p34 |
| 202050_s_at | ZNF262 | 1p32-p34 |
| 202051_s_at | ZNF262 | 1p32-p34 |
| 202073_at | OPTN | 10p13 |
| 202074_s_at | OPTN | 10p13 |
| 202076_at | BIRC2 | 11q22 |
| 202086_at | MX1 | 21q22.3 |
| 202087_s_at | CTSL | 9q21-q22 |
| 202094_at | BIRC5 | 17q25 |
| 202095_s_at | BIRC5 | 17q25 |
| 202116_at | DPF2 | 11q13 |
| 202123_s_at | ABL1 | 9q34.1 |
| 202156_s_at | CUGBP2 | 10p13 |
| 202157_s_at | CUGBP2 | 10p13 |
| 202158_s_at | CUGBP2 | 10p13 |
| 202168_at | TAF9 | 5q11.2-q13.1 |
| 202176_at | ERCC3 | 2q21 |
| 202178_at | PRKCZ | 1p36.33-p36.2 |
| 202221_s_at | EP300 | 22q13.2 |
| 202239_at | PARP4 | 13q11 |
| 202253_s_at | DNM2 | 19p13.2 |
| 202268_s_at | APPBP1 | 16q22 |
| 202284_s_at | CDKN1A | 6p21.2 |
| 202295_s_at | CTSH | 15q24-q25 |
| 202316_x_at | UBE4B | 1p36.3 |
| 202317_s_at | UBE4B | 1p36.3 |
| 202387_at | BAG1 | 9p12 |
| 202389_s_at | HD | 4p16.3 |
| 202390_s_at | HD | 4p16.3 |
| 202405_at | TIAL1 | 10q |
| 202406_s_at | TIAL1 | 10q |
| 202431_s_at | MYC | 8q24.12-q24.13 |
| 202443_x_at | NOTCH2 | 1p13-p11 |
| 202445_s_at | NOTCH2 | 1p13-p11 |
| 202450_s_at | CTSK | 1q21 |
| 202468_s_at | CTNNAL1 | 9q31.2 |
| 202480_s_at | DEDD | 1q23.3 |
| 202509_s_at | TNFAIP2 | 14q32 |
| 202510_s_at | TNFAIP2 | 14q32 |
| 202511_s_at | APG5L | 6q21 |
| 202512_s_at | APG5L | 6q21 |
| 202535_at | FADD | 11q13.3 |
| 202643_s_at | TNFAIP3 | 6q23 |
| 202644_s_at | TNFAIP3 | 6q23 |
| 202676_x_at | FASTK | 7q35 |
| 202687_s_at | TNFSF10 | 3q26 |
| 202688_at | TNFSF10 | 3q26 |
| 202693_s_at | STK17A | 7p12-p14 |
| 202694_at | STK17A | 7p12-p14 |
| 202695_s_at | STK17A | 7p12-p14 |
| 202723_s_at | FOXO1A | 13q14.1 |
| 202724_s_at | FOXO1A | 13q14.1 |
| 202730_s_at | PDCD4 | 10q24 |
| 202731_at | PDCD4 | 10q24 |
| 202761_s_at | SYNE2 | 14q23.2 |
| 202763_at | CASP3 | 4q34 |
| 202790_at | CLDN7 | 17p13 |
| 202803_s_at | ITGB2 | 21q22.3 |
| 202820_at | AHR | 7p15 |
| 202871_at | TRAF4 | 17q11-q12 |
| 202883_s_at | PPP2R1B | 11q23.2 |
| 202884_s_at | PPP2R1B | 11q23.2 |
| 202885_s_at | PPP2R1B | 11q23.2 |
| 202886_s_at | PPP2R1B | 11q23.2 |
| 202893_at | UNC13B | 9p12-p11 |
| 2028_s_at | E2F1 | 20q11.2 |
| 202901_x_at | CTSS | 1q21 |
| 202902_s_at | CTSS | 1q21 |
| 202921_s_at | ANK2 | 4q25-q27 |
| 202980_s_at | SIAH1 | 16q12 |
| 202981_x_at | SIAH1 | 16q12 |
| 202984_s_at | BAG5 | 14q32.32 |
| 202985_s_at | BAG5 | 14q32.32 |
| 202992_at | C7 | 5p13 |
| 203005_at | LTBR | 12p13 |
| 203063_at | PPM1F | 22q11.22 |
| 203078_at | CUL2 | 10p11.21 |
| 203079_s_at | CUL2 | 10p11.21 |
| 203084_at | TGFB1 | 19q13.1 |
| 203085_s_at | TGFB1 | 19q13.1 |
| 203089_s_at | PRSS25 | 2p12 |
| 203110_at | PTK2B | 8p21.1 |
| 203111_s_at | PTK2B | 8p21.1 |
| 203120_at | TP53BP2 | 1q42.1 |
| 203139_at | DAPK1 | 9q34.1 |
| 203187_at | DOCK1 | 10q26.13-q26.3 |
| 203265_s_at | MAP2K4 | 17p11.2 |
| 203266_s_at | MAP2K4 | 17p11.2 |
| 203277_at | DFFA | 1p36.3-p36.2 |
| 203372_s_at | SOCS2 | 12q |
| 203373_at | SOCS2 | 12q |
| 203381_s_at | APOE | 19q13.2 |
| 203382_s_at | APOE | 19q13.2 |
| 203414_at | MMD | 17q |
| 203415_at | PDCD6 | 5pter-p15.2 |
| 203460_s_at | PSEN1 | 14q24.3 |
| 203489_at | SIVA | 14q32.33 |
| 203508_at | TNFRSF1B | 1p36.3-p36.2 |
| 203528_at | SEMA4D | 9q22-q31 |
| 203531_at | CUL5 | 11q22-q23 |
| 203532_x_at | CUL5 | 11q22-q23 |
| 203533_s_at | CUL5 | 11q22-q23 |
| 203618_at | FAIM2 | 12q13 |
| 203619_s_at | FAIM2 | 12q13 |
| 203627_at | IGF1R | 15q26.3 |
| 203628_at | IGF1R | 15q26.3 |
| 203657_s_at | CTSF | 11q13 |
| 203684_s_at | BCL2 | 18q21.3 |
| 203685_at | BCL2 | 18q21.3 |
| 203725_at | GADD45A | 1p31.2-p31.1 |
| 203728_at | BAK1 | 6p21.3 |
| 203729_at | EMP3 | 19q13.3 |
| 203758_at | CTSO | 4q31-q32 |
| 203804_s_at | CROP | 17q21.33 |
| 203836_s_at | MAP3K5 | 6q22.33 |
| 203837_at | MAP3K5 | 6q22.33 |
| 203844_at | VHL | 3p26-p25 |
| 203890_s_at | DAPK3 | 19p13.3 |
| 203891_s_at | DAPK3 | 19p13.3 |
| 203893_at | TAF9 | 5q11.2-q13.1 |
| 203928_x_at | MAPT | 17q21.1 |
| 203929_s_at | MAPT | 17q21.1 |
| 203930_s_at | MAPT | 17q21.1 |
| 203948_s_at | MPO | 17q23.1 |
| 203949_at | MPO | 17q23.1 |
| 203984_s_at | CASP9 | 1p36.3-p36.1 |
| 203989_x_at | F2R | 5q13 |
| 204004_at | PAWR | 12q21 |
| 204005_s_at | PAWR | 12q21 |
| 204025_s_at | PDCD2 | 6q27 |
| 204064_at | THOC1 | 18p11.32 |
| 204068_at | STK3 | 8q22.2 |
| 204113_at | CUGBP1 | 11p11 |
| 204121_at | GADD45G | 9q22.1-q22.2 |
| 204131_s_at | FOXO3A | 6q21 |
| 204132_s_at | FOXO3A | 6q21 |
| 204141_at | TUBB | 6p25 |
| 204164_at | SIPA1 | 11q13 |
| 204211_x_at | PRKR | 2p22-p21 |
| 204235_s_at | GULP1 | 2q32.3-q33 |
| 204237_at | GULP1 | 2q32.3-q33 |
| 204261_s_at | PSEN2 | 1q31-q42 |
| 204262_s_at | PSEN2 | 1q31-q42 |
| 204274_at | EBAG9 | 8q23 |
| 204278_s_at | EBAG9 | 8q23 |
| 204352_at | TRAF5 | 1q32 |
| 204413_at | TRAF2 | 9q34 |
| 204466_s_at | SNCA | 4q21 |
| 204467_s_at | SNCA | 4q21 |
| 204482_at | CLDN5 | 22q11.21 |
| 204493_at | BID | 22q11.1 |
| 204513_s_at | ELMO1 | 7p14.1 |
| 204531_s_at | BRCA1 | 17q21 |
| 204614_at | SERPINB2 | 18q21.3 |
| 204687_at | DKFZP564O0823 | 4q13.3-q21.3 |
| 204777_s_at | MAL | 2cen-q13 |
| 204780_s_at | TNFRSF6 | 10q24.1 |
| 204781_s_at | TNFRSF6 | 10q24.1 |
| 204813_at | MAPK10 | 4q22.1-q23 |
| 204817_at | ESPL1 | 12q |
| 204831_at | CDK8 | 13q12 |
| 204833_at | APG12L | 5q21-q22 |
| 204859_s_at | APAF1 | 12q23 |
| 204860_s_at | BIRC1 | 5q13.1 |
| 204861_s_at | BIRC1 | 5q13.1 |
| 204862_s_at | NME3 | 16q13 |
| 204877_s_at | TAOK2 | 16p11.2 |
| 204878_s_at | TAOK2 | 16p11.2 |
| 204924_at | TLR2 | 4q32 |
| 204926_at | INHBA | 7p15-p13 |
| 204930_s_at | BNIP1 | 5q33-q34 |
| 204932_at | TNFRSF11B | 8q24 |
| 204933_s_at | TNFRSF11B | 8q24 |
| 204947_at | E2F1 | 20q11.2 |
| 204950_at | CARD8 | 19q13.32 |
| 204971_at | CSTA | 3q21 |
| 204975_at | EMP2 | 16p13.2 |
| 204986_s_at | TAOK2 | 16p11.2 |
| 205013_s_at | ADORA2A | 22q11.23 |
| 205050_s_at | MAPK8IP2 | 22q13.33 |
| 205067_at | IL1B | 2q14 |
| 205084_at | BCAP29 | 7q22-q31 |
| 205153_s_at | TNFRSF5 | 20q12-q13.2 |
| 205176_s_at | ITGB3BP | 1p31.3 |
| 205192_at | MAP3K14 | 17q21 |
| 205214_at | STK17B | 2q32.3 |
| 205263_at | BCL10 | 1p22 |
| 205385_at | — | — |
| 205386_s_at | MDM2 | 12q14.3-q15 |
| 205387_s_at | CGB; CGB7; CGB5 | 19q13.32 |
| 205389_s_at | ANK1 | 8p11.1 |
| 205390_s_at | ANK1 | 8p11.1 |
| 205391_x_at | ANK1 | 8p11.1 |
| 205409_at | FOSL2 | 2p23.3 |
| 205411_at | STK4 | 20q11.2-q13.2 |
| 205456_at | CD3E | 11q23 |
| 205467_at | CASP10 | 2q33-q34 |
| 205481_at | ADORA1 | 1q32.1 |
| 205486_at | TESK2 | 1p32 |
| 205488_at | GZMA | 5q11-q12 |
| 205500_at | C5 | 9q33-q34 |
| 205504_at | BTK | xq21.33-q22 |
| 205512_s_at | PDCD8 | xq25-q26 |
| 205554_s_at | DNASE1L3 | 3p21.1-3p14.3 |
| 205598_at | TRIP | 3p21.31 |
| 205599_at | TRAF1 | 9q33-q34 |
| 205611_at | TNFSF12 | 17p13 |
| 205641_s_at | TRADD | 16q22 |
| 205653_at | CTSG | 14q11.2 |
| 205655_at | MDM4 | 1q32 |
| 205681_at | BCL2A1 | 15q24.3 |
| 205692_s_at | CD38 | 4p15 |
| 205745_x_at | ADAM17 | 2p25 |
| 205746_s_at | ADAM17 | 2p25 |
| 205754_at | F2 | 11p11-q12 |
| 205759_s_at | SULT2B1 | 19q13.3 |
| 205780_at | BIK | 22q13.31 |
| 205818_at | DBC1 | 9q32-q33 |
| 205831_at | CD2 | 1p13 |
| 205848_at | GAS2 | 11p14.3-p15.2 |
| 205851_at | NME6 | 3p21 |
| 205858_at | NGFR | 17q21-q22 |
| 205859_at | LY86 | 6p25.1 |
| 205927_s_at | CTSE | 1q31 |
| 205928_at | ZNF443 | 19p13.2 |
| 205963_s_at | DNAJA3 | 16p13.3 |
| 205986_at | AATK | 17q25.3 |
| 206011_at | CASP1 | 11q23 |
| 206025_s_at | TNFAIP6 | 2q23.3 |
| 206026_s_at | TNFAIP6 | 2q23.3 |
| 206044_s_at | BRAF | 7q34 |
| 206054_at | KNG1 | 3q27 |
| 206092_x_at | RTEL1 | 20q13.3 |
| 206150_at | TNFRSF7 | 12p13 |
| 206157_at | PTX3 | 3q25 |
| 206189_at | UNC5C | 4q21-q23 |
| 206197_at | NME5 | 5q31 |
| 206217_at | EDA | xq12-q13.1 |
| 206222_at | TNFRSF10C | 8p22-p21 |
| 206224_at | CST1 | 20p11.21 |
| 206248_at | PRKCE | 2p21 |
| 206259_at | PROC | 2q13-q14 |
| 206286_s_at | TDGF1 | 3p21.31 |
| 206292_s_at | SULT2A1 | 19q13.3 |
| 206293_at | SULT2A1 | 19q13.3 |
| 206295_at | IL18 | 11q22.2-q22.3 |
| 206305_s_at | C8A | 1p32 |
| 206324_s_at | DAPK2 | 15q22.31 |
| 206341_at | IL2RA | 10p15-p14 |
| 206346_at | PRLR | 5p14-p13 |
| 206359_at | SOCS3 | 17q25.3 |
| 206360_s_at | SOCS3 | 17q25.3 |
| 206362_x_at | MAP3K10 | 19q13.2 |
| 206385_s_at | ANK3 | 10q21 |
| 206399_x_at | CACNA1A | 19p13.2-p13.1 |
| 206400_at | LGALS7 | 19q13.2 |
| 206401_s_at | MAPT | 17q21.1 |
| 206444_at | PDE1B | 12q13 |
| 206467_x_at | TNFRSF6B; RTEL1 | 20q13.3 |
| 206508_at | TNFSF7 | 19p13 |
| 206531_at | DPF1 | 19q13.13-q13.2 |
| 206536_s_at | BIRC4 | xq25 |
| 206537_at | — | — |
| 206545_at | CD28 | 2q33 |
| 206556_at | CLUL1 | 18p11.32 |
| 206569_at | IL24 | 1q32 |
| 206595_at | CST6 | 11q13 |
| 206641_at | TNFRSF17 | 16p13.1 |
| 206656_s_at | C20orf3 | 20p11.22-p11.21 |
| 206665_s_at | BCL2L1 | 20q11.21 |
| 206666_at | GZMK | 5q11-q12 |
| 206680_at | CD5L | 1q21-q23 |
| 206687_s_at | PTPN6 | 12p13 |
| 206706_at | NTF3 | 12p13 |
| 206714_at | ALOX15B | 17p13.1 |
| 206724_at | CBX4 | 17q25.3 |
| 206727_at | C9 | 5p14-p12 |
| 206729_at | TNFRSF8 | 1p36 |
| 206752_s_at | DFFB | 1p36.3 |
| 206804_at | CD3G | 11q23 |
| 206863_x_at | — | — |
| 206864_s_at | HRK | 12q24.22 |
| 206865_at | HRK | 12q24.22 |
| 206879_s_at | NRG2 | 5q23-q33 |
| 206907_at | TNFSF9 | 19p13.3 |
| 206923_at | PRKCA | 17q22-q23.2 |
| 206939_at | DCC | 18q21.3 |
| 206975_at | LTA | 6p21.3 |
| 206977_at | PTH | 11p15.3-p15.1 |
| 206979_at | C8B | 1p32 |
| 206994_at | CST4 | 20p11.21 |
| 207002_s_at | PLAGL1 | 6q24-q25 |
| 207004_at | BCL2 | 18q21.3 |
| 207005_s_at | BCL2 | 18q21.3 |
| 207037_at | TNFRSF11A | 18q22.1 |
| 207061_at | ERN1 | 17q24.2 |
| 207062_at | IAPP | 12p12.3-p12.1 |
| 207075_at | CIAS1 | 1q44 |
| 207087_x_at | ANK1 | 8p11.1 |
| 207113_s_at | TNF | 6p21.3 |
| 207163_s_at | AKT1 | 14q32.32 |
| 207180_s_at | HTATIP2 | 11p15.1 |
| 207181_s_at | CASP7 | 10q25 |
| 207198_s_at | LIMS1 | 2q12.3-q13 |
| 207216_at | TNFSF8 | 9q33 |
| 207293_s_at | AGTR2 | xq22-q23 |
| 207294_at | AGTR2 | xq22-q23 |
| 207339_s_at | LTB | 6p21.3 |
| 207382_at | TP73L | 3q27-q29 |
| 207384_at | PGLYRP1 | 19q13.2-q13.3 |
| 207388_s_at | PTGES | 9q34.3 |
| 207426_s_at | TNFSF4 | 1q25 |
| 207428_x_at | CDC2L1 | 1p36 |
| 207433_at | IL10 | 1q31-q32 |
| 207460_at | GZMM | 19p13.3 |
| 207468_s_at | SFRP5 | 10q24.1 |
| 207500_at | CASP5 | 11q22.2-q22.3 |
| 207535_s_at | NFKB2 | 10q24 |
| 207536_s_at | TNFRSF9 | 1p36 |
| 207574_s_at | GADD45B | 19p13.3 |
| 207614_s_at | CUL1 | 7q36.1 |
| 207634_at | PDCD1 | 2q37.3 |
| 207641_at | TNFRSF13B | 17p11.2 |
| 207643_s_at | TNFRSF1A | 12p13.2 |
| 207679_at | PAX3 | 2q35 |
| 207680_x_at | PAX3 | 2q35 |
| 207686_s_at | CASP8 | 2q33-q34 |
| 207738_s_at | NCKAP1 | 2q32 |
| 207757_at | FLJ21628 | 5q35.3 |
| 207782_s_at | PSEN1 | 14q24.3 |
| 207816_at | LALBA | 12q13 |
| 207827_x_at | SNCA | 4q21 |
| 207829_s_at | BNIP1 | 5q33-q34 |
| 207841_at | SPIN2 | xp11.1 |
| 207849_at | IL2 | 4q26-q27 |
| 207892_at | TNFSF5 | xq26 |
| 207907_at | TNFSF14 | 19p13.3 |
| 207922_s_at | MAEA | 4p16.3 |
| 207925_at | CST5 | 20p11.21 |
| 207943_x_at | — | — |
| 207950_s_at | ANK3 | 10q21 |
| 207952_at | IL5 | 5q31.1 |
| 207953_at | — | — |
| 208000_at | GML | 8q24.3 |
| 208005_at | NTN1 | 17p13-p12 |
| 208014_x_at | AD7C-NTP | 1p36 |
| 208023_at | — | — |
| 208050_s_at | CASP2 | 7q34-q35 |
| 208060_at | PAX7 | 1p36.2-p36.12 |
| 208062_s_at | NRG2 | 5q23-q33 |
| 208169_s_at | PTGER3 | 1p31.2 |
| 208173_at | IFNB1 | 9p21 |
| 208200_at | — | — |
| 208289_s_at | EI24 | 11q24 |
| 208296_x_at | TNFAIP8 | 5q23.1 |
| 208309_s_at | MALT1 | 18q21 |
| 208315_x_at | TRAF3 | 14q32.32 |
| 208351_s_at | MAPK1 | 22q11.21 |
| 208352_x_at | ANK1 | 8p11.1 |
| 208353_x_at | ANK1 | 8p11.1 |
| 208368_s_at | BRCA2 | 13q12.3 |
| 208381_s_at | SGPL1 | 10q21 |
| 208402_at | IL17 | 6p12 |
| 208441_at | IGF1R | 15q26.3 |
| 208478_s_at | BAX | 19q13.3-q13.4 |
| 208485_x_at | CFLAR | 2q33-q34 |
| 208536_s_at | BCL2L11 | 2q13 |
| 208555_x_at | CST2 | 20p11.21 |
| 208588_at | FKSG2 | 8p11.2 |
| 208603_s_at | MAPK8IP2 | 22q13.33 |
| 208636_at | ACTN1 | 14q24.1-q24.2 |
| 208644_at | PARP1 | 1q41-q42 |
| 208652_at | PPP2CA | 5q23-q31 |
| 208791_at | CLU | 8p21-p12 |
| 208792_s_at | CLU | 8p21-p12 |
| 208822_s_at | DAP3 | 1q21-q22 |
| 208835_s_at | CROP | 17q21.33 |
| 208891_at | DUSP6 | 12q22-q23 |
| 208892_s_at | DUSP6 | 12q22-q23 |
| 208893_s_at | DUSP6 | 12q22-q23 |
| 208905_at | CYCS | 7p15.3 |
| 208920_at | SRI | 7q21.1 |
| 208921_s_at | SRI | 7q21.1 |
| 208945_s_at | BECN1 | 17q21 |
| 208946_s_at | BECN1 | 17q21 |
| 208977_x_at | TUBB2 | — |
| 209026_x_at | OK/SW-cl.56 | 6p21.33 |
| 209090_s_at | SH3GLB1 | 1p22 |
| 209091_s_at | SH3GLB1 | 1p22 |
| 209115_at | UBE1C | 3p24.3-p13 |
| 209124_at | MYD88 | 3p22 |
| 209165_at | AATF | 17q11.2-q12 |
| 209201_x_at | CXCR4 | 2q21 |
| 209230_s_at | P8 | 16p11.2 |
| 209239_at | NFKB1 | 4q24 |
| 209294_x_at | TNFRSF10B | 8p22-p21 |
| 209295_at | TNFRSF10B | 8p22-p21 |
| 209304_x_at | GADD45B | 19p13.3 |
| 209305_s_at | GADD45B | 19p13.3 |
| 209308_s_at | BNIP2 | 15q22.2 |
| 209310_s_at | CASP4 | 11q22.2-q22.3 |
| 209311_at | BCL2L2 | 14q11.2-q12 |
| 209318_x_at | PLAGL1 | 6q24-q25 |
| 209323_at | PRKRIR | 11q13.5 |
| 209339_at | SIAH2 | 3q25 |
| 209354_at | TNFRSF14 | 1p36.3-p36.2 |
| 209361_s_at | PCBP4 | 3p21 |
| 209364_at | BAD | 11q13.1 |
| 209372_x_at | TUBB; MGC8685 | 6p25 |
| 209406_at | BAG2 | 6p12.3-p11.2 |
| 209442_x_at | ANK3 | 10q21 |
| 209448_at | HTATIP2 | 11p15.1 |
| 209462_at | APLP1 | 19q13.1 |
| 209489_at | CUGBP1 | 11p11 |
| 209499_x_at | TNFSF13; TNFSF12-TNFSF13 | 17p13.1 |
| 209500_x_at | TNFSF13; TNFSF12-TNFSF13 | 17p13.1 |
| 209508_x_at | CFLAR | 2q33-q34 |
| 209539_at | ARHGEF6 | xq26 |
| 209544_at | RIPK2 | 8q21 |
| 209545_s_at | RIPK2 | 8q21 |
| 209615_s_at | PAK1 | 11q13-q14 |
| 209636_at | NFKB2 | 10q24 |
| 209686_at | S100B | 21q22.3 |
| 209719_x_at | SERPINB3 | 18q21.3 |
| 209720_s_at | SERPINB3 | 18q21.3 |
| 209788_s_at | ARTS-1 | 5q15 |
| 209790_s_at | CASP6 | 4q25 |
| 209799_at | PRKAA1 | 5p12 |
| 209802_at | — | — |
| 209803_s_at | PHLDA2 | 11p15.5 |
| 209811_at | CASP2 | 7q34-q35 |
| 209812_x_at | CASP2 | 7q34-q35 |
| 209831_x_at | DNASE2 | 19p13.2 |
| 209833_at | CRADD | 12q21.33-q23.1 |
| 209857_s_at | SPHK2 | 19q13.2 |
| 209863_s_at | TP73L | 3q27-q29 |
| 209875_s_at | SPP1 | 4q21-q25 |
| 209878_s_at | RELA | 11q13 |
| 209929_s_at | IKBKG | xq28 |
| 209939_x_at | CFLAR | 2q33-q34 |
| 209941_at | RIPK1 | 6p25.2 |
| 209969_s_at | STAT1 | 2q32.2 |
| 209970_x_at | CASP1 | 11q23 |
| 210017_at | MALT1 | 18q21 |
| 210018_x_at | MALT1 | 18q21 |
| 210025_s_at | CARD10 | 22q13.1 |
| 210026_s_at | CARD10 | 22q13.1 |
| 210042_s_at | CTSZ | 20q13 |
| 210074_at | CTSL2 | 9q22.2 |
| 210095_s_at | IGFBP3 | 7p13-p12 |
| 210101_x_at | SH3GLB1 | 1p22 |
| 210113_s_at | NALP1 | 17p13 |
| 210118_s_at | IL1A | 2q14 |
| 210140_at | CST7 | 20p11.21 |
| 210141_s_at | INHA | 2q33-q36 |
| 210164_at | GZMB | 14q11.2 |
| 210165_at | DNASE1 | 16p13.3 |
| 210168_at | C6 | 5p13 |
| 210252_s_at | MADD | 11p11.2 |
| 210253_at | HTATIP2 | 11p15.1 |
| 210260_s_at | TNFAIP8 | 5q23.1 |
| 210314_x_at | TNFSF13; TNFSF12-TNFSF13 | 17p13.1 |
| 210321_at | GZMH | 14q11.2 |
| 210324_at | C8G | 9q34.3 |
| 210334_x_at | BIRC5 | 17q25 |
| 210348_at | PNUTL2 | 17q22-q23 |
| 210367_s_at | PTGES | 9q34.3 |
| 210374_x_at | PTGER3 | 1p31.2 |
| 210375_at | PTGER3 | 1p31.2 |
| 210385_s_at | ARTS-1 | 5q15 |
| 210401_at | P2RX1 | 17p13.3 |
| 210405_x_at | TNFRSF10B | 8p22-p21 |
| 210474_s_at | CDC2L2; CDC2L1 | 1p36.3; 1p36 |
| 210476_s_at | PRLR | 5p14-p13 |
| 210483_at | MGC31957 | 8p21.2 |
| 210484_s_at | MGC31957; TNFRSF10C | 8p21.2; 8p22-p21 |
| 210511_s_at | INHBA | 7p15-p13 |
| 210512_s_at | VEGF | 6p12 |
| 210513_s_at | VEGF | 6p12 |
| 210538_s_at | BIRC3 | 11q22 |
| 210563_x_at | CFLAR | 2q33-q34 |
| 210564_x_at | CFLAR | 2q33-q34 |
| 210609_s_at | TP53I3 | 2p23.3 |
| 210639_s_at | APG5L | 6q21 |
| 210643_at | TNFSF11 | 13q14 |
| 210654_at | TNFRSF10D | 8p21 |
| 210655_s_at | FOXO3A | 6q21 |
| 210657_s_at | PNUTL2 | 17q22-q23 |
| 210685_s_at | UBE4B | 1p36.3 |
| 210708_x_at | CASP10 | 2q33-q34 |
| 210751_s_at | RGN | xp11.3 |
| 210756_s_at | NOTCH2 | 1p13-p11 |
| 210765_at | CSE1L | 20q13 |
| 210766_s_at | CSE1L | 20q13 |
| 210770_s_at | CACNA1A | 19p13.2-p13.1 |
| 210775_x_at | CASP9 | 1p36.3-p36.1 |
| 210792_x_at | SIVA | 14q32.33 |
| 210831_s_at | PTGER3 | 1p31.2 |
| 210832_x_at | PTGER3 | 1p31.2 |
| 210833_at | — | — |
| 210834_s_at | PTGER3 | 1p31.2 |
| 210847_x_at | TNFRSF25 | 1p36.2 |
| 210865_at | TNFSF6 | 1q23 |
| 210907_s_at | PDCD10 | 3q26.1 |
| 210955_at | CASP10 | 2q33-q34 |
| 210968_s_at | RTN4 | 2p16.3 |
| 210975_x_at | FASTK | 7q35 |
| 211078_s_at | STK3 | 8q22.2 |
| 211085_s_at | STK4 | 20q11.2-q13.2 |
| 211127_x_at | EDA | xq12-q13.1 |
| 211128_at | EDA | xq12-q13.1 |
| 211129_x_at | EDA | xq12-q13.1 |
| 211130_x_at | EDA | xq12-q13.1 |
| 211131_s_at | EDA | xq12-q13.1 |
| 211140_s_at | CASP2 | 7q34-q35 |
| 211152_s_at | PRSS25 | 2p12 |
| 211153_s_at | TNFSF11 | 13q14 |
| 211163_s_at | TNFRSF10C | 8p22-p21 |
| 211193_at | TP73L | 3q27-q29 |
| 211194_s_at | TP73L | 3q27-q29 |
| 211195_s_at | TP73L | 3q27-q29 |
| 211214_s_at | DAPK1 | 9q34.1 |
| 211255_x_at | — | — |
| 211265_at | PTGER3 | 1p31.2 |
| 211269_s_at | IL2RA | 10p15-p14 |
| 211277_x_at | APP | 21q21.3 |
| 211282_x_at | TNFRSF25 | 1p36.2 |
| 211289_x_at | CDC2L2; CDC2L1 | 1p36.3; 1p36 |
| 211298_s_at | ALB | 4q11-q13 |
| 211300_s_at | TP53 | 17p13.1 |
| 211316_x_at | CFLAR | 2q33-q34 |
| 211317_s_at | CFLAR | 2q33-q34 |
| 211333_s_at | TNFSF6 | 1q23 |
| 211338_at | IFNA2 | 9p22 |
| 211366_x_at | CASP1 | 11q23 |
| 211367_s_at | CASP1 | 11q23 |
| 211368_s_at | CASP1 | 11q23 |
| 211373_s_at | PSEN2 | 1q31-q42 |
| 211464_x_at | CASP6 | 4q25 |
| 211475_s_at | BAG1 | 9p12 |
| 211489_at | ADRA1A | 8p21-p11.2 |
| 211490_at | ADRA1A | 8p21-p11.2 |
| 211491_at | ADRA1A | 8p21-p11.2 |
| 211492_s_at | ADRA1A | 8p21-p11.2 |
| 211495_x_at | TNFSF13; TNFSF12-TNFSF13 | 17p13.1 |
| 211509_s_at | RTN4 | 2p16.3 |
| 211524_at | NFKB2 | 10q24 |
| 211526_s_at | RTEL1 | 20q13.3 |
| 211527_x_at | VEGF | 6p12 |
| 211546_x_at | SNCA | 4q21 |
| 211553_x_at | APAF1 | 12q23 |
| 211554_s_at | APAF1 | 12q23 |
| 211704_s_at | SPIN2 | xp11.1 |
| 211706_s_at | CDK11 | 6q21 |
| 211714_x_at | OK/SW-cl.56 | 6p21.33 |
| 211725_s_at | BID | 22q11.1 |
| 211786_at | TNFRSF9 | 1p36 |
| 211822_s_at | NALP1 | 17p13 |
| 211824_x_at | NALP1 | 17p13 |
| 211832_s_at | MDM2 | 12q14.3-q15 |
| 211833_s_at | BAX | 19q13.3-q13.4 |
| 211834_s_at | TP73L | 3q27-q29 |
| 211841_s_at | TNFRSF25 | 1p36.2 |
| 211851_x_at | BRCA1 | 17q21 |
| 211856_x_at | CD28 | 2q33 |
| 211861_x_at | CD28 | 2q33 |
| 211862_x_at | CFLAR | 2q33-q34 |
| 211888_x_at | CASP10 | 2q33-q34 |
| 211899_s_at | TRAF4 | 17q11-q12 |
| 211909_x_at | PTGER3 | 1p31.2 |
| 211910_at | — | — |
| 211917_s_at | PRLR | 5p14-p13 |
| 211919_s_at | CXCR4 | 2q21 |
| 211943_x_at | TPT1 | 13q12-q14 |
| 212038_s_at | VDAC1 | 5q31 |
| 212048_s_at | YARS | 1p35.1 |
| 212099_at | RHOB | 2p24 |
| 212143_s_at | IGFBP3 | 7p13-p12 |
| 212171_x_at | VEGF | 6p12 |
| 212213_x_at | OPA1 | 3q28-q29 |
| 212214_at | OPA1 | 3q28-q29 |
| 212271_at | MAPK1 | 22q11.21 |
| 212312_at | BCL2L1 | 20q11.21 |
| 212320_at | OK/SW-cl.56 | 6p21.33 |
| 212322_at | SGPL1 | 10q21 |
| 212344_at | SULF1 | 8q13.2-q13.3 |
| 212353_at | SULF1 | 8q13.2-q13.3 |
| 212354_at | SULF1 | 8q13.2-q13.3 |
| 212355_at | KIAA0323 | 14q11.2 |
| 212356_at | KIAA0323 | 14q11.2 |
| 212367_at | FEM1B | 15q22 |
| 212373_at | FEM1B | 15q22 |
| 212374_at | FEM1B | 15q22 |
| 212377_s_at | NOTCH2 | 1p13-p11 |
| 212401_s_at | CDC2L2 | 1p36.3 |
| 212422_at | PDCD11 | 10q24.33 |
| 212424_at | PDCD11 | 10q24.33 |
| 212508_at | MOAP1 | 14q32 |
| 212562_s_at | — | — |
| 212593_s_at | PDCD4 | 10q24 |
| 212594_at | PDCD4 | 10q24 |
| 212664_at | TUBB5 | 19p13.3 |
| 212687_at | LIMS1 | 2q12.3-q13 |
| 212722_s_at | PTDSR | 17q25 |
| 212723_at | PTDSR | 17q25 |
| 212849_at | AXIN1 | 16p13.3 |
| 212869_x_at | TPT1 | 13q12-q14 |
| 212897_at | CDK11 | 6q21 |
| 212899_at | CDK11 | 6q21 |
| 213026_at | APG12L | 5q21-q22 |
| 213093_at | PRKCA | 17q22-q23.2 |
| 213100_at | UNC5B | 10q22.1 |
| 213213_at | DATF1 | 20q13.33 |
| 213220_at | LOC92482 | 10q25.2 |
| 213224_s_at | LOC92482 | 10q25.2 |
| 213254_at | KIAA1093 | 22q13.1 |
| 213274_s_at | CTSB | 8p22 |
| 213275_x_at | FDFT1 | 8p23.1-p22 |
| 213281_at | — | — |
| 213338_at | RIS1 | 3p21.3 |
| 213373_s_at | CASP8 | 2q33-q34 |
| 213405_at | RAB22A | 20q13.32 |
| 213443_at | — | — |
| 213468_at | ERCC2 | 19q13.3 |
| 213530_at | RAB3GAP | 2q21.3 |
| 213531_s_at | RAB3GAP | 2q21.3 |
| 213532_at | ADAM17 | 2p25 |
| 213538_at | SON | 21q22.11 |
| 213560_at | — | — |
| 213579_s_at | EP300 | 22q13.2 |
| 213581_at | PDCD2 | 6q27 |
| 213585_s_at | PDCD2 | 6q27 |
| 213596_at | CASP4 | 11q22.2-q22.3 |
| 213726_x_at | TUBB2 | — |
| 213763_at | HIPK2 | 7q32-q34 |
| 213786_at | TAX1BP1 | 7p15 |
| 213790_at | ADAM12 | 10q26.3 |
| 213829_x_at | TNFRSF6B | 20q13.3 |
| 213895_at | EMP1 | 12p12.3 |
| 213921_at | — | — |
| 213933_at | PTGER3 | 1p31.2 |
| 213972_at | — | — |
| 213975_s_at | LYZ; LILRB1 | 12q15; 19q13.4 |
| 214012_at | ARTS-1 | 5q15 |
| 214033_at | — | — |
| 214034_at | ARTS-1 | 5q15 |
| 214040_s_at | GSN | 9q33 |
| 214056_at | — | — |
| 214057_at | — | — |
| 214090_at | — | — |
| 214105_at | — | — |
| 214114_x_at | FASTK | 7q35 |
| 214203_s_at | PRODH | 22q11.21 |
| 214228_x_at | TNFRSF4 | 1p36 |
| 214237_x_at | PAWR | 12q21 |
| 214306_at | OPA1 | 3q28-q29 |
| 214329_x_at | — | — |
| 214450_at | CTSW | 11q13.1 |
| 214467_at | GPR65 | 14q31-q32.1 |
| 214486_x_at | CFLAR | 2q33-q34 |
| 214491_at | SSTR3 | 22q13.1 |
| 214499_s_at | BCLAF1 | 6q22-q23 |
| 214575_s_at | AZU1 | 19p13.3 |
| 214578_s_at | ROCK1 | 18q11.1 |
| 214581_x_at | TNFRSF21 | 6p21.1-12.2 |
| 214617_at | PRF1 | 10q22 |
| 214618_at | CFLAR | 2q33-q34 |
| 214629_x_at | RTN4 | 2p16.3 |
| 214641_at | COL4A3 | 2q36-q37 |
| 214727_at | BRCA2 | 13q12.3 |
| 214786_at | MAP3K1 | 5q11.2 |
| 214793_at | DUSP7 | 3p21 |
| 214837_at | ALB | 4q11-q13 |
| 214917_at | PRKAA1 | 5p12 |
| 214933_at | CACNA1A | 19p13.2-p13.1 |
| 214953_s_at | APP | 21q21.3 |
| 214959_s_at | API5 | 11p12-q12 |
| 214960_at | API5 | 11p12-q12 |
| 214988_s_at | SON | 21q22.11 |
| 214992_s_at | DNASE2 | 19p13.2 |
| 215028_at | SEMA6A | 5q23.1 |
| 215037_s_at | BCL2L1 | 20q11.21 |
| 215096_s_at | ESD | 13q14.1-q14.2 |
| 215158_s_at | DEDD | 1q23.3 |
| 215184_at | DAPK2 | 15q22.31 |
| 215195_at | PRKCA | 17q22-q23.2 |
| 215223_s_at | SOD2 | 6q25.3 |
| 215233_at | PTDSR | 17q25 |
| 215329_s_at | CDC2L2; CDC2L1 | 1p36.3; 1p36 |
| 215346_at | TNFRSF5 | 20q12-q13.2 |
| 215440_s_at | BEXL1 | Xq22.1-q22.3 |
| 215479_at | SEMA6A | 5q23.1 |
| 215494_at | — | — |
| 215533_s_at | UBE4B | 1p36.3 |
| 215539_at | BIRC6 | 2p22-p21 |
| 215545_at | ERCC3 | 2q21 |
| 215628_x_at | PPP2CA | 5q23-q31 |
| 215719_x_at | TNFRSF6 | 10q24.1 |
| 215744_at | FUS | 16p11.2 |
| 215851_at | EVI1 | 3q24-q28 |
| 215913_s_at | GULP1 | 2q32.3-q33 |
| 215915_at | — | — |
| 215976_at | DBC1 | 9q32-q33 |
| 216015_s_at | CIAS1 | 1q44 |
| 216016_at | CIAS1 | 1q44 |
| 216038_x_at | DAXX | 6p21.3 |
| 216042_at | TNFRSF25 | 1p36.2 |
| 216059_at | PAX3 | 2q35 |
| 216220_s_at | ADORA1 | 1q32.1 |
| 216226_at | TAF4B | 18q11.2 |
| 216252_x_at | TNFRSF6 | 10q24.1 |
| 216253_s_at | PARVB | 22q13.2-q13.33 |
| 216254_at | PARVB | 22q13.2-q13.33 |
| 216325_x_at | RTEL1 | 20q13.3 |
| 216326_s_at | HDAC3 | 5q31 |
| 216347_s_at | PPP1R13B | 14q32.33 |
| 216396_s_at | EI24 | 11q24 |
| 216589_at | — | — |
| 216598_s_at | CCL2 | 17q11.2-q21.1 |
| 216638_s_at | PRLR | 5p14-p13 |
| 216761_at | PDCD8 | Xq25-q26 |
| 216766_at | PRKCE | 2p21 |
| 216776_at | BCAP29 | 7q22-q31 |
| 216876_s_at | IL17 | 6p12 |
| 216893_s_at | COL4A3 | 2q36-q37 |
| 216898_s_at | COL4A3 | 2q36-q37 |
| 216995_x_at | MKRN2 | 3p25 |
| 217028_at | CXCR4 | 2q21 |
| 217029_at | BAX | 19q13.3-q13.4 |
| 217140_s_at | VDAC1 | 5q31 |
| 217373_x_at | MDM2 | 12q14.3-q15 |
| 217379_at | — | — |
| 217399_s_at | FOXO3A | 6q21 |
| 217465_at | — | — |
| 217500_at | — | — |
| 217559_at | RPL10L | 14q13-q21 |
| 217604_at | — | — |
| 217607_x_at | EIF4G2 | 11p15 |
| 217631_at | IDI2; GTPBP4 | 10p15.3; 10p15-p14 |
| 217657_at | — | — |
| 217676_at | — | — |
| 217744_s_at | PERP | 6q24 |
| 217746_s_at | PDCD6IP | 3p23 |
| 217789_at | SNX6 | 14q13.1 |
| 217840_at | DDX41 | 5q35.3 |
| 217911_s_at | BAG3 | 10q25.2-q26.2 |
| 217923_at | PEF | 1p34 |
| 217955_at | BCL2L13 | 22q11 |
| 217963_s_at | NGFRAP1 | xq22.2 |
| 217996_at | PHLDA1 | 12q15 |
| 217997_at | PHLDA1 | 12q15 |
| 217998_at | PHLDA1 | 12q15 |
| 217999_s_at | — | — |
| 218000_s_at | PHLDA1 | 12q15 |
| 218024_at | BRP44L | 6q27 |
| 218056_at | BFAR | 16p13.12 |
| 218080_x_at | FAF1 | 1p33 |
| 218085_at | SNF7DC2 | 9p13.3 |
| 218088_s_at | RRAGC | 1p34 |
| 218145_at | TRIB3 | 20p13-p12.2 |
| 218182_s_at | CLDN1 | 3q28-q29 |
| 218224_at | PNMA1 | 14q24.3 |
| 218229_s_at | POGK | 1q24.1 |
| 218286_s_at | RNF7 | 3q22-q24 |
| 218297_at | C10orf97 | 10p13 |
| 218325_s_at | DATF1 | 20q13.33 |
| 218368_s_at | TNFRSF12A | 16p13.3 |
| 218373_at | FTS | 16q12.2 |
| 218380_at | NALP1 | 17p13 |
| 218398_at | MRPS30 | 5q11 |
| 218573_at | MAGEH1 | xp11.22 |
| 218609_s_at | NUDT2 | 9p13 |
| 218651_s_at | FLJ11196 | 15q23 |
| 218732_at | Bit1 | 17q23.2 |
| 218833_at | ZAK | 2q24.2 |
| 218845_at | DUSP22 | 6p25.3 |
| 218849_s_at | RAI | 19q13.32 |
| 218856_at | TNFRSF21 | 6p21.1-12.2 |
| 218878_s_at | SIRT1 | 10q21.3 |
| 218880_at | FOSL2 | 2p23.3 |
| 218881_s_at | FOSL2 | 2p23.3 |
| 218996_at | TFPT | 19q13 |
| 219019_at | LRDD | 11p15.5 |
| 219028_at | HIPK2 | 7q32-q34 |
| 219111_s_at | DDX54 | 12q24.13 |
| 219232_s_at | EGLN3 | 14q13.1 |
| 219275_at | PDCD5 | 19q12-q13.1 |
| 219329_s_at | C2orf28 | 2p23.3 |
| 219350_s_at | DIABLO | 12q24.31 |
| 219356_s_at | SNF7DC2 | 9p13.3 |
| 219366_at | AVEN | 15q13.1 |
| 219398_at | CIDEC | 3p25.3 |
| 219411_at | ELMO3 | 16q22.1 |
| 219422_at | — | — |
| 219423_x_at | TNFRSF25 | 1p36.2 |
| 219500_at | CLC | 11q13.3 |
| 219551_at | EAF2 | 3q13.33 |
| 219566_at | PLEKHF1 | 19q12 |
| 219618_at | IRAK4 | 12q12 |
| 219624_at | BAG4 | 8p12 |
| 219765_at | FLJ12586 | 19q13.43 |
| 219786_at | MTL5 | 11q13.2-q13.3 |
| 219817_at | — | — |
| 219875_s_at | PNAS-4 | 1q44 |
| 219933_at | GLRX2 | 1q31.2-q31.3 |
| 220034_at | IRAK3 | 12q14.3 |
| 220044_x_at | CROP | 17q21.33 |
| 220048_at | EDAR | 2q11-q13 |
| 220049_s_at | PDCD1LG2 | 9p24.2 |
| 220066_at | CARD15 | 16p12-q21 |
| 220162_s_at | CARD9 | 9q34.3 |
| 220187_at | TNFAIP9 | 7q21.12 |
| 220212_s_at | THADA | 2p21 |
| 220363_s_at | ELMO2 | 20q13 |
| 220402_at | P53AIP1 | 11q24 |

TABLE 1

| | | | Clinical | | Chemotherapy | Response | |
|---|---|---|---|---|---|---|---|
| Tumour | Age | Tumour Site | stage | Histopathology | (cycles) | (RECIST) | Clinical presentation |
| LT1 | 55 | Right upper lobe | T2N2M0 | Squamous cell | MVP (3) | Stable disease | Male, WHO PS 0, |
| | | | | carcinoma; | | | 2 stone weight loss, |
| | | | | moderately | | | cough, smoker |
| | | | | differentiated | | | |
| LT2 | 72 | Right lower lobe | T3N1M0 | Squamous cell | MVP (3) | Partial response | Female, WHO PS 1, |
| | | | | carcinoma; | | | fatigue, night sweats, |
| | | | | poorly | | | 2 stone weight loss, |
| | | | | differentiated | | | smoker |
| LT3 | 45 | Left upper lobe | T2N0M0 | Adenocarcinoma | MVP (3) | Partial response | Female, WHO PS 0, |
| | | | | | | | Cough, Dyspnoea, smoker |
| LT4 | 68 | Right upper lobe | T2N1M0 | Adenocarcinoma; | MVP (3) | Partial response | Female, WHO PS 1, |
| | | | | poorly | | | Cough, haemoptysis, |
| | | | | differentiated | | | fatigue, smoker |
| LT5 | 55 | Right upper lobe | T2N1M0 | Adenocarcinoma; | MVP (3) | Stable disease | Female, WHO PS 1, |
| | | | | poorly | | | dyspnoea, cough, |
| | | | | differentiated | | | lobar collapse, smoker |
| LT6 | 73 | Left hilum | T2N1M0 | Squamous cell | MVP (3) | Partial response | Male, WHO PS 1, |
| | | | | carcinoma; | | | persistent cough, |
| | | | | poorly | | | Smoker |
| | | | | differentiated | | | |
| LT7 | 60 | Right upper lobe | T2N0M0 | Peri parenchymal | NP (3) | Stable disease | Female, WHO PS 1, |
| | | | | adenocarcinoma; | | | Cough, weight loss 1 stone, |
| | | | | well | | | smoker |
| | | | | differentiated | | | |
| LT8 | 50 | Right lower lobe | T2N0M0 | Peripheral | NP (3) | Stable disease | Female WHO PS 1, |
| | | | | adenocarcinoma | | | cough, smoker |
| | | | | predominately | | | |
| | | | | mucinous | | | |
| | | | | bronchioalveolar | | | |
| | | | | type | | | |

TABLE 2

| | | | | | | Fold Change | | | |
|---|---|---|---|---|---|---|---|---|---|
| | | | | | | Non-responding to Responding² | | | |
| | | | | | | | Test set⁴ | | |
| | Gene | | | | | Training | (n = 8) | | Complete |
| Gene | Name | | Chromosomal | | Predictive | set³ | Full | Prechem⁵ | set⁶ |
| Title | (HUGO ID) | Probe set id | location | Function | strength¹ | (n = 8) | (n = 8) | (n = 6) | (n = 16) |
| Serpin B3 | SERPINB3 | 209720_s_at | 18q21.3 | Protease Inhibitor/Cell | 4.25 | +50.54 | +4.97 | +3.40 | +7.64 |
| | | | | death | | | | | |
| Hypothetical | FLJ23049 | 220269_at | 3q26.1 | Unknown | 2.64 | +9.87 | +2.16 | +3.56 | +3.41 |
| Protein FLJ 23049 | | | | | | | | | |
| Hydroxysteroid | HSD17B2 | 204818_at | 16q24.1-24.2 | Steroid Biosynthesis | 2.64 | +7.21 | +2.10 | +2.36 | +1.91 |
| (17B) | | | | | | | | | |
| dehydrogenase 2 | | | | | | | | | |
| Cell Division | CDC20 | 202870_s_at | 1p34.1 | Cell cycle control/ | 4.25 | −6.16 | −4.07 | −4.42 | −4.32 |
| Cycle homolog 20 | | | | Anaphase promoting | | | | | |
| | | | | complex formation | | | | | |
| Fibronectin type 3 | FNDC3 | 215910_s_at | 13q14.2 | Unknown | 2.64 | +6.12 | +2.69 | +2.43 | +3.69 |
| domain containing 3 | | | | | | | | | |
| Hypothetical Protein | FLJ11767 | 220156_at | | Unknown | 2.64 | +6.07 | +2.5 | +2.26 | +2.68 |
| FLJ11767 | | | | | | | | | |
| Semaphorin 3D | SEMA3D | 215643_at | 7q21.11 | Secreted protein | 4.25 | −5.85 | −2.45 | −3.68 | −2.54 |
| | | | | unknown function | | | | | |
| Cystatin SN | CST1 | 206224_at | 20p11.21 | Protease Inhibitor | 2.64 | +5.78 | +4.83 | +3.54 | +3.94 |
| Sperm Associated | SPAG6 | 210032_s_at | 10p12.2 | Spermatid motility/ | 4.25 | +5.49 | +2.76 | +2.31 | +2.40 |
| Antigen 6 | | | | microtubule stability | | | | | |
| Potassium inwardly | KCNJ16 | 219564_at | 17q23.1-24.2 | Potassium channel at | 4.25 | +5.15 | +2.20 | +2.10 | +2.72 |
| rectifying channel | | | | cell surface | | | | | |
| subfamily J | | | | | | | | | |
| member 16 | | | | | | | | | |
| Histone 1 H2bg | HIST1H2BG | 215779_s_at | 6p21.3 | Nucleosome structure | 2.64 | −4.93 | −2.61 | −2.47 | −2.88 |
| Soluble Adenyl | SAC | 214547_at | 1q24 | Intracellular signalling; | 4.25 | −4.87 | −2.06 | −2.32 | −3.09 |
| Cyslase | | | | purine metabolism; | | | | | |
| | | | | cAMP biosynthesis | | | | | |
| Gelsolin | GSN | 214040_s_at | 9q33 | Cell death/senescence | 4.25 | +4.85 | +2.57 | +3.83 | +2.05 |
| Cellular Retinoic | CRABP1 | 205350_at | 15q22 | Retinoic acid biology | 4.25 | −4.31 | −2.13 | −2.50 | −2.81 |
| Binding Protein 1 | | | | | | | | | |
| Carbonic Anhydrase | CA12 | 210735_s_at* | 15q24 | Bicarbonate/pH | 4.25 | +4.21 | +3.17 | +3.79 | +2.34 |
| isoform 12 | | 215867_x_at | | balance; Nitrogen | | | | | |
| | | 214164_x_at | | metabolism | | | | | |
| Zinc Finger | ZNF444 | 218707_at | 19q13.43 | Endothelial Cell | 4.25 | −4.18 | −2.29 | −2.12 | −2.70 |
| protein 444 | | | | transcription factor | | | | | |
| v-myb | MYB | 204798_at | 6q22-23 | Known oncogene, | 4.25 | −4.14 | −2.03 | −2.94 | −2.18 |
| myeloblastosis viral | | | | transcription factor. | | | | | |
| oncogene homolog | | | | | | | | | |

TABLE 3

| | | Clinical | | | | | | | |
|---|---|---|---|---|---|---|---|---|---|
| | | Stage | | Relapse | Disease Free | | | | Response |
| Tumour | Age | (resection) | Histolopathology | Date | Survival | Site or Relapse | Chemotherapy | Cycles | (RECIST) |
| LT9 | 67 | T2N0M0 | Adenocarcinomas | No | N/A | N/A | Neoadjuvant NP | 3 | Partial |
| | | | | | | | | | response |
| LT10 | 60 | T3N2M0 | Adenocarcinomas | No | N/A | N/A | Neoadjuvant | 3 | Partial |
| | | | | | | | Cisplatin and | | response |
| | | | | | | | Paclitaxel | | |
| LT11 | 69 | T2N2M0 | Adenocarcinomas | Yes | 20 months | lung, | Gemcitabine and | 4 | Partial |
| | | | | | | mediastinum | Cisplatin | | response |
| LT12 | 68 | T2N2M0 | Adenocarcinoma | Yes | 3 months | Lung | Carboplatin and | 4 | Complete |
| | | | | | | | Paclitaxel | | response |
| LT13 | 61 | T3N2M0 | Squamous cell | Yes | 25 months | Lung | MVP | 3 | Stable disease |
| | | | carcinoma | | | | | | |
| LT14 | 64 | T2N0M0 | Adenocarcinoma | Yes | 37 months | Lymph Nodes | MVP | 4 | Stable disease |
| LT15 | 63 | T2N1M0 | Adenocarcinoma | Yes | 24 months | mediastinum, | Carboplatin and | 1 | Progressive |
| | | | | | | adrenal, bone | Paclitaxel | | disease |
| LT16 | 67 | T2NOM0 | Adenocarcinoma | Yes | 19 months | lung | Carboplatin and | 2 | Progressive |
| | | | | | | | Cocetaxel | | disease |

TABLE 4
A) Non-Responders

| | | | | Fold |
|---|---|---|---|---|
| | | | Non- | Change |
| | Gene Name | | responder | Tumour² |
| Gene Title | HUGO ID | Probe set ID | T:N¹ | NR:R |
| Serpin B3 | SERPINB3 | 209720_s_at | 7.98 | 50.17 |
| Collagen Type IV | COL4A3 | 214641_at | −1.92 | −2.19 |
| alpha 3 chain | | | | |
| Angiotensin II | AGRT2 | 222321_a | −3.18 | −12.27 |
| receptor type 2 | | 207294_at* | | |

B) Responders

| | | | | Fold |
|---|---|---|---|---|
| | | | | Change |
| | Gene Name | | Responder | Tumour² |
| Gene Title | HUGO ID | Probe set ID | T:N¹ | R:NR |
| Cystatin C | CST3 | 201360_at | −1.64 | −2.87 |
| Survivin | BIRC5 | 202095_s_at | 1.99 | 2.96 |
| Dual specitificity | DUSP6 | 208892_s_at | −1.71 | −1.66 |
| phosphatase 6 | | | | |
| TNF superfamily | TNFRSF21 | 218856_at | 1.59 | 1.53 |
| receptor | | | | |
| member 21 | | | | |
| STAT1 | STAT1 | 209969_s_at | 1.64 | 1.73 |
| Epithelial | EMP3 | 203729_at | −1.59 | −1.90 |
| Membrane | | | | |
| Protein 3 | | | | |
| Tissue Inhibitor | TIMP3 | 201150_s_at | −1.82 | −1.57 |
| of | | | | |
| Metalloprotease-3 | | | | |
| Nucleophosmin | NPMI | 221923_at | 1.62 | 1.62 |

| | | | | |
|---|---|---|---|---|
| | All | Non- | | p-value |
| Variable | patients | responders | Responders | (NR vs R) |

(a)
Pre-Chemotherapy Tissues (n = 36)

| | | | | |
|---|---|---|---|---|
| Age (< or > 70 y) | | | | 1.00 |
| Mean | 62.8 | 62.3 | 63.4 | |
| Range | 44-78 | 44-78 | 46-74 | |
| Sex | | | | 1.00 |
| Male | 29 | 15 | 14 | |
| Female | 7 | 4 | 3 | |
| Smoking | | | | 0.684 |
| Smoker | 29 | 16 | 13 | |
| Non-smoker | 7 | 3 | 4 | |
| Weight loss | | | | 0.605 |
| <10% | 32 | 16 | 16 | |
| >10% | 4 | 3 | 1 | |
| Histology-Type | | | | 0.504 |
| Adenocarcinoma | 18 | 11 | 7 | |
| Squamous | 18 | 8 | 10 | |
| Histology-grade | | | | 1.00 |
| (poor vs mod/well) | | | | |
| Poor | 10 | 5 | 5 | |
| Moderate | 21 | 12 | 9 | |
| Well | 5 | 2 | 3 | |
| WHO PS | | | | 0.344 |
| 0 | 11 | 4 | 7 | |
| 1 | 25 | 15 | 10 | |
| Stage | | | | 0.344 |
| (Early vs. Advanced) | | | | |
| IA and IB | 3 | 1 | 2 | |
| II A and B | 3 | 1 | 2 | |
| IIIA | 7 | 3 | 4 | |
| IIIB | 15 | 6 | 9 | |
| IV | 8 | 8 | 0 | |
| Chemotherapy | | | | 0.542 |
| DC | 14 | 8 | 6 | |
| MVP | 21 | 11 | 10 | |
| NP | 1 | 0 | 1 | |

(b)
Post-Chemotherapy Tissues (n = 13)

| | | | | |
|---|---|---|---|---|
| Age (< or >70 y) | | | | 0.462 |
| Mean | 62.8 | 60.3 | 65.8 | |
| Range | 42-73 | 42-72 | 53-73 | |
| Sex | | | | 1.00 |
| Male | 6 | 3 | 3 | |
| Female | 7 | 4 | 3 | |
| Smoking | | | | 1.00 |
| Smoker | 11 | 6 | 5 | |
| Non-smoker | 2 | 1 | 1 | |
| Weight loss | | | | 1.00 |
| <10% | 13 | 7 | 6 | |
| >10% | 0 | 0 | 0 | |
| Histology-Type | | | | 1.00 |
| Adenocarcinoma | 7 | 4 | 3 | |
| Squamous | 6 | 3 | 3 | |
| Histology-Grade | | | | 1.00 |
| (Poor vs. mod/well) | | | | |
| Poor | 1 | 1 | 0 | |
| Moderate | 8 | 4 | 4 | |
| Well | 4 | 2 | 2 | |
| WHO PS | | | | 0.061 |
| 0 | 9 | 7 | 2 | |
| 1 | 4 | 0 | 4 | |
| Stage | | | | 1.00 |
| (Early vs. Advanced) | | | | |
| IA and IB | 4 | 2 | 2 | |
| II A and B | 4 | 2 | 2 | |
| IIIA | 2 | 1 | 1 | |
| IIIB | 0 | 0 | 0 | |
| IV | 3 | 2 | 1 | |
| Chemotherapy | | | | 0.462 |
| MVP | 12 | 7 | 5 | |
| NP | 1 | 0 | 1 | |

TABLE 6

| Marker | |
|---|---|
| | Serpin B3 + |
| | (Cystatin C/ |
| Serpin B3 | Cathepsin B) |

| Response | pre | post | pre | post |
|---|---|---|---|---|
| Non- | 0.79 | 2.14 | 2.22 | 3.21 |
| responder | (n = 19) | (n = 7) | (n = 19) | (n = 7) |
| Responder | 0.29 | 0.67 | 1.31 | 1.59 |
| | (n = 17) | (n = 6) | (n = 17) | (n = 6) |
| p-value | 0.045 | 0.01 | 0.007 | 0.021 |

TABLE 7

| | | | p-value |
|---|---|---|---|
| | SCC (n = 176) | AC (n = 75) | (SCC v AC) |
| Age | | | 0.983 |
| Mean | 63.2 (27-81) | 61.6 (42-78) | |
| >70 | 150 (85%) | 64 (85%) | |
| <70 | 26 (15%) | 11 (15%) | |
| Sex | | | 0.296 |
| Male | 105 (60%) | 49 (65%) | |
| Female | 71 (40%) | 26 (35%) | |
| Stage | | | 0.755 |
| IA/B | 78 (43%) | 31 (41%) | |
| IIA/B | 64 (36%) | 32 (42%) | |
| IIIA | 34 (19%) | 12 (17%) | |
| Grade | | | 0.880 |
| Well | 17 (7%) | 7 (9%) | |
| Moderate | 65 (38%) | 30 (40%) | |
| Poor | 94 (55%) | 38 (51%) | |

TABLE 8

| | Tumour | | |
|---|---|---|---|
| Serpin B3 | SCC | ACs | |
| IHC Score | (n = 176) | (n = 75) | p-value |
| Negative (0) | 62 (35%) | 45 (60%) | P < 0.0001 |
| Positive (1, 2 or 3) | 114 (65%) | 30 (40%) | |

1. A method of predicting whether or not a cancer patient is suitable for chemotherapy, such as platinum based chemotherapy comprising the steps of:
a) providing a sample of non-small cell lung cancer (NSCLC) tumour tissue; and
b) detecting Serpin B3 expression in said tumour tissue, wherein if a proportion of tumour cells from the sample of tumour tissue are expressing Serpin B3, it is predicted that the patient is a poor candidate for response to chemotherapy and is not therefore suitable for chemotherapy.

2. The method of predicting whether or not a cancer patient is suitable for chemotherapy, according to claim 1, further comprising the step of:
c) detecting cystatin C and cathepsin B expression in said tumour tissue, wherein if a proportion of tumour cells from the sample of tumour tissue are expressing Serpin B3, and cystatin C relative to cathepsin B, it is predicted that the patient is a poor candidate for response to chemotherapy and is not therefore suitable for chemotherapy.

3. The method according to claim 2 wherein a patient is suitable for chemotherapy when they display a negative test for non-response (immunohistochemical value ≦2).

4. The method according to claim 2 wherein an immunohistochemical value of greater than 2 (positive test for non-response) indicates that the cancer patient is not suitable for platinum based therapy.

5. The method according to claim 1 wherein capthepsins L, K and/or S and/or papain is/are also detected in addition to Serpin B3 in order to allow prediction of whether or not a cancer patient is suitable for chemotherapy.

6. The method of predicting whether or not a cancer patient is suitable for chemotherapy, according to claim 1 further comprising the steps of:
c) detecting a level of expression of Serpin B3 and one or more of the following 16 genes:
Hypothetical Protein FLJ23049 (FLJ23049), Hydroxysteroid (17β) dehydrogenase 2 (HSD17B2), Cell Division Cycle homolog 20 (CDC20), Fibronectin type 3 domain containing 3A (FNDC3A), EF-hand calcium binding domain 1 (EFCAB1; previous name FLJ11767), Semaphorin 3D (SEMA3D), Cystatin SN (CST1), Sperm Associated Antigen 6 (SPAG6), Potassium inwardly rectifying channel subfamily J member 16 (KCNJ16), Histone 1 H2bg (H1 ST1H2BG), testicular Soluble Adenylyl Cyclase (SAC), Gelsolin (GSN), Cellular Retinoic Binding Protein 1 (CRABP1), Carbonic Anhydrase isoform 12 (CA12), Zinc Finger protein 444 (ZNF444), and v-myb myeloblastosis viral oncogene homolog (MYB); and
d) predicting whether or not the patient is likely to be a responder or non-responder to a chemotherapy based on a profile of expression of said genes and therefore suitable or otherwise for chemotherapy treatment.

7. The method according to claim 6 wherein a level of at least 5, of said genes is detected in order to provide a profile of expression.

8. The method of predicting whether or not a cancer patient is suitable for chemotherapy, according to claim 1 further comprising the step of:
a) detecting a level of expression of one or more of the following 10 genes:
Collagen Type IV alpha 3 chain (COL4A3), Angiotensin II receptor type 2 (AGRT2), Cystatin C (CST3), Survivin (BIRC5), Dual specificity phosphatase 6 (DUSP6), TNF superfamily receptor member 21 (TNFRS21), STAT1 (STAT1), Epithelial Membrane Protein 3 (EMP3), Tissue Inhibitor of Metalloprotease-3 (TIMP3) and Nucleophosmin (NPM1); and
d) predicting whether or not the patient is likely to be a responder or non-responder to chemotherapy based on a profile of expression of said genes and therefore suitable or otherwise for platinum based chemotherapy treatment.

9. The method according to claim 8 wherein a level of at least 4, of said genes is detected in order to provide a profile of expression.

10. The method according to claim 6 further comprising the detection of one or more cell death genes identified as showing a significant difference in expression between normal and NSCLC tissue.

11. The method of predicting whether or not a cancer patient is suitable for chemotherapy according to claim 1, further comprising the step of:
c) detecting a level of expression of the following genes: Serpin B3 (SERPINB3), Hypothetical Protein FLJ23049 (FLJ23049), Hydroxysteroid (17β) dehydrogenase 2 (HSD17B2), Cell Division Cycle homolog 20 (CDC20), Fibronectin type 3 domain containing 3A (FNDC3A), EF-hand calcium binding domain 1 (EFCAB1; previous name FLJ11767), Semaphorin 3D (SEMA3D), Cystatin SN (CST1), Sperm Associated Antigen 6 (SPAG6), Potassium inwardly rectifying channel subfamily J member 16 (KCNJ16), Histone 1H2bg (H1ST1H2BG), Testicular Soluble Adenylyl Cyclase (SAC), Gelsolin (GSN), Cellular Retinoic Binding Protein 1 (CRABP1), Carbonic Anhydrase isoform 12 (CA12), Zinc Finger protein 444 (ZNF444), and v-myb myeloblastosis viral oncogene homolog (MYB), Collagen Type IV alpha 3 chain (COL4A3), Angiotensin II receptor type 2 (AGRT2), Cystatin C (CST3), Survivin (BIRC5), Dual specificity phosphatase 6 (DUSP6), TNF superfamily receptor member 21 (TNFRS21), STAT1 (STAT1), Epithelial Membrane Protein 3 (EMP3), Tissue Inhibitor of Metalloprotease-3 (TIMP3); and Nucleophosmin (NPM1); and
d) predicting whether or not the patient is likely to be a responder or non-responder to a platinum based chemotherapy based on a profile of expression of said genes and therefore suitable or otherwise for platinum based chemotherapy treatment.

12. The method according to according to claim 1 wherein the sample of NSCLC tumour tissue wherein has been obtained a small biopsy taken from the tumour when in situ, or has been obtained from the tumour tissue once removed/resected from the patient, during surgery.

13. The method according to claim 1 wherein detection is carried out using labelling of said protein with an appropriate marker molecule.

14. The method according to claim 1 wherein the marker molecule is a labelled or unlabelled antibody or other binding agent specific for said protein.

15. The method according to claim 1 wherein visualisation of the labelled protein is carried out manually using a microscope, or using automated system such as an optical or laser capture microscope (LCM) with associated software.

16. A DNA array for use in predicting whether or not a cancer patient is suitable for chemotherapy such as platinum based chemotherapy, the array comprising or consisting essentially of Serpin B3 and one or more of the following genes or sequence specific fragments thereof: Hypothetical Protein FLJ23049 (FLJ23049), Hydroxysteroid (17β) dehydrogenase 2 (HSD17B2), Cell Division Cycle homolog 20 (CDC20), Fibronectin type 3 domain containing 3A (FNDC3A), EF-hand calcium binding domain 1 (EFCAB1; previous name FLJ11767), Semaphorin 3D (SEMA3D), Cystatin SN (CST1), Sperm Associated Antigen 6 (SPAG6), Potassium inwardly rectifying channel subfamily J member 16 (KCNJ16), Histone 1H2bg (H1ST1H2BG), Testicular Soluble Adenylyl Cyclase (SAC), Gelsolin (GSN), Cellular Retinoic Binding Protein 1 (CRABP1), Carbonic Anhydrase isoform 12 (CA12), Zinc Finger protein 444 (ZNF444), and v-myb myeloblastosis viral oncogene homolog (MYB), and/or Collagen Type IV alpha 3 chain (COL4A3), Angiotensin II receptor type 2 (AGRT2), Cystatin C (CST3), Survivin (BIRC5), Dual specificity phosphatase 6 (DUSP6), TNF superfamily receptor member 21 (TNFRS21), STAT1 (STAT1), Epithelial Membrane Protein 3 (EMP3), Tissue Inhibitor of Metalloprotease-3 (TIMP3); and Nucleophosmin (NPM1), the array being immobilised on a support.

17. A method of predicting a prognosis of a patient's survival following surgical resection of a non-small cell lung cancer tumour (NSCLC) tumour, comprising the steps of:
a) providing a sample of said surgically resected tumour;
b) detecting Serpin B3 expression in said tumour tissue sample; and
c) determining whether or not said tumour was of the squamous cell carcinoma (SCC) or adenocarcinoma (AC) type and detecting lymph node status if said tumour was of the SCC type, wherein if a significant proportion of tumour cells in said sample are expressing Serpin B3 and the type of tumour is an SCC tumour of the N0 or N1 lymph node status, a good prognosis for the patient is predicted and wherein if a significant proportion of the tumour cells in said sample are expressing Serpin B3 and the type of tumour is an AC tumour or SCC tumour of the N2 or N3 lymph node status, a poor prognosis for the patient is predicted.

18. A method for identifying, evaluating and/or monitoring drug candidates for the treatment of NSCLC, comprising adding a candidate drug to a cell or cell free system which is capable of expressing Serpin B3 and detecting whether or not said drug candidate is able to modulate expression of Serpin B3.

19. The method according to claim 18 for identifying evaluating and/or monitoring drug candidates for the treatment of NSCLC, comprising adding a candidate drug to a cell or cell free system which is capable of expressing one or more of the following genes Hypothetical Protein FLJ23049 (FLJ23049), Hydroxysteroid (17β) dehydrogenase 2 (HSD17B2), Cell Division Cycle homolog 20 (CDC20), Fibronectin type 3 domain containing 3A (FNDC3A), EF-hand calcium binding domain 1 (EFCAB1; previous name FLJ11767), Semaphorin 3D (SEMA3D), Cystatin SN (CST1), Sperm Associated Antigen 6 (SPAG6), Potassium inwardly rectifying channel subfamily J member 16 (KCNJ16), Histone 1H2bg (H1ST1H₂BG), Testicular Soluble Adenylyl Cyclase (SAC), Gelsolin (GSN), Cellular Retinoic Binding Protein 1 (CRABP1), Carbonic Anhydrase isoform 12 (CA12), Zinc Finger protein 444 (ZNF444), and v-myb myeloblastosis viral oncogene homolog (MYB), and/or Collagen Type IV alpha 3 chain (COL4A3), Angiotensin II receptor type 2 (AGRT2), Cystatin C (CST3), Survivin (BIRC5), Dual specificity phosphatase 6 (DUSP6), TNF superfamily receptor member 21 (TNFRS21), STAT1 (STAT1), Epithelial Membrane Protein 3 (EMP3), Tissue Inhibitor of Metalloprotease-3 (TIMP3); and Nucleophosmin (NPM1), and detecting whether or not said drug candidate is able to modulate expression of said one or more genes.

20. The method according to claim 18 wherein the cell is from a cell line grown from a NSCLC sample.

21. The method according to claim 18 wherein the cell is isolated directly from tumour tissue.

22. The method according to claim 18 wherein the drug candidate may modulate lung cancer, lung cancer Serpin B3 gene expression and/or protein expression, modulates lung cancer Serpin B3 gene or protein activity, binds to Serpin B3 in a lung cancer tissue or interferes with the binding of Serpin B3 in lung cancer tissue to its substrates.

23. A cell line derived from a sample of NSCLC which cell line displays increased levels of Serpin B3 expression in comparison to wild-type cells, for use in identifying potential anti-cancer agents.

24. The method of predicting whether or not a cancer patient is suitable for chemotherapy, according to claim 6 further comprising the step of:
a) detecting a level of expression of one or more of the following 10 genes:
Collagen Type IV alpha 3 chain (COL4A3), Angiotensin II receptor type 2 (AGRT2), Cystatin C (CST3), Survivin (BIRC5), Dual specificity phosphatase 6 (DUSP6), TNF superfamily receptor member 21 (TNFRS21), STAT1 (STAT1), Epithelial Membrane Protein 3 (EMP3), Tissue Inhibitor of Metalloprotease-3 (TIMP3) and Nucleophosmin (NPM1); and
d) predicting whether or not the patient is likely to be a responder or non-responder to chemotherapy based on a profile of expression of said genes and therefore suitable or otherwise for platinum based chemotherapy treatment.

25. The method according to claim 24 further comprising the detection of one or more cell death genes identified as showing a significant difference in expression between normal and NSCLC tissue.
